(19) United States
(12) Patent Application Publication
(10) Pub. No.: US 2009/0234453 A1
(43) Pub. Date: Sep. 17, 2009

(54) IMPLANT DEVICES

(75) Inventor: Amiram Steinberg, Avihail (IL)

Correspondence Address: HAYNES AND BOONE, LLP;IP Section, 2323 Victory Avenue, Suite 700, Dallas, TX, 75219, US

(73) Assignee: ACTIVE IMPLANTS CORPORATION, Memphis, TN (US)

(21) Appl. No.: 11/908,777

(22) PCT Filed: Mar. 16, 2006
(86) PCT No.: PCT/IL06/00343

§ 371 Date: Sep. 16, 2008

Related U.S. Application Data
(60) Provisional application No. 60/662,940, filed on Mar. 17, 2005.

(51) Int. Cl. A61F 2/28; A61F 2/08; A61B 17/00

(52) U.S. Cl. 623/16.11; 623/13.14; 606/1

(57) ABSTRACT

An orthopedic implant including an implant element adapted to be attached to at least one bone, the implant element including at least one throughgoing bone growth region adapted to be located in bone growth communication with the at least one bone and configured such that bone growth therein creates a grown bone suture binding the implant element to the at least one bone.

### REFERENCE TO RELATED APPLICATIONS

The present application is related to U.S. Provisional Patent Application Ser. No. 60/662,940 filed Mar. 17, 2005, and entitled IMPLANT DEVICES, the disclosure of which is hereby incorporated by reference and priority of which is hereby claimed pursuant to 37 CFR 1.78(a) (4) and (5)(i).

### FIELD OF THE INVENTION

The present invention relates to orthopedic implants generally and to methodologies and tools useful therewith.

### BACKGROUND OF THE INVENTION

The following patent publications, the disclosures of which are hereby incorporated by reference, are believed to represent the current state of the art:

PCT Patent Application Publication Nos.: WO 97/10776; WO 98/46169; WO 00/053077; WO 2003/047470 and WO 2003/099156

### SUMMARY OF THE INVENTION

The present invention seeks to provide improved orthopedic implants as well as methodologies and tools useful therewith.

There is thus provided in accordance with a preferred embodiment of the present invention an orthopedic implant including an implant element adapted to be attached to at least one bone, the implant element including at least one throughgoing bone growth region adapted to be located in bone growth communication with the at least one bone and configured such that bone growth therein creates a grown bone suture binding the implant element to the at least one bone.

There is also provided in accordance with another preferred embodiment of the present invention an orthopedic implant including a resilient implant element adapted to be attached to at least one bone, the implant element including at least one bone growth region adapted to be located in bone growth communication with the at least one bone and configured such that bone growth therein creates a grown bone lock binding the resilient implant element to the at least one bone.

There is further provided in accordance with yet another preferred embodiment of the present invention an orthopedic implant including an implant element adapted to be attached to at least one bone, the implant element including at least one bone growth region adapted to be located in bone growth communication with the at least one bone and containing grown bone enhancing material configured such that bone growth therein creates a grown bone lock binding the implant element to the at least one bone.

Preferably, the grown bone enhancing material includes bone growth enhancing material. Alternatively, the grown bone enhancing material includes grown bone reinforcing material.

In accordance with a preferred embodiment the implant element is a glenoid socket element. Alternatively, the implant element is an elbow joint implant element. Further alternatively, the implant element is a hip joint implant element. In another alternative embodiment, the implant element is a knee joint implant element. In an additional alternative embodiment, the implant element is a spinal disc implant element.

Preferably, the at least one bone growth region contains a resorbable material. Additionally or alternatively, the at least one bone growth region contains a resorbable grown bone enhancing material. Additionally, the resorbable grown bone enhancing material includes at least one of a bone substitute, a synthetic matrix substitute, a BMP material, a PTH material, a bisphosphonate material, a receptor agonist material and a bone graft substitute.

Preferably, the implant element also includes reinforcement elements located in the at least one bone growth region. Additionally or alternatively, at least part of the at least one bone growth region is formed with an undercut in order to enhance grown bone locking thereat.

Preferably, the at least one bone growth region also includes at least one drug supply channel, the at least one drug supply channel containing a drug which it is sought to supply to at least one location on the at least one bone. Additionally, the drug is in a timed-release dosage form. Additionally or alternatively, the drug is at least one of a taxane, an alkylating agent and an anthracycline.

In another preferred embodiment, the implant is a grown bone locked splint implant. Additionally, the grown bone locked splint implant includes a plurality of throughgoing bone growth regions adapted to be located in bone growth communication with portions of the at least one bone and configured such that bone growth therein creates a grown bone suture binding the grown bone locked splint implant to the portions of the at least one bone. Additionally or alternatively, the grown bone locked splint implant is resilient.

Additionally or alternatively, the grown bone locked splint implant is adapted to be attached to the portions of the at least one bone and configured such that the bone growth creates a grown bone lock binding the grown bone locked splint implant to the at least one bone. Additionally or alternatively, the plurality of bone growth regions include grown bone enhancing material such that the bone growth creates a grown bone lock binding the grown bone locked splint implant to the at least one bone.

In accordance with a preferred embodiment the grown bone locked splint implant is a humerus splint implant and includes a grown bone locked humerus splint implant element arranged to surround portions of the humerus. Alternatively, the grown bone locked splint implant is a femur splint implant and includes a grown bone locked femur splint implant element arranged to surround portions of the femur. Further alternatively, the grown bone locked splint implant is a tibia splint implant and includes a grown bone locked tibia splint implant element arranged to surround portions of the tibia.

Additionally or alternatively, the grown bone locked splint implant is in the form of a split cylinder, formed of a somewhat resilient material, and includes a plurality of elongate reinforcing strips, formed of a generally non-stretchable material, embedded therein. Additionally, the split cylinder has formed therein a plurality of throughgoing bone growth regions which encircle at least one of the reinforcing strips. Additionally or alternatively, the grown bone locked splint implant includes reinforcement elements which encircle at least one of the plurality of reinforcing strips.

Preferably, the grown bone locked splint implant is useful for broken elongated bones as well as for weakened bones.

There is also provided in accordance with another preferred embodiment of the present invention an orthopedic implant including a ligament attachment assembly adapted to be attached to at least one bone and at least one ligament, the ligament attachment assembly including at least one ligament attachment element having at least one throughgoing bone growth region adapted to be located in bone growth communication with the at least one bone and configured such that bone growth therein creates a grown bone suture binding the at least one ligament attachment element to the at least one bone.

There is further provided in accordance with still another preferred embodiment of the present invention an orthopedic implant including a ligament attachment assembly adapted to be attached to at least one bone, the ligament attachment assembly including at least one ligament attachment element having at least one bone growth region adapted to be located in bone growth communication with the at least one bone and configured such that bone growth therein creates a grown bone lock binding the at least one ligament attachment element to the at least one bone.

There is yet further provided in accordance with yet another preferred embodiment of the present invention an orthopedic implant including a ligament attachment assembly adapted to be attached to at least one bone, the ligament attachment assembly including at least one ligament attachment element having at least one bone growth region adapted to be located in bone growth communication with the at least one bone and containing grown bone enhancing material configured such that bone growth therein creates a grown bone lock binding the at least one ligament attachment element to the at least one bone.

Preferably, the grown bone enhancing material includes bone growth enhancing material. Alternatively, the grown bone enhancing material includes grown bone reinforcing material.

Preferably, the at least one bone growth region contains a resorbable material. Additionally, the at least one bone growth region contains a resorbable grown bone enhancing material. Additionally, the resorbable grown bone enhancing material includes at least one of a bone substitute, a synthetic matrix substitute, a BMP material, a PTH material, a bisphosphonate material a receptor agonist material and a bone graft substitute.

Preferably, the orthopedic implant also includes reinforcement elements located in the at least one bone growth region. Additionally or alternatively, at least part of the at least one bone growth region is formed to engage an undercut in bone in order to enhance grown bone locking thereat.

In accordance with a preferred embodiment the ligament attachment assembly is a knee ligament attachment assembly. Alternatively, the ligament attachment assembly is a hip ligament attachment assembly. Further alternatively, the ligament attachment assembly is a shoulder ligament attachment assembly.

Preferably, the ligament attachment assembly includes a plurality of ligament attachment elements, each adapted to have looped therearound, at a ligament loop retaining location thereof, a closed looped ligament end. Additionally, the closed loop ligament ends are formed of at least one of natural ligaments, artificial ligaments and portions thereof. Additionally or alternatively, the plurality of ligament attachment elements are formed of or include bone growth enhancement materials. Additionally or alternatively, the each of the plurality of ligament attachment elements is adapted to be located in bone growth communication with a bone surface and configured such that bone growth therein creates a grown bone ligament anchor.

In accordance with another preferred embodiment, the ligament attachment assembly includes multiple ligament attachment elements and a ligament attachment element positioner. Additionally, the ligament attachment element positioner includes an elongate element formed with a generally conical surface at an inner end thereof. Additionally, the ligament attachment element positioner has first and second operative orientations, the first operative orientation where the generally conical surface is generally decoupled from the multiple ligament attachment elements and allows them to lie at a relatively radially inward position for insertion into a hole formed into bone and the second operative orientation where the generally conical surface engages inner surfaces of the multiple ligament attachment elements and forces the implant elements radially outwardly such that respective first surfaces of the multiple ligament attachment elements engage a first circumferential bone surface of an undercut formed in the at least one bone and respective second surfaces of the multiple ligament attachment elements engage a second circumferential bone surface of the undercut.

In another preferred embodiment, the multiple ligament attachment elements are adapted such that following insertion of the ligament attachment assembly and positioning of the ligament attachment element positioner in the second operative orientation, bone growth may take place while the multiple ligament attachment elements are retained such that the respective first surfaces of the multiple ligament attachment elements engage the first circumferential bone surface of the undercut and the respective second surfaces of the multiple ligament attachment elements engage the second circumferential bone surface of the undercut.

There is also provided in accordance with another preferred embodiment of the present invention an orthopedic implant including a conformal, resilient element adapted to be inserted between respective articulating joint surfaces of a patient, the conformal resilient element including at least one position maintaining engagement portion for attachment to patient tissue in the vicinity of the articulating joint surfaces.

Preferably, the conformal, resilient element is a generally thin element formed of a flexible resilient material and is adapted to be generally surrounded by synovial fluid when implanted. Additionally or alternatively, the conformal, resilient element is formed with at least one throughgoing channel for passage of synovial fluid therethrough in response to changes in fluid pressure resulting from joint articulation and the application of changing forces to a joint. Additionally or alternatively, the conformal, resilient element is operative to decrease or eliminate frictional engagement between the articulating joint surfaces. Alternatively or additionally, the conformal, resilient element is operative to enhance desired cartilage regeneration along at least one of the articulating joint surfaces.

In accordance with a preferred embodiment, the conformal, resilient element is adapted for implantation in a shoulder joint. Alternatively, the conformal, resilient element is adapted for implantation in an elbow joint. Further alternatively, the conformal, resilient element is adapted for implantation in a hip joint. In another alternative embodiment, the conformal, resilient element is adapted for implantation in a knee joint. In an additional alternative embodiment, the conformal, resilient element is adapted for implantation in an ankle joint.

Preferably, the conformal, resilient element is a generally cup-shaped element having an outer facing circumferential protrusion arranged to be loosely seated in a corresponding recess in a surgically reamed socket, with synovial fluid being interposed between the cup-shaped element and the surgically reamed socket and between the cup-shaped element and a corresponding ball. Additionally or alternatively, the conformal, resilient element is formed of polyurethane.

In another alternative embodiment, the conformal resilient element is formed with throughgoing respective non-inclined and inclined passageways to allow synovial fluid to pass therethrough and a relatively thin and highly resilient membrane portion which functions as a synovial fluid pump in response to articulation of the articulating joint surfaces and variations in synovial fluid pressure thereat, thereby enhancing synovial fluid flow through the passageways and reducing friction between the articulating joint surfaces.

Preferably, the conformal resilient element is constructed that immediately following surgical insertion, the shape thereof does not necessarily conform to the corresponding shapes of the articulating joint surfaces and following at least some articulation of the articulating joint surfaces, the shape of the conformal resilient element increasingly conforms to the corresponding shapes of the articulating joint surfaces. Additionally or alternatively, the conformal, resilient element is constructed that during steady-state, long term use, the conformal, resilient element floats in synovial fluid between the articulating joint surfaces. Alternatively, the conformal, resilient element is constructed that during steady-state, long term use, the conformal, resilient element partially floats in synovial fluid between the articulating joint surfaces and partially contacts at least one of the articulating joint surfaces.

Preferably, the conformal, resilient element includes at least one inclined passageway, which provides synovial fluid communication between a first synovial fluid region between the conformal, resilient element and a surgically reamed socket and a second synovial fluid region between the conformal, resilient element and a corresponding ball.

In another preferred embodiment, the conformal, resilient element includes at least one membrane portion defining a synovial fluid pump operative to cause micro-rotation of the conformal, resilient element relative to a socket. Additionally or alternatively, the conformal, resilient element is formed of a liquid absorbing structural material.

Preferably, the conformal, resilient element is also formed with at least one tab having a protrusion adapted to be seated in a corresponding socket surgically formed in a bone. Additionally, engagement of the tab in the socket is snap-fit engagement. Additionally or alternatively, the conformal, resilient element is arranged to be implanted in a knee joint wherein synovial fluid is interposed between the conformal, resilient element and the tibia and between the conformal, resilient element and a corresponding femoral condyle.

Alternatively, the conformal, resilient element is also formed with at least one tab having a resilient protrusion adapted to be seated in a corresponding socket surgically formed in a bone.

Additionally or alternatively, the conformal, resilient element is also formed with at least one tab having an extendible protrusion adapted to be seated in a corresponding socket surgically formed in a bone.

There is further provided in accordance with yet another preferred embodiment of the present invention an orthopedic implant including a resilient, at least partially hollow, meniscus implant element adapted to be inserted between respective articulating knee joint surfaces of a knee joint of a patient, the resilient meniscus implant element including at least one position maintaining engagement portion for attachment to patient tissue in the vicinity of the articulating knee joint surfaces.

Preferably, the meniscus implant element is a generally thin element formed of a flexible resilient material and is adapted to be generally surrounded by synovial fluid when implanted. Additionally or alternatively, the meniscus implant element is formed with at least one throughgoing channel for passage of synovial fluid therethrough in response to changes in fluid pressure resulting from articulation of the knee joint surfaces and the application of changing forces to the knee joint.

Preferably, the meniscus implant element is operative to decrease or eliminate frictional engagement between the articulating knee joint surfaces of the knee joint. Additionally or alternatively, the meniscus implant element is operative to enhance desired cartilage regeneration along at least one of the articulating knee joint surfaces.

Preferably, the meniscus implant element is formed of polyurethane.

In another preferred embodiment, the meniscus implant element is formed with throughgoing passageways to allow synovial fluid to pass therethrough and functions as a synovial fluid pump in response to articulation of the knee joint and variations in synovial fluid pressure thereat, thereby enhancing synovial fluid flow through the passageways and reducing friction in the knee joint.

Preferably, the meniscus implant element is constructed so that immediately following surgical insertion, the shape thereof does not necessarily conform to the corresponding shapes of the articulating joint surfaces and following at least some articulation of the knee joint, the shape of the meniscus implant element increasingly conforms to the corresponding shapes of the articulating joint surfaces. Additionally or alternatively, the meniscus implant element is constructed that during steady-state, long term use, the meniscus implant element floats in synovial fluid between the articulating knee joint surfaces. Alternatively or additionally, the meniscus implant element is constructed that during steady-state, long term use, the meniscus implant element partially floats in synovial fluid between the articulating joint surfaces and partially contacts at least one of the articulating joint surfaces.

Preferably, the meniscus implant element includes at least one inclined passageway, which provides synovial fluid communication between a first synovial fluid region between the meniscus implant element and a surgically reamed socket and a second synovial fluid region between the meniscus implant element and a corresponding ball.

In another preferred embodiment, the meniscus implant element includes at least one membrane portion defining a synovial fluid pump operative to cause micro-rotation of the meniscus implant element relative to a socket.

Preferably, the meniscus implant element is formed of a liquid absorbing structural material.

Additionally or alternatively, the meniscus implant element is also formed with at least one tab having a protrusion adapted to be seated in a corresponding socket surgically formed in a bone. Alternatively or additionally, the meniscus implant element is also formed with at least one tab having a resilient protrusion adapted to be seated in a corresponding socket surgically formed in a bone. Additionally, engagement of the tab in the socket is snap-fit engagement. Additionally or alternatively, the meniscus implant element is arranged to be implanted in a knee joint wherein synovial fluid is interposed between the meniscus implant element and the tibia and between the meniscus implant element and a corresponding femoral condyle.

Preferably, the meniscus implant element is also formed with at least one tab having an extendible protrusion adapted to be seated in a corresponding socket surgically formed in a bone. Additionally or alternatively, the meniscus implant element is configured such that articulation of the knee joint may serve to enhance desired cartilage regeneration along one or both of the knee joint articulating surfaces.

Preferably, the meniscus implant element is conformal. Additionally or alternatively, the meniscus implant element is at least one of a medial meniscus implant and a lateral meniscus implant.

Preferably, the meniscus implant element is formed of a liquid absorbing material. Additionally or alternatively, the meniscus implant element is a generally kidney-shaped, partially hollow generally flat element.

Preferably, the meniscus implant element includes at least one resilient protrusion arranged to be tightly seated in a corresponding socket, surgically formed in the tibia. Additionally or alternatively, the meniscus implant element is formed with at least one tab having a resilient protrusion arranged to be tightly seated in a corresponding socket, surgically formed in the tibia.

In another preferred embodiment, the meniscus implant element includes a hollow portion which tapers down to a relatively thin non-hollow portion along a transition line. Additionally, the orthopedic implant also includes at least one valve communicating with the hollow portion. Additionally, or alternatively, the orthopedic implant also includes a fluid at least partially filling the hollow portion.

Preferably, the orthopedic implant also includes apertures formed in at least one wall defining the hollow portion to enable synovial fluid to readily communicate therethrough and to be pumped by changes in the volume of the interior of the hollow portion resulting from articulation of the knee joint.

Preferably, the meniscus implant element includes folded-over sheet material Additionally or alternatively, the meniscus implant element includes a pair of generally web-type elements which are joined along two seams. Alternatively or additionally, the meniscus implant element includes at least one shape stabilizing internal structural element which cooperates with a polymerizeable material in the interior of a hollow portion thereof to help maintain a desired three-dimensional shape of the implant notwithstanding articulation of the knee joint.

There is still further provided in accordance with a further preferred embodiment of the present invention an orthopedic implant including a shock-absorbing ball assembly including a stem mounting portion and a multi-layer shock-absorbing, ball-defining portion defining a generally ball-shaped articulating surface.

Preferably, the ball assembly is formed with at least one throughgoing channel for passage of synovial fluid therethrough in response to changes in fluid pressure resulting from joint articulation and the application of changing forces to a joint. Additionally or alternatively, the ball assembly is configurable in situ to provide an individualized fit with a socket. Additionally, the ball assembly is configured to enhance desired cartilage regeneration along an articulating surface of a socket.

In accordance with a preferred embodiment the ball assembly is adapted for implantation in a shoulder joint. Alternatively, the ball assembly is adapted for implantation in a hip joint.

Preferably, the ball assembly is a generally ball-shaped, partially hollow multi-layer assembly arranged to be tightly seated onto a top portion of a conventional replacement stem.

In accordance with a preferred embodiment the ball assembly includes a replacement stem socket having an outer facing rim, a generally ball shaped hollow element fixed to the stem socket and defining an articulating surface, a core element, disposed within the hollow element and at least one relatively thin reinforcing layer located between the hollow element and the core element.

Preferably, the core element is formed with at least one synovial fluid void. Additionally, the core element is formed with at least one first synovial fluid communication passageway which communicates with the at least one void and a location adjacent the outer facing rim. Additionally, the core element is formed with at least one second synovial fluid communication passageway which communicates between at least one void through the core element, the at least one reinforcing layer and the hollow element with the articulating surface, thus providing synovial fluid communication with the articulating surface.

Preferably, the orthopedic implant also includes at least one one-way flow valve associated with at least one of the first and second synovial fluid communication passageways, whereby pumping action produced by articulation of the ball assembly relative to a socket causes a relatively large amount of synovial fluid to be provided in an articulation region between the articulating surface of the ball assembly and a corresponding articulating surface of the socket. Additionally, the pumping action is constrained by the at least one one-way flow valve such that synovial fluid flows towards the at least one void only via the at least one first passageway and from the at least one void to the articulation region only via the at least one second passageway.

Preferably, the orthopedic implant also includes at least one selectably inflatable, selectably expandable void. Additionally, the at least one selectably inflatable void is located so as to underlie most of a generally spherical articulating surface defined by the ball assembly.

In another preferred embodiment, the orthopedic implant also includes a spherical radius determining material passageway communicating with the selectably inflatable void to enable a spherical radius determining material to be selectably inserted into the selectably inflatable void in order to enable adaptation of the overall radius of the generally spherical articulating surface in situ so as to provide individualized fit of the ball implant to a patient's socket.

Preferably, the orthopedic implant also includes at least one fluid-filled void.

There is even further provided in accordance with yet another preferred embodiment of the present invention a surgical groove reamer including a central bone anchor element including a generally spherical bone engagement surface and a central shaft extending along a longitudinal axis, the central shaft having a first threaded portion thereon, a rotational driving assembly, mounted for rotation about the central shaft and including a first handle fixedly associated with an elongate hollow shaft, which is sized to rotationally accommodate the central shaft, the hollow shaft terminating in a rotational driving plate, a rotational and axial driving element, coupled for rotation together with the rotational driving plate, the rotational and axial driving element including a second threaded portion, which threadably engages the first threaded portion.

Preferably, the rotational driving plate is formed with a plurality of axially extending portions which extend into corresponding axially extending sockets formed in the rotational and axial driving element. Additionally, the rotational driving plate is formed with a plurality of first slidable knife support channels and the rotational and axial driving element is formed with a plurality of correspondingly positioned second slidable knife support channels, which extend generally at an angle with respect to the plurality of first slidable knife support channels.

In another preferred embodiment the surgical groove reamer also includes a plurality of knives, each slidably seated in a corresponding pair of the first and second slidable knife support channels and mounted on a resilient knife support which permits simultaneous radially outward and rotational displacement of the knives in response to simultaneous axial and rotational movement of the rotational and axial driving element in threaded engagement with the first threaded portion in response to rotation of the first handle.

Preferably, the surgical groove reamer also includes a radially displaceable bone engagement assembly including a plurality of flexible engagement elements, each including a hand engageable portion, lying intermediate first and second retaining portions and a radial bone engaging tooth portion. Additionally, the radially displaceable bone engagement assembly includes plural integrally formed flexible engagement elements which are held together about the hollow shaft at respective first and second retaining portions thereof by a corresponding pair of retaining bands to collectively define a second handle at hand engageable portions thereof.

Preferably, the surgical groove reamer is operative such that an operator grasping the second handle with one hand causes bending of the plurality of flexible engagement elements about the first and second retaining portions, causing the bone engaging tooth portions to be displaced radially outwardly into retaining engagement with walls of a bone socket being reamed.

There is also provided in accordance with still another preferred embodiment of the present invention a socket implanting assembly including a socket implant assembly including inner and outer implantable socket elements and a socket element mounting assembly adapted for preassembly with the inner and outer implantable socket elements and an implanter tool operative with the socket implant assembly for implanting the implantable socket elements in a reamed socket in a patient's joint.

There is further provided in accordance with another preferred embodiment of the present invention a socket implanting assembly including an implanter tool useful with a socket implant assembly including inner and outer implantable socket elements and a socket element mounting assembly adapted for preassembly with the inner and outer implantable socket elements for implanting the implantable socket elements in a reamed socket in a patient's joint.

There is yet further provided in accordance with still another preferred embodiment of the present invention a socket implanting assembly for use with an implanter tool and including a socket implant assembly including inner and outer implantable socket elements and a socket element mounting assembly adapted for preassembly with the inner and outer implantable socket elements.

There is even further provided in accordance with yet another preferred embodiment of the present invention a socket implanting assembly for use with an implanter tool and inner and outer implantable socket elements, the socket implanting assembly including a socket implant mounting assembly adapted for preassembly with the inner and outer implantable socket elements and for implanting operation in cooperation with the implanter tool.

Preferably, the implanter tool includes a central shaft extending along an axis and having an outer facing threaded portion adjacent the top thereof and a rotational driving assembly mounted onto the central shaft for driving rotation thereof, the rotational driving assembly including a handle fixed to the central shaft. Additionally, the central shaft is formed with an end portion having a reduced radius and defining a shoulder with respect to the remainder of the central shaft. Additionally, the end portion has an end surface formed with a bevel.

In another preferred embodiment, the socket implanting assembly also includes a sleeve disposed about the central shaft, the sleeve including a generally uniform inner surface and an inner facing threaded portion. Additionally, the sleeve includes an outer surface which includes a circumferential recess, defining a retaining circumferential protrusion which may be engaged by a protective sleeve, depending from the handle. Further, a central portion of the outer surface of the sleeve is knurled in order to provide a gripping surface for user engagement during implanting.

Preferably, the outer surface includes a peripheral protrusion of uniform radius and, spaced therefrom along the axis, a beveled protrusion, which tapers inwardly and towards a bottom edge of the sleeve, a space between the peripheral protrusion and the beveled protrusion defining a recess. Additionally or alternatively, the socket element mounting assembly is arranged for operational engagement with the central shaft and the sleeve.

Preferably, the socket element mounting assembly includes a flexible central element, a pair of outer elements and an end element. Additionally, the flexible central element is formed of a relatively rigid plastic material and is generally rotationally symmetric. Additionally, the flexible central element is formed with a generally flat top surface which surrounds a generally axial collar and is joined thereto by a beveled edge. Additionally, extending outwardly and downwardly from the top surface is an upper peripheral outer surface and extending downwardly from the upper peripheral outer surface is an intermediate peripheral outer surface which terminates in a peripheral recess and below the peripheral recess is a lower peripheral surface which terminates in a lower edge, there being provided, interiorly of the intermediate peripheral outer surface, the peripheral recess and the lower peripheral surface, an interior facing recess which defines an upper interior facing shoulder.

Preferably, a plurality of radially outward extending tabs are formed at a junction of the upper peripheral outer surface and the intermediate peripheral outer surface, and a plurality of slits are formed in the flexible central element to provide flexibility thereof and extend radially in evenly spaced azimuthal distribution through the flat top surface and the upper peripheral outer surface.

In another preferred embodiment, the socket implanting assembly also includes a bottom element formed with a central collar portion which is integrally joined at its bottom to a curved generally radially extending portion having a peripheral edge which is configured to seat in a recess of the flexible central portion against the upper interior facing shoulder. Additionally, a plurality of radially extending ribs are provided at the top of the radially extending portion and a ring integrally depends from the radially extending portion slightly inwardly of the peripheral edge, the ring defining a peripheral outwardly facing surface and an adjacent downwardly facing surface.

Preferably, the outer elements together define a generally hemispherical body and each include a generally flat top facing surface which defines a collar, an upper peripheral surface, an intermediate peripheral surface and a lower peripheral surface.

Preferably, apertures are formed in the intermediate peripheral surface to accommodate the tabs of the flexible central element for desired mounting of the outer elements with respect thereto. Additionally, the apertures each include a relatively widened portion for receiving one of the tabs and a relatively narrowed portion adjacent the widened portion for retaining the one of the tabs.

Preferably, the inner implantable socket element includes a relatively rigid implant element. Additionally, the inner implantable socket element includes a generally circular peripheral rim below which is defined a generally circular, peripheral, outer-facing recess and a generally spherical outer surface portion, the inner implantable socket element defining at an interior surface thereof, a generally spherical articulating surface. Additionally, the peripheral rim is formed with a beveled edge.

Preferably, the outer implantable socket element includes a relatively resilient implant element. Additionally, the outer implantable socket element includes a generally circular inwardly facing peripheral rim, which is adapted to be seated in the outer-facing recess of the inner implantable socket element, and, disposed below the circular inwardly facing peripheral rim, an inwardly tapered portion which terminates in an inwardly facing peripheral protrusion having a downwardly facing beveled edge adapted to engage the beveled edge of the inner implantable socket element. Additionally, the outer implantable socket element also includes, below the downwardly facing beveled edge, an inwardly facing recess which is adapted to engage the peripheral rim of the inner implantable socket element and below the inwardly facing recess, an inwardly facing protrusion which is adapted to engage the outwardly facing recess of the inner implantable socket element.

Preferably, the inwardly tapered portion of the outer implantable socket element lying below the inwardly facing peripheral protrusion is generally spherical and is adapted to engage the spherical outer surface portion of the inner implantable socket element and wherein an outer surface of the outer implantable socket element is generally spherical and includes a peripheral protrusion which lies intermediate therealong. Additionally, reinforcing material is provided at least one location between the inwardly tapered portion and the outer surface of the outer implantable socket element. Additionally, the reinforcing material is provided generally at a location which lies against a naturally occurring acetabulum notch in a patient's acetabulum.

Preferably, the inner implantable socket element includes a generally smooth outer surface which includes a circumferential protrusion intermediate therealong. Additionally, the outer implantable socket element includes a generally smooth outer surface and is adapted to be forced by the circumferential protrusion into locking engagement with a corresponding reamed recess formed in a patient's acetabulum.

There is also provided in accordance with another preferred embodiment of the present invention a method of alleviating difficulties in joint articulation of a patient including placing a conformal, resilient element between respective articulating joint surfaces of the patient and retaining the conformal resilient element in a desired position with respect to the respective articulating joint surfaces by attachment thereof to patient tissue in the vicinity of the articulating joint surfaces.

There is further provided in accordance with yet another preferred embodiment of the present invention a method of implanting a bone implant element including a bone growth region, the method including the steps of positioning the bone implant element with the bone growth region in contact with a patient's bone surface, encouraging bone growth from the bone surface into the bone growth region and completing bone growth throughout the bone growth region, thereby locking the bone implant element to the bone surface within a few months.

There is still further provided in accordance with still another preferred embodiment of the present invention a method of implanting a bone implant element including at least one bone growth region and at least one drug delivery channel communicating therewith, the method including the steps of positioning the bone implant element with the at least one bone growth region in contact with a patient's bone surface, encouraging bone growth from the bone surface into the at least one bone growth region, effecting drug delivery through the at least one drug delivery channel to the bone surface and completing bone growth throughout the at least one bone growth region, thereby locking the bone implant element to the bone surface.

There is yet further provided in accordance with a further preferred embodiment of the present invention a method of implanting a bone splint implant element including at least two bone growth regions, the method including the steps of positioning the bone splint implant element with at least one of the at least two bone growth regions in contact with bone surfaces of each of at least two bone elements, encouraging bone growth from each of the bone surfaces into the at least two bone growth regions and completing bone growth throughout the at least two bone growth regions, thereby locking the bone splint implant element to the bone surfaces.

There is also provided in accordance with another preferred embodiment of the present invention a method of implanting a ligament attachment assembly including at least one bone growth region, the method including the steps of forming a hole in a bone in a manner providing a circumferential undercut, having a first circumferential surface and therebelow a second circumferential surface which are joined to define the circumferential undercut, providing the ligament attachment assembly, including a plurality of ligament attachment elements, each having looped therearound, at a ligament loop retaining location thereof, a closed looped ligament end and inserting the ligament attachment assembly into the hole and locating the plurality of ligament attachment elements in bone growth communication with a bone surface defined by the hole and configured such that bone growth thereat creates a grown bone ligament anchor binding the plurality of ligament attachment elements to the bone.

Preferably, the ligament attachment assembly includes a ligament attachment element positioner in the form of an elongate element having a generally conical surface at an inner end thereof, and the method also includes the steps of orienting the ligament attachment element positioner in a first operative orientation where the generally conical surface is generally decoupled from the plurality of ligament attachment elements and allows the plurality of ligament attachment elements to lie at a relatively radially inward position for insertion into the hole and thereafter orienting the ligament attachment element positioner in a second operative orientation where the generally conical surface engages inner surfaces of the plurality of ligament attachment elements, forces the plurality of ligament attachment elements radially outwardly such that respective first surfaces of the plurality of ligament attachment elements engage the first circumferential surface of the circumferential undercut and respective second surfaces of the plurality of ligament attachment elements engage the second circumferential surface of the circumferential undercut.

Additionally, the method of implanting a ligament attachment assembly also includes following the inserting and the orienting the ligament attachment element positioner in the second operative orientation, allowing bone growth to take place while the plurality of ligament attachment elements are retained such that the first respective surfaces of the plurality of ligament attachment elements engage the first circumferential surface of the circumferential undercut and the second respective surfaces of the plurality of ligament attachment elements engage the second circumferential surface of the circumferential undercut, thereby providing a grown bone lock binding the plurality of ligament attachment elements to the bone, thus creating the grown bone ligament anchor.

There is also provided in accordance with another preferred embodiment of the present invention a method of implanting an inter-surface articulating joint implant, the method including the steps of surgically inserting the inter-surface articulating joint implant between an acetabulum socket and a femoral head when the inter-surface articulating joint implant does not necessarily conform to corresponding shapes of articulating surfaces of the acetabulum socket and the femoral head, by means of at least some articulation of the hip joint, causing the shape of the inter-surface articulating joint implant to become increasingly conformal to the corresponding shapes of the articulating surfaces of the acetabulum socket and the femoral head and causing the inter-surface articulating joint implant to float in synovial fluid between the articulating surfaces of the acetabulum socket and the femoral head.

There is further provided in accordance with still another preferred embodiment of the present invention a method of implanting an inter-surface articulating joint implant, the method including the steps of surgically inserting the inter-surface articulating joint implant between an acetabulum socket and a femoral head when the inter-surface articulating joint implant does not necessarily conform to corresponding shapes of articulating surfaces of the acetabulum socket and the femoral head, by means of at least some articulation of the hip joint, causing the shape of the inter-surface articulating joint implant to become increasingly conformal to the corresponding shapes of the articulating surfaces of the acetabulum socket and the femoral head and causing the inter-surface articulating joint implant to partially float in synovial fluid between the articulating surfaces of the acetabulum socket and the femoral head and to partially contact at least one of the acetabulum socket and the femoral head.

Preferably, the method of implanting an inter-surface articulating joint implant also includes providing synovial fluid communication between a first synovial fluid region defined between the inter-surface articulating joint implant and the acetabulum socket and a second synovial fluid region defined between the inter-surface articulating joint implant and the femoral head. Additionally, the method of implanting an inter-surface articulating joint implant also includes pumping synovial fluid and thus providing micro-rotation of the inter-surface articulating joint implant relative to the acetabulum socket, thereby to tend to prevent the inter-surface articulating joint from being frozen in position relative to either of the articulating surfaces of the acetabulum socket and the femoral head and to contribute to lowering friction between the inter-surface articulating joint implant and the articulating surfaces of the acetabulum socket and the femoral head.

There is still further provided in accordance with yet another preferred embodiment of the present invention a method of implanting an inter-surface articulating joint implant, the method including the steps of surgically inserting the inter-surface articulating joint implant between a tibia and a femur head when the inter-surface articulating joint implant does not necessarily conform to corresponding shapes of articulating surfaces of the tibia and the femur head, by means of at least some articulation of the knee joint, causing the shape of the inter-surface articulating joint implant to become increasingly conformal to the corresponding shapes of the articulating surfaces of the tibia and the femur head and causing the inter-surface articulating joint implant to float in synovial fluid between the articulating surfaces of the tibia and the femur head.

There is even further provided in accordance with yet another preferred embodiment of the present invention a method of implanting an inter-surface articulating joint implant, the method including the steps of surgically inserting the inter-surface articulating joint implant between a tibia and a femur when the inter-surface articulating joint implant does not necessarily conform to corresponding shapes of articulating surfaces of the tibia and the femur, by means of at least some articulation of the knee joint, causing the shape of the inter-surface articulating joint implant to become increasingly conformal to the corresponding shapes of the articulating surfaces of the tibia and the femur and causing the inter-surface articulating joint implant to partially float in synovial fluid between the articulating surfaces of the tibia and the femur and to partially contact at least one of the tibia and the femur.

Preferably, the method of implanting an inter-surface articulating joint implant also includes providing synovial fluid communication between a first synovial fluid region defined between the inter-surface articulating joint implant and the tibia and a second synovial fluid region defined between the inter-surface articulating joint implant and the femur. Additionally, the method of implanting an inter-surface articulating joint implant also includes pumping synovial fluid, thereby to contribute to lowering friction between the inter-surface articulating joint implant and the articulating surfaces of the tibia and the femur.

There is also provided in accordance with another preferred embodiment of the present invention a method of implanting a meniscus implant, the method including the steps of surgically inserting the meniscus implant between a tibia and a femur when the meniscus implant does not necessarily conform to corresponding shapes of articulating surfaces of the tibia and the femur, by means of at least some articulation of the knee joint, causing the shape of the meniscus implant to become increasingly conformal to the corresponding shapes of the articulating surfaces of the tibia and the femur and causing the meniscus implant to float in synovial fluid between the articulating surfaces of the tibia and the femur.

There is further provided in accordance with yet another preferred embodiment of the present invention a method of implanting a meniscus implant, the method including the steps of surgically inserting the meniscus implant between a tibia and a femur when the meniscus implant does not necessarily conform to corresponding shapes of articulating surfaces of the tibia and the femur, by means of at least some articulation of the knee joint, causing the shape of the meniscus implant to become increasingly conformal to the corresponding shapes of the articulating surfaces of the tibia and the femur and causing the meniscus implant to partially float in synovial fluid between the articulating surfaces of the tibia and the femur and to partially contact at least one of the tibia and the femur.

Preferably, the method of implanting a meniscus implant also includes providing synovial fluid communication between a first synovial fluid region defined between the meniscus implant and the tibia and a second synovial fluid region defined between the meniscus implant and the femur. Additionally, the method of implanting a meniscus implant also includes pumping synovial fluid, thereby to contribute to lowering friction between the meniscus implant and the articulating surfaces of the tibia and the femur.

There is still further provided in accordance with still another preferred embodiment of the present invention a method of employing a shock-absorbing ball implant, the method including the steps of surgically inserting the shock-absorbing ball implant and rigidly mounting it onto a stem, disposing the shock-absorbing ball implant with an articulating surface thereof in articulating engagement with a socket and by walking or running, intermittently applying a force on the shock-absorbing ball implant via the socket, thereby producing deformation of the shock-absorbing ball implant and consequent temporary reshaping of the articulating surface, the deformation producing pumping of synovial fluid into an articulation region between the articulating surface and the socket.

Preferably, the method of employing a shock-absorbing ball implant also includes the step of by walking or running intermittently removing the force, thereby eliminating the deformation of the shock-absorbing ball implant and consequent temporary reshaping of the articulating surface and allowing drainage of the synovial fluid from the articulation region between the articulating surface and the socket.

There is yet further provided in accordance with yet another preferred embodiment of the present invention a method of employing an individually in-situ sizable ball implant, the method including the steps of surgically inserting the individually in-situ sizable ball implant and rigidly mounting it onto a stem, disposing the individually in-situ sizable ball implant with an articulating surface thereof in articulating engagement with a socket and selectably filling at least a portion of the individually in-situ sizable ball implant to cause the articulating surface to have an individually preferred configuration adapted to the socket.

Preferably, the method of employing an individually in-situ sizable ball implant also includes enhancing regeneration of cartilage along the articulating surface by precise matching of the articulating surface and the socket.

There is also provided in accordance with another preferred embodiment of the present invention a method of surgically reaming a bone socket including the steps of providing a reamer including a bone anchor assembly including a generally spherical bone engagement surface and a central shaft extending along a longitudinal axis, the central shaft having a first threaded portion thereon, a rotational driving assembly, mounted for rotation about the central shaft and including a first handle fixedly associated with an elongate hollow shaft, which is sized to rotationally accommodate the central shaft, the hollow shaft terminating in a rotational driving plate, a rotational and axial driving element, coupled for rotation together with the rotational driving plate, the rotational and axial driving element including a second threaded portion, which threadably engages the first threaded portion, the rotational driving plate being formed with a plurality of axially extending portions which extend into corresponding axially extending sockets formed in the rotational and axial driving element and a plurality of first slidable knife support channels and the rotational and axial driving element being formed with a plurality of correspondingly positioned second slidable knife support channels, which extend generally at an angle with respect to the first slidable knife support channels and a plurality of knives, each slidably seated in a pair of corresponding ones of the first and second slidable knife support channels and mounted on a resilient knife support which permits simultaneous radially outward and rotational displacement of the plurality of knives in response to simultaneous axial and rotational movement of the rotational and axial driving element in threaded engagement with the first threaded portion in response to rotation of the first handle, placing the reamer with the bone anchor assembly in a bone socket to be reamed, pushing the first handle axially, thereby causing the generally spherical bone engagement surface to engage a bone surface at the bone socket, thereby anchoring the bone anchor assembly against rotation with respect to the bone socket, producing engagement of radial bone engagement tooth portions of the bone anchor assembly with side surfaces of the bone socket and rotating the first handle about the longitudinal axis, thereby causing rotation of the rotational and axial driving element about the longitudinal axis and consequent corresponding axially forward displacement of the rotational and axial driving element due to threaded engagement of the threaded portion the central shaft with the second threaded portion, the forward displacement driving the plurality of knives which are slidably seated in the first and second slidable knife support channels in a radially outward direction into cutting engagement with the bone socket and the rotation of the first handle about the longitudinal axis causing rotation of the rotational and axial driving element about the longitudinal axis and consequent corresponding axially forward displacement thereof, driving the plurality of knives into cutting engagement with the bone socket, thus producing a circumferential channel therein.

There is further provided in accordance with still another preferred embodiment of the present invention a method for assembly of a socket implant assembly, the method including the steps of mounting a flexible central element on an assembly fixture in a manner preventing relative rotation between the flexible central element and the assembly fixture, mounting a bottom element onto the flexible central element, mounting an inner implant element onto the flexible central element and the bottom element, mounting an outer implant element over the inner implant element and in engagement with the flexible central element and mounting outer elements onto the flexible central element.

There is yet further provided in accordance with yet another preferred embodiment of the present invention a method for mounting a socket implant assembly onto an implanter including the steps of providing the socket implant assembly including a flexible central element, a bottom element, an inner implant element, an outer implant element and outer elements, providing the implanter including a central shaft extending along an axis and having an outer facing threaded portion and an end portion having a reduced radius and defining a shoulder with respect to the remainder of the central shaft, the end portion having an end surface formed with a bevel, a rotational driving assembly mounted onto the central shaft for driving rotation thereof, the rotational driving assembly including a handle fixed to the central shaft and a sleeve disposed about the central shaft, the sleeve including a generally uniform inner surface, an inner facing threaded portion and an outer surface which is conditioned to provide a gripping surface for user engagement during implanting, the outer surface including a peripheral protrusion of uniform radius and spaced therefrom along the axis a beveled protrusion, which tapers inwardly and towards a bottom edge of the sleeve, a space being provided between the peripheral protrusion and the beveled protrusion and defining a recess and providing relative axial displacement of the socket implant assembly and the implanter along the axis, producing snap-fit engagement therebetween.

There is also provided in accordance with another preferred embodiment of the present invention a method for implanting a socket implant, the method including the steps of reaming a bone socket of a patient in order to define a circumferential groove therein, axially inserting a relatively resilient outer implant element into the bone socket and producing snap fit engagement of at least one circumferential protrusion of the relatively resilient outer implant element with the circumferential groove and axially producing engagement of a relatively rigid inner implant element with the outer implant element, thereby to retain the outer implant element in engagement with the bone socket.

Preferably, the method for implanting a socket implant also includes the steps of providing a socket implant assembly including a flexible central element, a bottom element, the inner implant element, the outer implant element and outer elements and employing the socket implant assembly in the axially inserting and the axially producing steps.

Additionally, the method for implanting a socket implant also includes the steps of providing an implanter including a central shaft extending along an axis and having an outer facing threaded portion and an end portion having a reduced radius and defining a shoulder with respect to the remainder of the central shaft, the end portion having an end surface formed with a bevel, a rotational driving assembly mounted onto the central shaft for driving rotation thereof, the rotational driving assembly including a handle fixed to the central shaft and a sleeve disposed about the central shaft, the sleeve including a generally uniform inner surface, an inner facing threaded portion and an outer surface which is conditioned to provide a gripping surface for user engagement during implanting, the outer surface including a peripheral protrusion of uniform radius and spaced therefrom along the axis a beveled protrusion, which tapers inwardly and towards a bottom edge of the sleeve, a space being provided between the peripheral protrusion and the beveled protrusion and defining a recess and employing the implanter for carrying out the axially inserting and the axially producing steps.

Additionally, the method for implanting a socket implant also includes the steps of rotation of the handle about the axis while the sleeve is held static, thus producing axial displacement of the central shaft relative to the sleeve, due to threaded engagement therebetween, and thus relative to the flexible central element, the axial displacement causing displacement of the bottom element and the inner implant element relative to the flexible central element and engagement of an outer surface of the inner implant element with a protrusion of the outer implant element, producing a slight deformation of the at least one circumferential protrusion, preliminary to the engagement between the inner implant element and the outer implant element.

There is also provided in accordance with another preferred embodiment of the present invention a method of implanting an orthopedic implant including attaching an implant element to at least one bone, the implant element including at least one throughgoing bone growth region adapted to be located in bone growth communication with the at least one bone and enhancing bone growth in the bone growth region such that the bone growth therein creates a grown bone suture binding the implant element to the at least one bone.

There is further provided in accordance with yet another preferred embodiment of the present invention a method of implanting an orthopedic implant including attaching a resilient implant element to at least one bone, the resilient implant element including at least one bone growth region adapted to be located in bone growth communication with the at least one bone and allowing bone growth in the at least one bone growth region such that the bone growth therein creates a grown bone lock binding the resilient implant element to the at least one bone.

There is yet further provided in accordance with still another preferred embodiment of the present invention a method of implanting an orthopedic implant including attaching an implant element to at least one bone, the implant element including at least one bone growth region adapted to be located in bone growth communication with the at least one bone and containing grown bone enhancing material and allowing bone growth in the at least one bone growth region such that bone growth therein creates a grown bone lock binding the implant element to the at least one bone.

Preferably, the grown bone enhancing material includes bone growth enhancing material and enhances the bone growth. Alternatively, the grown bone enhancing material includes grown bone reinforcing material which is operative to strengthen the grown bone lock. Additionally or alternatively, the at least one bone growth region contains a resorbable grown bone enhancing material which enhances bone growth.

Preferably, the at least one bone growth region also includes at least one drug supply channel and the method also includes supplying a drug via the at least one drug supply channel to at least one location on the at least one bone.

Preferably, the implant element is a grown bone locked splint implant and includes a plurality of throughgoing bone growth regions adapted to be located in bone growth communication with portions of the at least one bone such that bone growth therein creates a grown bone suture binding the grown bone locked splint implant to the portions of the at least one bone.

There is also provided in accordance with another preferred embodiment of the present invention a method of implanting an orthopedic implant including attaching a ligament attachment assembly to at least one bone and at least one ligament, the ligament attachment assembly including at least one ligament attachment element having at least one throughgoing bone growth region adapted to be located in bone growth communication with the at least one bone and encouraging bone growth in the at least one bone growth region thereby to create a grown bone suture binding the at least one ligament attachment element to the at least one bone.

There is further provided in accordance with still another preferred embodiment of the present invention a method of implanting an orthopedic implant including attaching a ligament attachment assembly to at least one bone, the ligament attachment assembly including at least one ligament attachment element having at least one bone growth region adapted to be located in bone growth communication with the at least one bone and enhancing bone growth in the at least one bone growth region, thereby creating a grown bone lock binding the at least one ligament attachment element to the at least one bone.

There is still further provided in accordance with yet another preferred embodiment of the present invention a method of implanting an orthopedic implant including attaching a ligament attachment assembly to at least one bone, the ligament attachment assembly including at least one ligament attachment element having at least one bone growth region adapted to be located in bone growth communication with the at least one bone and containing grown bone enhancing material and allowing bone growth in the at least one bone growth region to create a grown bone lock binding the at least one ligament attachment element to the at least one bone.

Preferably, the ligament attachment assembly includes multiple ligament attachment elements and a ligament attachment element positioner, which includes an elongate element formed with a generally conical surface at an inner end thereof, and the method also includes initially orienting the ligament attachment element positioner in a first operative orientation where the generally conical surface is generally decoupled from the multiple ligament attachment elements and allows the multiple ligament attachment elements to lie at a relatively radially inward position for insertion into a hole formed in the at least one bone and thereafter orienting the ligament attachment element positioner in a second operative orientation where the generally conical surface engages inner surfaces of the multiple ligament attachment elements and forces the multiple ligament attachment elements radially outwardly such that respective first surfaces of the multiple ligament attachment elements engage a first circumferential bone surface of an undercut formed in the at least one bone and respective second surfaces of the multiple ligament attachment elements engage a second circumferential bone surface of the undercut.

Additionally, following insertion of the ligament attachment assembly and the orienting the ligament attachment element positioner in the second operative orientation, bone growth is allowed to take place while the multiple ligament attachment elements are retained such that the respective first surfaces of the multiple ligament attachment elements engage the first circumferential bone surface of the undercut and the respective second surfaces of the multiple ligament attachment elements engage the second circumferential bone surface of the undercut.

There is also provided in accordance with another preferred embodiment of the present invention a method of implanting an orthopedic implant including inserting a conformal, resilient element between respective articulating joint surfaces of a patient, the conformal resilient element including at least one position maintaining engagement portion and attaching the at least one position maintaining engagement portion to patient tissue in the vicinity of the articulating joint surfaces.

Preferably, the conformal, resilient element is formed with at least one throughgoing channel and synovial fluid is pumped therethrough by changes in fluid pressure resulting from joint articulation and the application of changing forces to a joint. Additionally or alternatively, the conformal, resilient element is operative to enhance desired cartilage regeneration along at least one of the articulating joint surfaces. Alternatively or additionally, the conformal, resilient element is a generally cup-shaped element having an outer facing circumferential protrusion and is loosely seated in a corresponding recess in a surgically reamed socket, with synovial fluid being interposed between the cup-shaped element and the surgically reamed socket and between the cup-shaped element and a corresponding ball.

Preferably, the conformal resilient element is formed with throughgoing respective non-inclined and inclined passageways to allow synovial fluid to pass therethrough and with a relatively thin and highly resilient membrane portion and wherein the membrane functions as a synovial fluid pump in response to articulation of the articulating joint surfaces and variations in synovial fluid pressure thereat, thereby enhancing synovial fluid flow through the passageways and reducing friction between the articulating joint surfaces.

Preferably, immediately following surgical insertion thereof, the shape of the conformal resilient element does not necessarily conform to corresponding shapes of the articulating joint surfaces and following at least some articulation of the articulating joint surfaces, the shape of the conformal resilient element increasingly conforms to the corresponding shapes of the articulating joint surfaces. Additionally or alternatively, conformal, resilient element is operative during steady-state, long term use to float in synovial fluid between the articulating joint surfaces. Alternatively or additionally, the conformal, resilient element is operative during steady-state, long term use to partially float in synovial fluid between the articulating joint surfaces and to partially contact at least one of the articulating joint surfaces.

Preferably, the conformal, resilient element includes at least one inclined passageway, which provides synovial fluid communication between a first synovial fluid region defined between the conformal, resilient element and a surgically reamed socket and a second synovial fluid region defined between the conformal, resilient element and a corresponding ball. Additionally or alternatively, the conformal, resilient element includes at least one membrane portion defining a synovial fluid pump operative to cause micro-rotation of the conformal, resilient element relative to a socket.

There is also provided in accordance with another preferred embodiment of the present invention a method of implanting an orthopedic implant including inserting a resilient, at least partially hollow, meniscus implant element between respective articulating knee joint surfaces of a knee joint of a patient, the resilient meniscus implant element including at least one position maintaining engagement portion and attaching the at least one position maintaining engagement portion to patient tissue in the vicinity of the articulating knee joint surfaces.

Preferably, the meniscus implant element is formed with at least one throughgoing channel and synovial fluid is pumped therethrough by changes in fluid pressure resulting from knee joint articulation and the application of changing forces to a knee joint. Additionally or alternatively, the meniscus implant element is operative to enhance desired cartilage regeneration along at least one of the articulating knee joint surfaces.

Preferably, the meniscus implant element is a generally cup-shaped element having an outer facing circumferential protrusion and is loosely seated in a corresponding recess in a surgically reamed socket, with synovial fluid being interposed between the cup-shaped element and the surgically reamed socket and between the cup-shaped element and a corresponding ball. Additionally or alternatively, the meniscus implant element is formed with throughgoing respective non-inclined and inclined passageways to allow synovial fluid to pass therethrough and a relatively thin and highly resilient membrane portion and wherein the membrane functions as a synovial fluid pump in response to articulation of the articulating knee joint surfaces and variations in synovial fluid pressure thereat, thereby enhancing synovial fluid flow through the passageways and reducing friction between the articulating knee joint surfaces.

Preferably, immediately following surgical insertion thereof, the shape of the meniscus implant element does not necessarily conform to corresponding shapes of the articulating knee joint surfaces and following at least some articulation of the articulating knee joint surfaces, the shape of the meniscus implant element increasingly conforms to the corresponding shapes of the articulating knee joint surfaces. Additionally or alternatively, the meniscus implant element is operative during steady-state, long term use to float in synovial fluid between the articulating knee joint surfaces. Alternatively or additionally, the meniscus implant element is operative during steady-state, long term use to partially float in synovial fluid between the articulating knee joint surfaces and to partially contact at least one of the articulating knee joint surfaces. Alternatively or additionally, the meniscus implant element includes at least one inclined passageway, which provides synovial fluid communication between a first synovial fluid region defined between the meniscus implant element and a surgically reamed socket and a second synovial fluid region defined between the meniscus implant element and a corresponding ball.

There is also provided in accordance with another preferred embodiment of the present invention a method of implanting an orthopedic implant including mounting onto a stem, a shock-absorbing ball assembly including a stem mounting portion and a multi-layer shock-absorbing, ball-defining portion defining a generally ball-shaped articulating surface.

Preferably, the shock-absorbing ball assembly is formed with at least one throughgoing channel and synovial fluid passes therethrough in response to changes in fluid pressure resulting from joint articulation and the application of changing forces to a joint. Additionally or alternatively, the shock-absorbing ball assembly is configurable in situ to provide an individualized fit with a socket. Alternatively or additionally, the shock-absorbing ball assembly enhances desired cartilage regeneration along an articulating surface of a socket.

Preferably, the shock-absorbing ball assembly includes a replacement socket having an outer facing rim, a generally ball shaped hollow element fixed to the stem and defining an articulating surface, at least one relatively thin reinforcing layer; a core element, disposed within the hollow element and separated therefrom by the at least one relatively thin reinforcing layer, the core element being formed with at least one synovial fluid void and at least one first synovial fluid communication passageway which communicates with the at least one synovial fluid void and a location adjacent the outer facing rim and formed with at least one second synovial fluid communication passageway which communicates between the at least one synovial fluid void through the core element, the at least one reinforcing layer and the hollow element with the articulating surface, thus providing synovial fluid communication with the articulating surface and at least one one-way flow valve associated with at least one of the first and second synovial fluid communication passageways; and the method also includes pumping, produced by articulation of the shock-absorbing ball assembly relative to the socket, which causes a relatively large amount of synovial fluid to be provided in an articulation region between the articulating surface of the shock-absorbing ball assembly and a corresponding articulating surface of the socket.

Additionally, the pumping is constrained by the at least one one-way flow valve such that synovial fluid flows towards the at least one synovial fluid void only via the at least one first synovial fluid communication passageway and from the at least one synovial fluid void to the articulation region only via the at least one second synovial fluid communication passageway.

Preferably, the method of implanting an orthopedic implant also includes providing at least one selectably inflatable, selectably expandable void located so as to underlie most of a generally spherical articulating surface defined by the shock-absorbing ball assembly and a spherical radius determining material passageway communicating with the selectably inflatable void and selectably inserting a spherical radius determining material into the selectably inflatable void in order to enable adaptation of the overall radius of the generally spherical articulating surface in situ so as to provide individualized fit of the shock-absorbing ball assembly to a patient's socket.

### BRIEF DESCRIPTION OF THE DRAWINGS

The present invention will be understood and appreciated more fully from the following detailed description, taken in conjunction with the drawings in which:

FIG. 1 is a simplified anatomical illustration showing various applications of a grown bone locked implant constructed and operative in accordance with a preferred embodiment of the present invention;

FIGS. 2A & 2B are simplified partial illustrations of a plurality of alternative structures of a grown bone lockable implant useful in the embodiment of FIG. 1;

FIGS. 3A & 3B are simplified illustrations of sequential bone growth in the embodiments illustrated respectively in FIGS. 2A & 2B;

FIG. 4 is a simplified partial illustration of an alternative embodiment of another grown bone lockable implant useful in the embodiment of FIG. 1;

FIG. 5 is a simplified illustration of sequential bone growth and medicament dispersal in the embodiment illustrated FIG. 4;

FIG. 6 is a simplified anatomical illustration showing various applications of a grown bone locked splint implant constructed and operative in accordance with a preferred embodiment of the present invention;

FIGS. 7A & 7B are simplified partial illustrations of a plurality of alternative structures of a grown bone lockable implant useful in the embodiment of FIG. 6;

FIGS. 8A & 8B are simplified illustrations of sequential bone growth in the embodiments illustrated respectively in FIGS. 7A & 7B;

FIG. 9 is a simplified anatomical illustration showing various applications of a grown bone locked ligament attachment implant constructed and operative in accordance with a preferred embodiment of the present invention;

FIG. 10 is a simplified illustration of steps in the insertion of the ligament attachment implant of FIG. 9 into a bone;

FIGS. 11A & 11B are simplified partial illustrations of a plurality of alternative structures of a grown bone lockable implant useful in the embodiment of FIG. 9;

FIGS. 12A & 12B are simplified illustrations of sequential bone growth in the embodiments illustrated respectively in FIGS. 11A & 11B;

FIG. 13 is a simplified anatomical illustration showing various applications of an inter-surface articulating joint implant constructed and operative in accordance with a preferred embodiment of the present invention;

FIGS. 14A & 14B are simplified partial illustrations of a plurality of alternative structures of an inter-surface articulating joint implant useful in the embodiment of FIG. 13;

FIGS. 15A & 15B are simplified illustrations of an aspect of the functionality of the inter-surface articulating joint implant in the embodiments illustrated respectively in FIGS. 14A & 14B;

FIGS. 16A & 16B are simplified partial illustrations of a plurality of alternative structures of another inter-surface articulating joint implant useful in the embodiment of FIG. 13;

FIGS. 17A & 17B are simplified illustrations of an aspect of the functionality of the inter-surface articulating joint implants in the embodiments illustrated respectively in FIGS. 16A & 16B;

FIG. 18 is a simplified anatomical illustration showing various applications of a meniscus implant constructed and operative in accordance with a preferred embodiment of the present invention;

FIGS. 19A & 19B are simplified partial illustrations of a plurality of alternative structures of a meniscus implant useful in the embodiment of FIG. 18;

FIGS. 20A & 20B are simplified illustrations of an aspect of the functionality of the meniscus implant in the embodiments illustrated respectively in FIGS. 19A & 19B;

FIGS. 21A, 21B, 21C and 21D are sectional illustrations of a plurality of alternative constructions of the meniscus implant of FIGS. 18-20B, taken along the lines XXI-XXI in FIG. 18;

FIG. 22 is a simplified anatomical illustration showing various applications of a ball implant constructed and operative in accordance with a preferred embodiment of the present invention;

FIGS. 23A, 23B and 23C are simplified partial illustrations of a plurality of alternative structures of a ball implant useful in the embodiment of FIG. 22;

FIGS. 24A & 24B are simplified illustrations of aspects of the functionality of the ball implants in the embodiments illustrated respectively in FIGS. 22A & 22B;

FIG. 25 is a simplified pictorial illustration of a groove reamer constructed and operative in accordance with a preferred embodiment of the present invention, useful, for example in the embodiment of FIG. 13;

FIG. 26 is a simplified composite illustration showing the structure of the groove reamer of FIG. 25;

FIGS. 27A, 27B, 27C and 27D are simplified sectional illustrations illustrating various stages of the operation of the groove reamer of FIGS. 25 and 26;

FIG. 28 is a simplified composite exploded-view illustration of a socket implant assembly and implanter useful therewith, constructed and operative in accordance with a preferred embodiment of the present invention;

FIG. 29 is a simplified sectional assembled-view illustration of the socket implant assembly and implanter of FIG. 28;

FIGS. 30A, 30B, 30C, 30D, 30E and 30F are simplified illustrations of assembly of the socket implant assembly of FIGS. 28 and 29;

FIGS. 31A, 31B and 31C are simplified illustrations of mounting of a socket implant assembly onto an assembler;

FIGS. 32A, 32B, 32C and 32D are simplified illustrations of implanting a socket implant employing the apparatus of FIGS. 28 and 29;

FIG. 33 is a simplified composite exploded-view illustration of a socket implant assembly and implanter useful therewith, constructed and operative in accordance with another preferred embodiment of the present invention; and

FIG. 34 is a simplified illustration of a final stage of implanting the socket implant assembly of FIG. 33 into a patient.

### DETAILED DESCRIPTION OF PREFERRED EMBODIMENTS

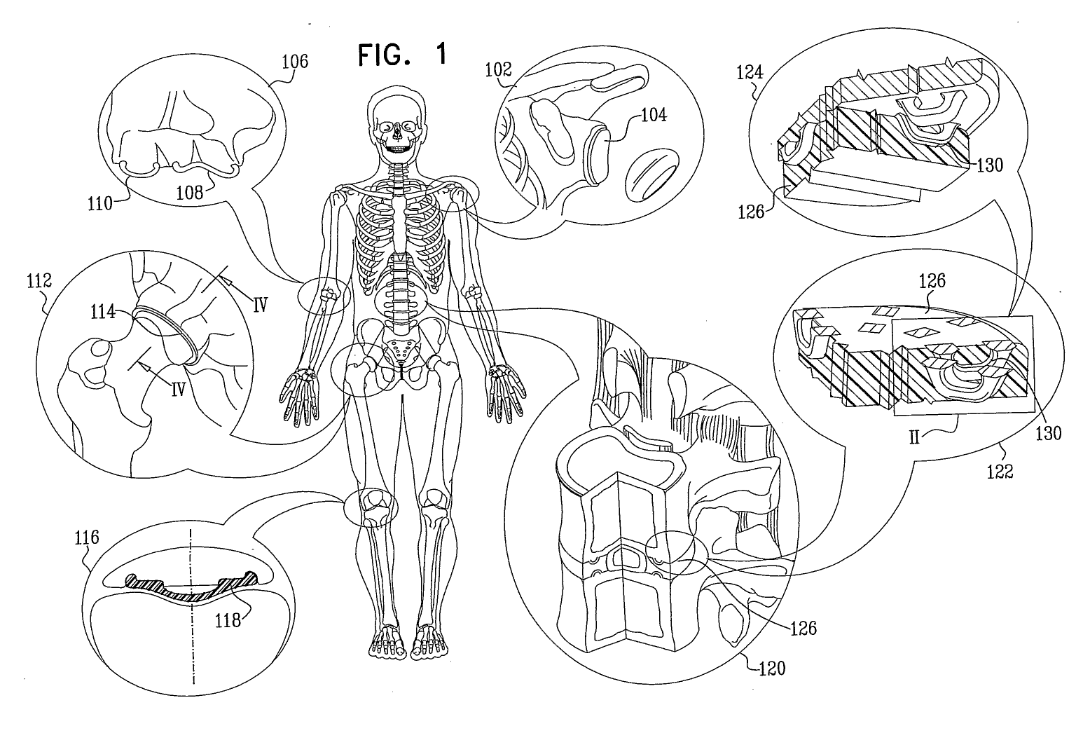

Reference is now made to FIGS. 1-3B which illustrate various applications of a grown bone locked implant constructed and operative in accordance with a preferred embodiment of the present invention.

There is provided in accordance with a preferred embodiment of the present invention an orthopedic implant including an implant element adapted to be attached to at least one bone, the implant element including at least one throughgoing bone growth region adapted to be located in bone growth communication with the bone and configured such that bone growth therein creates a grown bone suture binding the implant element to the bone.

Additionally or alternatively, the implant element may be resilient and be adapted to be attached to at least one bone and may include at least one bone growth region adapted to be located in bone growth communication with the bone and configured such that bone growth therein creates a grown bone lock binding the resilient implant element to the bone.

Additionally or alternatively, the implant element may be adapted to be attached to at least one bone, the implant element including at least one bone growth region adapted to be located in bone growth communication with the at least one bone and containing grown bone enhancing material configured such that bone growth therein creates a grown bone lock binding the implant element to the bone.

The grown bone enhancing material preferably comprises bone growth enhancing material and/or grown bone reinforcing material.

Turning initially to FIG. 1, there is provided a simplified anatomical illustration showing various applications of a grown bone locked implant of the type described hereinabove, constructed and operative in accordance with a preferred embodiment of the present invention. A shoulder joint implant assembly, shown in an enlargement designated by reference numeral 102, includes a grown bone locked artificial glenoid socket element 104 which is constructed and operative in accordance with a preferred embodiment of the present invention.

An elbow joint implant assembly, shown in an enlargement designated by reference numeral 106, includes grown bone locked implant elements 108 and 110, which are constructed and operative in accordance with a preferred embodiment of the present invention.

A hip joint implant assembly, shown in an enlargement designated by reference numeral 112, includes a grown bone locked implant element 114 which is constructed and operative in accordance with a preferred embodiment of the present invention.

A knee joint implant assembly, shown in an enlargement designated by reference numeral 116, includes a grown bone locked implant element 118 which is constructed and operative in accordance with a preferred embodiment of the present invention.

A disc replacement implant assembly, shown in successive enlargements designated by reference numerals 120, 122 and 124, includes a grown bone locked implant element 126 which is constructed and operative in accordance with a preferred embodiment of the present invention. Preferably, the grown bone locked implant element 126 includes a bone growth region 130 which is preferably throughgoing and which is preferably in the shape of a hook or a handle, as shown.

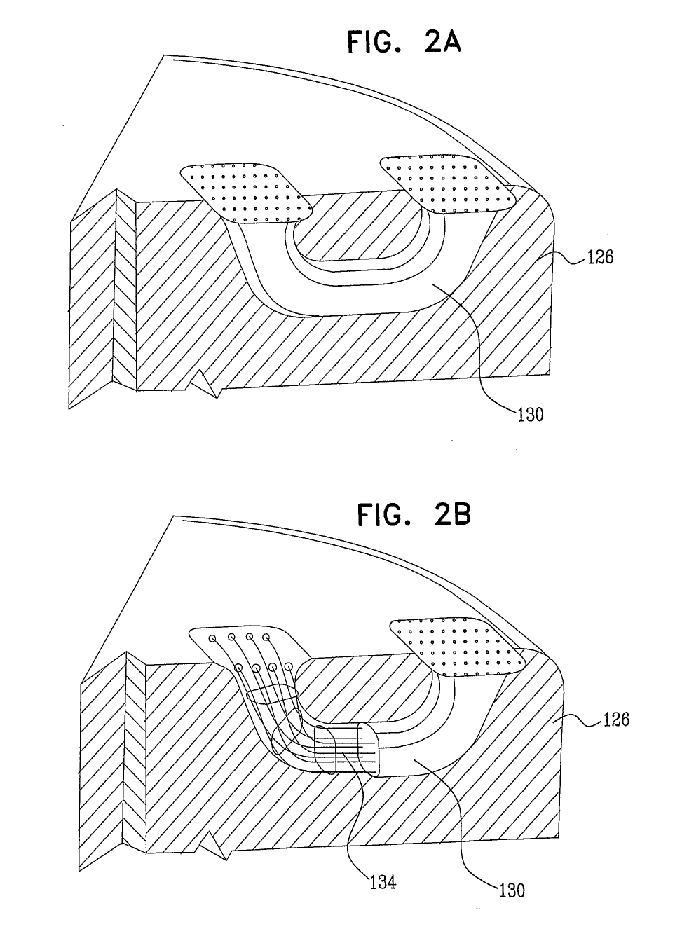

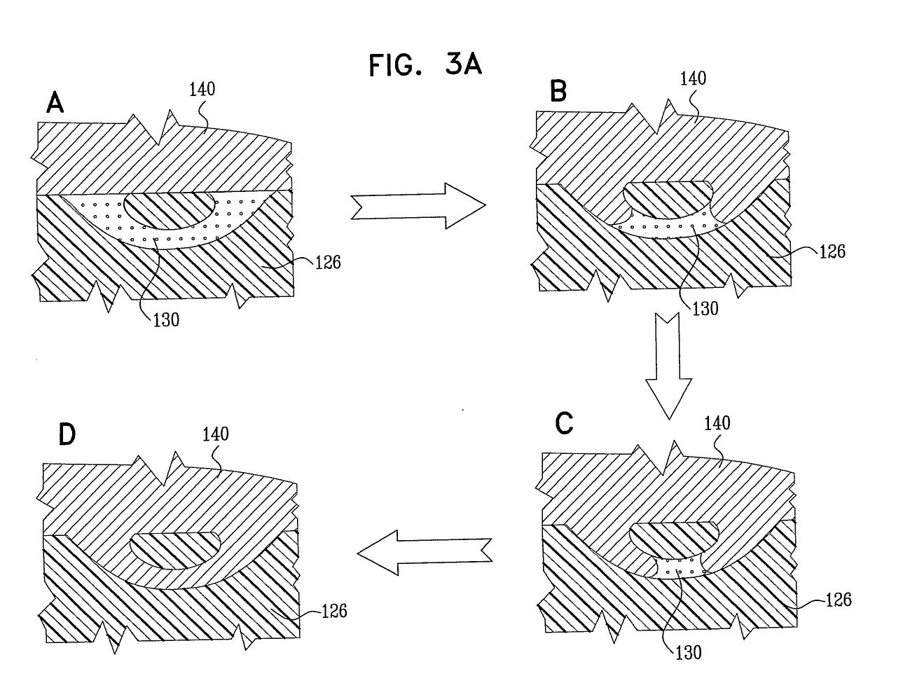

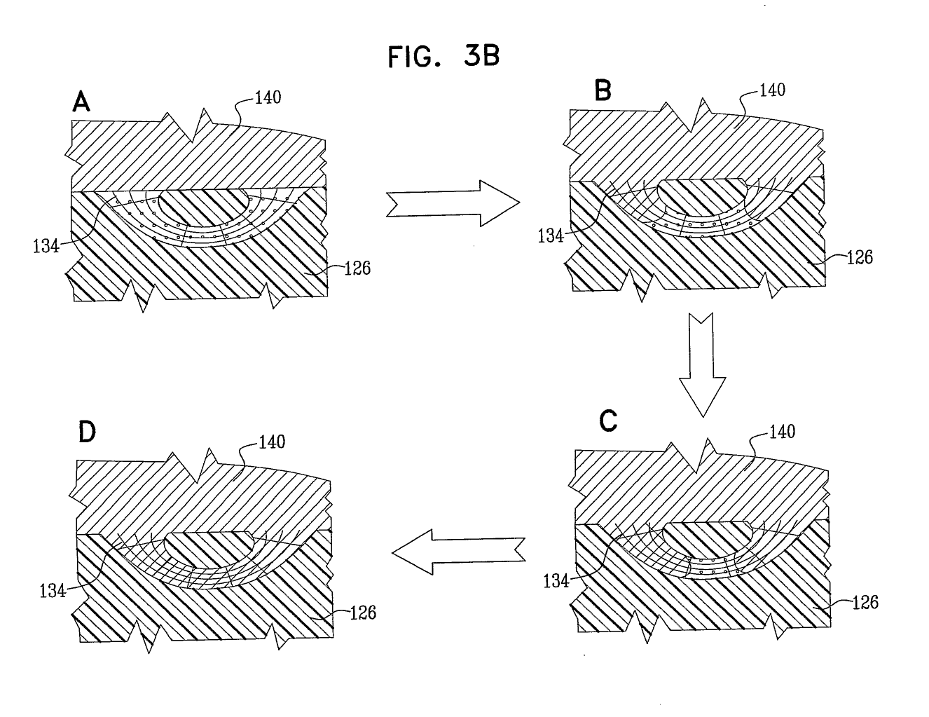

Specific reference is now made to FIGS. 2A & 2B, which are simplified partial illustrations of a plurality of alternative structures of grown bone lockable implant useful in the embodiment of FIG. 1 and to FIGS. 3A & 3B, which are simplified illustrations of sequential bone growth in the embodiments illustrated respectively in FIGS. 2A & 2B.

FIGS. 2A & 2B represent two alternative embodiments of an exemplary implant element useful in a disc replacement implant assembly as shown in enlargement 124 of FIG. 1. FIGS. 2A & 2B are pictorial illustrations of implant element 126 taken generally along lines II-II in enlargement 122. FIGS. 3A & 3B are sectional illustrations of implant element 126 taken generally along the lines II-II. It is appreciated that the descriptions of FIGS. 2A-3B which follow are applicable to any other suitable type of grown bone lockable implant.

Turning now to FIGS. 2A & 2B, there are seen partial illustrations of a portion of a grown bone lockable implant element, such as a replacement spinal disc. FIG. 2A shows a portion of the implant element 126 having formed therein bone growth region 130, which is preferably throughgoing and preferably is in the shape of a hook or a handle. In accordance with a preferred embodiment of the present invention, bone growth region 130 is generally filled with a resorbable material such as any one of the following materials:

α-BSM, commercially available from Etex Corporation of 38 Sidney Street, Cambridge, Mass. 02139;

Bi-Ostetic, commercially available from Berkeley Advanced Biomaterials, Inc., of 901 Grayson St., Suite 101, Berkeley Calif. 94710; and

Boneplast™ Bone Void Filler, commercially available from Interpore Cross International, Inc. of 181 Technology Drive, Irvine, Calif. 92618.

Additionally or alternatively, bone growth region 130 is generally filled with a material, such as any one of the following materials:

a bone graft substitute such as Chronos Granules, commercially available from Synthes, Inc. of 1302 Wrights Lane East, West Chester, Pa. 19380 or POLYGRAFT BGS Technology Bone Graft Substitute, commercially available from OsteoBiologics, Inc. of 12500 Network, Suite 112, San Antonio, Tex. 78249;

a BMP material such as Infuse® Bone Graft, commercially available from Medtronic of 710 Medtronic Parkway Minneapolis, Minn. 55432-5604;

a parathyroid hormone such as Forteo, commercially available from Eli Lilly And Company of Lilly Corporate Center Indianapolis, Ind. 46285 USA; and

a Bisphosphonate material such as Fosamax (alendronate) Bone-strengthening drugs, commercially available from Merck & Co., Inc. of One Merck Drive P.O. Box 100 Whitehouse Station, N.J. 08889-0100 USA.

FIG. 2B shows a portion of the implant element 126 having formed therein a bone growth region 130, which is preferably throughgoing and is preferably in the shape of a hook or a handle. In accordance with a preferred embodiment of the present invention, bone growth region 130 is generally filed with a resorbable material of the type described hereinabove with reference to FIG. 2A. In addition, the implant element of FIG. 2B includes reinforcement elements 134, preferably relatively rigid elongate metal elements or relatively flexible fibers, located in the bone growth region.

Turning now to FIGS. 3A & 3B, there are seen simplified illustrations of sequential bone growth in the embodiments illustrated in FIGS. 2A & 2B respectively. FIGS. 3A and 3B each show at step A the implant element 126 positioned in contact with a vertebral endplate 140. The bone growth region 130 is shown filled with one or more of the materials described hereinabove with reference to FIGS. 2A and 2B. As seen in step A, bone growth has not yet commenced. At steps B and C respectively, initial and further bone growth from endplate 140 into bone growth region 130 are shown. Step D shows complete bone growth throughout the throughgoing bone growth region 130. The entire bone growth process can be expected to take place over a period of a few months.

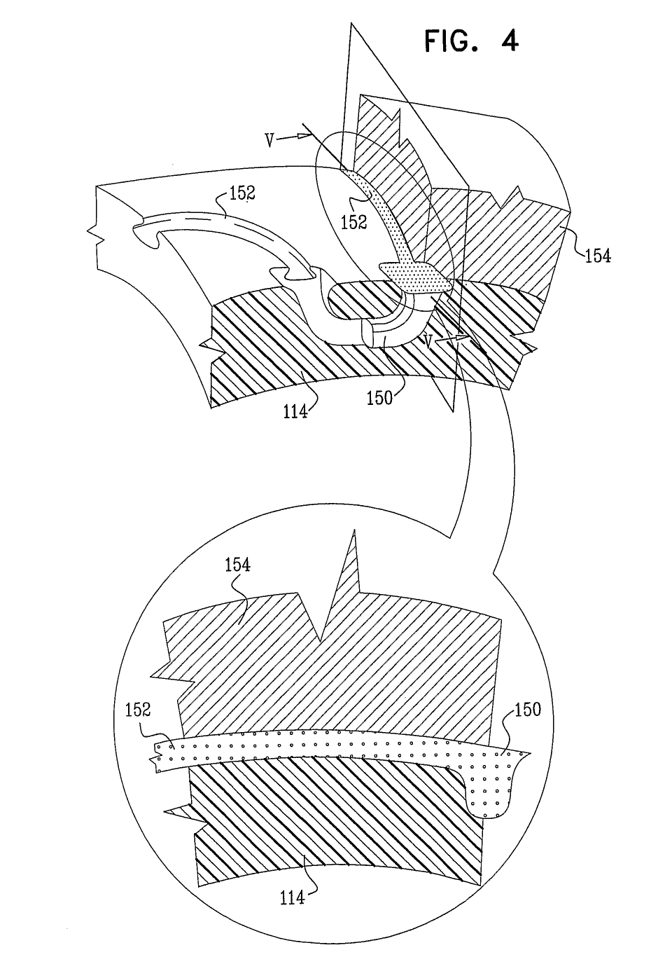

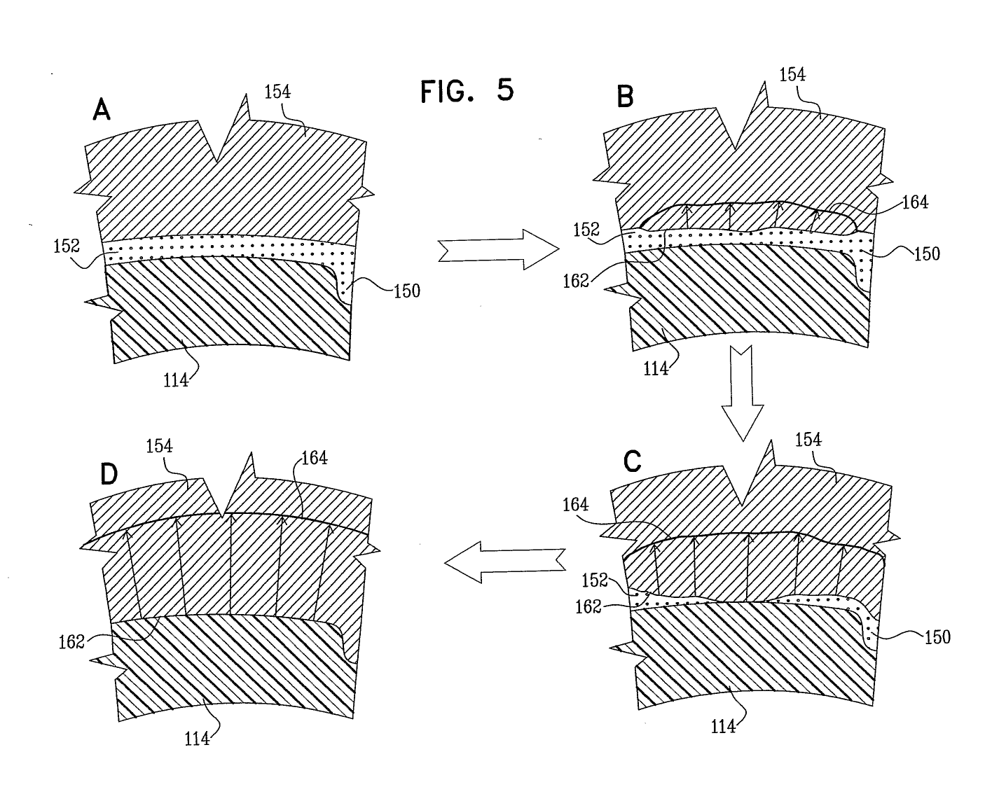

Reference is now made to FIG. 4, which is a simplified partial illustration of a grown bone lockable implant useful in the embodiment of FIG. 1 and to FIG. 5, which is a simplified illustration of sequential bone growth and drug delivery in the embodiment illustrated FIG. 4.

FIG. 4 represents an exemplary implant element useful in a hip replacement implant assembly as shown in enlargement 112 of FIG. 1. FIG. 4 is a pictorial illustration of implant element 114 taken generally along lines IV-IV in enlargement 112. FIG. 5 is a sectional illustration of implant element 114 taken generally along lines V-V in FIG. 4. It is appreciated that the descriptions of FIGS. 4 and 5 which follow are applicable to any other suitable type of grown bone lockable implant.

Turning now to FIG. 4, there is seen a partial illustration of a portion of a grown bone lockable implant element, such as a replacement acetabulum. FIG. 4 shows a portion of the implant element 114 having formed therein a bone growth region 150, which is preferably throughgoing and is preferably in the shape of a hook or a handle. Forming part of or communicating with bone growth region 150 there are preferably provided one or more drug supply channels 152, each preferably containing a drug, preferably in a timed-release dosage form, which it is sought to supply to one or more locations on an adjacent bone 154. Some examples of timed release drugs include:

Taxanes such as Nolvadex (Tamoxifen Citrate) commercially available from Astrazeneca Pharmaceuticals Lp (U.S. Headquarters) of Wilmington, Mass.;

Alkylating agents such as Cytoxan and Neosar which are commercially available from Mead Johnson Oncology Products of Princeton, N.J.;

Anthracyclines such as Adriamycin and Ellence which are commercially available from Pharmacia & Upjohn of 100 Rte. 206 North, Peapack, NJ 07977; and

Platinum compounds such as PLATINOL-AQ, commercially available from Bristol Myers-Squibb Company of Princeton, N.J. 08543.

In accordance with a preferred embodiment of the present invention, channels 152 and bone growth region 150 are generally filled with bone growth enhancing materials of the type described hereinabove with respect to FIGS. 2A & 2B. Channels 152 and other parts of the bone growth region 150 may be formed with an undercut, as shown, in order to enhance grown bone locking thereat. In accordance with an alternative embodiment of the present invention, the implant element containing drug supply channels 152 need not necessarily be a grown bone lockable implant.

Turning now to FIG. 5, there is seen a simplified illustration of sequential bone growth and drug delivery in the embodiment illustrated in FIG. 4. FIG. 5 shows at step A, the implant element 114 positioned in contact with an acetabulum bone 154. The bone growth region 150 and channels 152 are shown filled with one or more of the materials described hereinabove with reference to FIGS. 2A and 2B. Channels 152 also include one or more drugs. As seen at step A, bone growth and drug delivery have not yet commenced. At steps B and C respectively, initial and further bone growth from acetabulum bone 154 into bone growth region 150 are shown at reference numeral 162 and drug delivery through channels 152 to bone 154 is shown at reference numeral 164. Step D shows complete bone growth throughout the throughgoing bone growth region 150 and continuing drug delivery through channels 152. The entire bone growth process can be expected to take place over a period of a few months and the drug delivery can take place over a shorter or longer period.

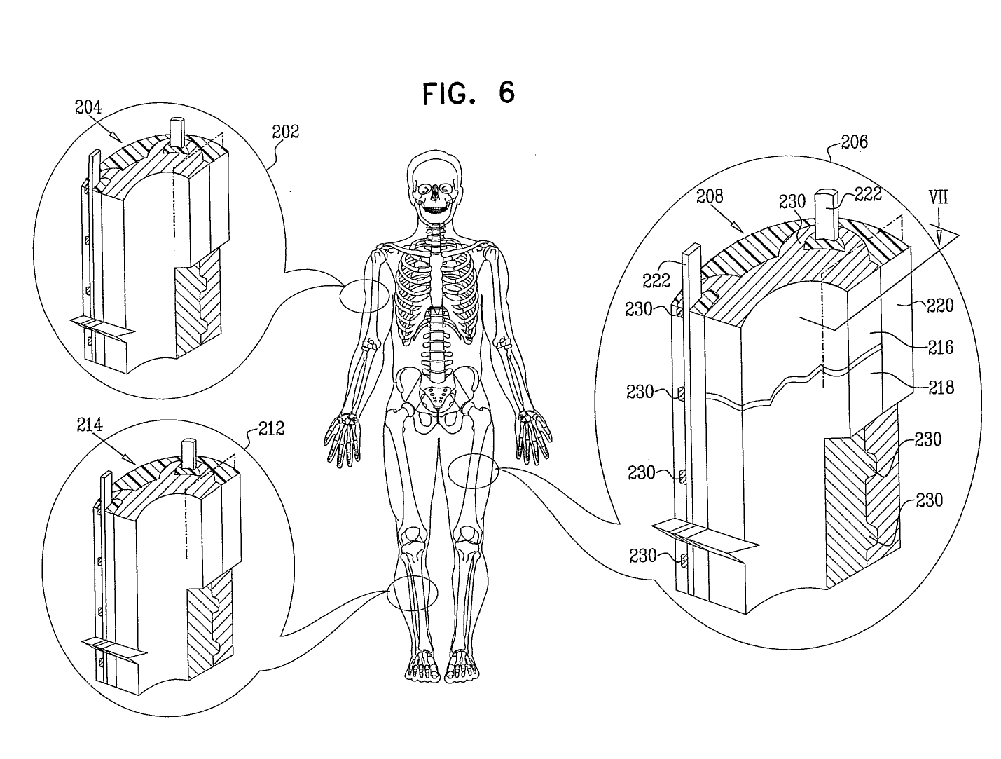

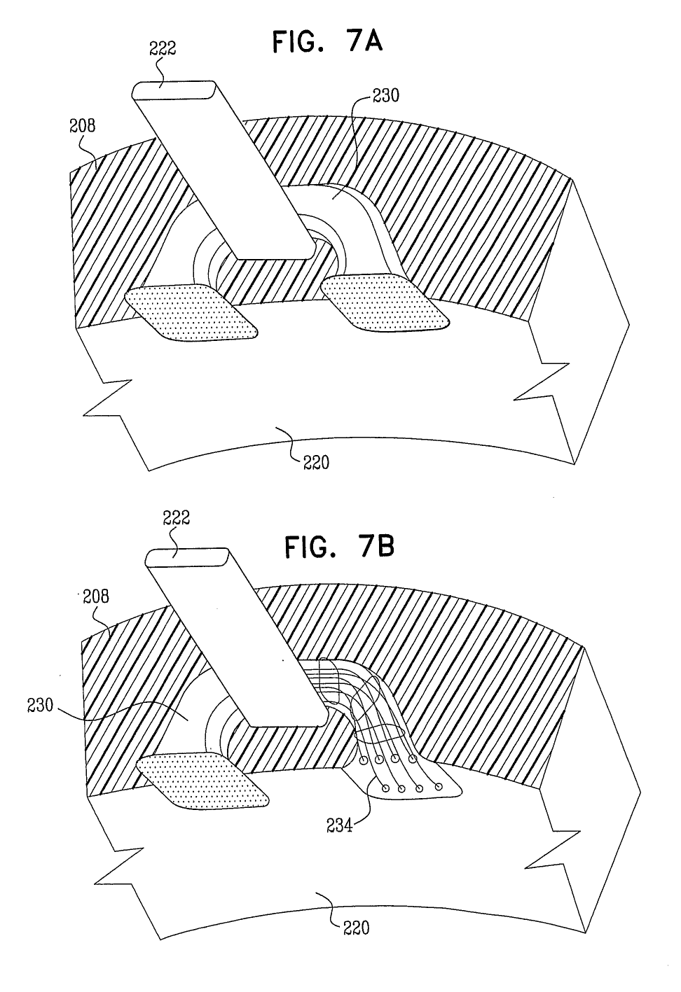

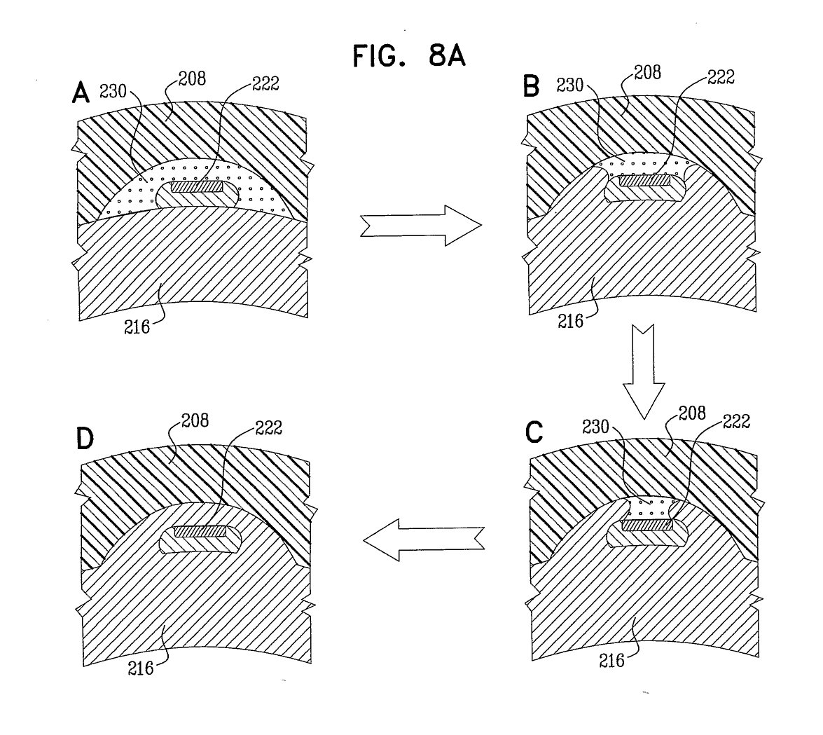

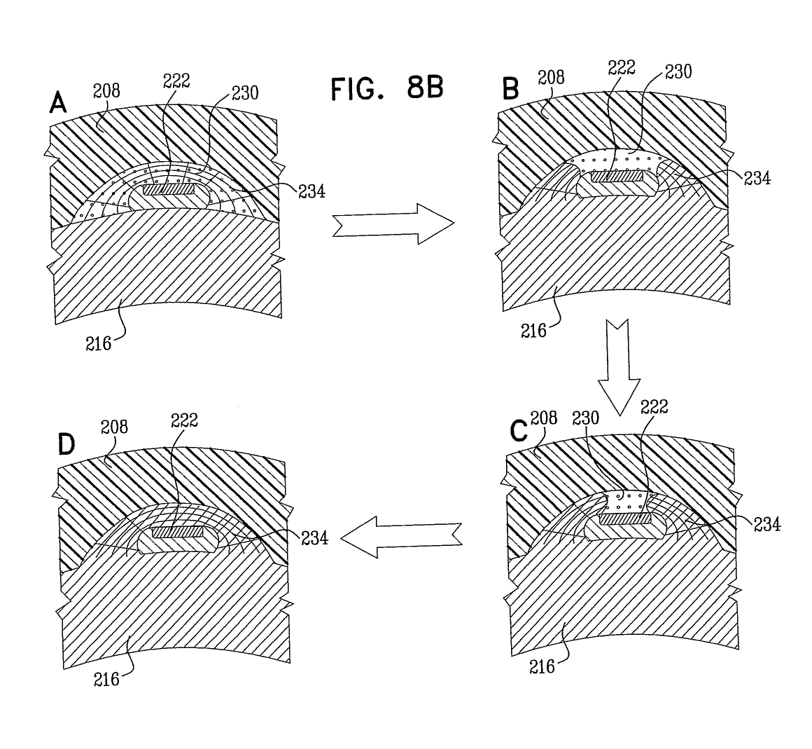

Reference is now made to FIG. 6, which is a simplified anatomical illustration showing various applications of a grown bone locked splint implant constructed and operative in accordance with a preferred embodiment of the present invention, to FIGS. 7A & 7B, which are simplified partial illustrations of a plurality of alternative structures of grown bone lockable implant useful in the embodiment of FIG. 6, and to FIGS. 8A & 8B, which are simplified illustrations of sequential bone growth in the embodiments illustrated respectively in FIGS. 7A & 7B.

There is provided in accordance with a preferred embodiment of the present invention a splint implant including a splint implant element adapted to be attached to two portions of at least one bone, the implant element including a plurality of throughgoing bone growth regions adapted to be located in bone growth communication with the portions of the bone and configured such that bone growth therein creates a grown bone suture binding the implant element to the portions of the bone.

Additionally or alternatively, the splint implant element may be resilient and may be adapted to be attached to portions of a bone and may include a plurality of bone growth regions adapted to be located in bone growth communication with the portions of the bone and configured such that bone growth therein creates a grown bone lock binding the resilient implant element to the bone.

Additionally or alternatively, the splint implant element may be adapted to be attached to portions of a bone and may include a plurality of bone growth regions adapted to be located in bone growth communication with the bone portions and containing grown bone enhancing material configured such that bone growth therein creates a grown bone lock binding the splint implant element to the bone.

The grown bone enhancing material preferably comprises bone growth enhancing material and/or grown bone reinforcing material.

Turning initially to FIG. 6, there is provided a simplified anatomical illustration showing various applications of a grown bone locked splint implant of the type described hereinabove, constructed and operative in accordance with a preferred embodiment of the present invention.

A humerus splint implant assembly, shown in an enlargement designated by reference numeral 202, includes a grown bone locked humerus splint implant element 204 which is constructed and operative in accordance with a preferred embodiment of the present invention and arranged to surround portions of the humerus.

A femur splint implant assembly, shown in an enlargement designated by reference numeral 206, includes grown bone locked femur splint implant element 208, constructed and operative in accordance with a preferred embodiment of the present invention.

A tibia splint implant assembly, shown in an enlargement designated by reference numeral 212, includes a grown bone locked tibia splint implant element 214 constructed and operative in accordance with a preferred embodiment of the present invention.

Reference is now made to FIGS. 7A & 7B, which are simplified partial illustrations of a plurality of alternative structures of grown bone lockable splint implant useful in the embodiment of FIG. 6 and to FIGS. 8A & 8B, which are simplified illustrations of sequential bone growth in the embodiments illustrated respectively in FIGS. 7A & 7B.

FIGS. 7A & 7B represent two alternative embodiments of an exemplary implant element useful in a femur splint implant assembly as shown in enlargement 206 of FIG. 6. FIGS. 7A & 7B are partially pictorial partially sectional illustrations of implant element 208 taken generally along lines VII-VII in enlargement 206. FIGS. 8A & 8B are sectional illustrations of implant element 208 taken generally along the lines VII-VII in enlargement 206. It is appreciated that the descriptions of FIGS. 7A-8B which follow are applicable to any other suitable type of grown bone lockable implant.

Turning now to FIGS. 7A & 7B, there are seen partial illustrations of a portion of a grown bone lockable splint implant element 208, such as a femur splint implant element. FIG. 7A shows a portion of the implant element 208 arranged to surround portions 216 and 218 (FIG. 6) of an elongate bone, such as the femur. In the illustrated embodiment, the implant element is typically in the form of a split cylinder 220 formed of a somewhat resilient material, such as polyurethane, and is preferably formed with a plurality of elongate reinforcing strips 222, typically formed of a generally non-stretchable material such as a composite material or metal, embedded therein.

In accordance with a preferred embodiment of the present invention, the split cylinder 220 has formed therein a plurality of bone growth regions 230, which are preferably throughgoing, preferably are in the shape of a hook or a handle, and which preferably encircle one of the reinforcing strips 222 as shown. In accordance with a preferred embodiment of the present invention, bone growth regions 230 are generally filled with a resorbable material such as any one of the materials listed above with reference to FIG. 2A.

Additionally or alternatively, bone growth regions 230 are generally filled with a material, such as any one of the materials listed hereinabove with reference to FIGS. 2A & 2B.

FIG. 7B shows a portion of the implant element 208 having formed therein preferably throughgoing bone growth regions 230, preferably in the shape of a hook or a handle, as shown. In accordance with a preferred embodiment of the present invention, bone growth regions 230 are generally filed with a resorbable material of the type described hereinabove with reference to FIG. 7A. In addition, the splint implant element of FIG. 7B includes reinforcement elements 234, preferably relatively rigid elongate metal elements or relatively flexible fibers located in the bone growth regions 230, which preferably encircle a reinforcing strip 222.

Reference is now made to FIGS. 8A & 8B, which are simplified illustrations of sequential bone growth in the embodiments illustrated respectively in FIGS. 7A & 7B. FIGS. 8A and 8B each show at step A the implant element 208 positioned in contact with portions 216 and 218 (FIG. 6) of an elongate bone, such as the femur. The bone growth regions 230 are shown filled with one or more of the materials described hereinabove with reference to FIG. 2A. As seen in step A, bone growth has not yet commenced. At steps B and C respectively, initial and further bone growth from portions 216 and 218 (FIG. 6) of the femur into bone growth regions 230 are shown. Step D shows complete bone growth throughout the throughgoing bone growth regions 230. The entire bone growth process can be expected to take place over a period of a few months.

It is appreciated that the splint implant described hereinabove with reference to FIGS. 6-8B may be employed for broken elongated bones as well as for weakened bones, such as bones suffering from osteoporosis or bones weakened by cancer.

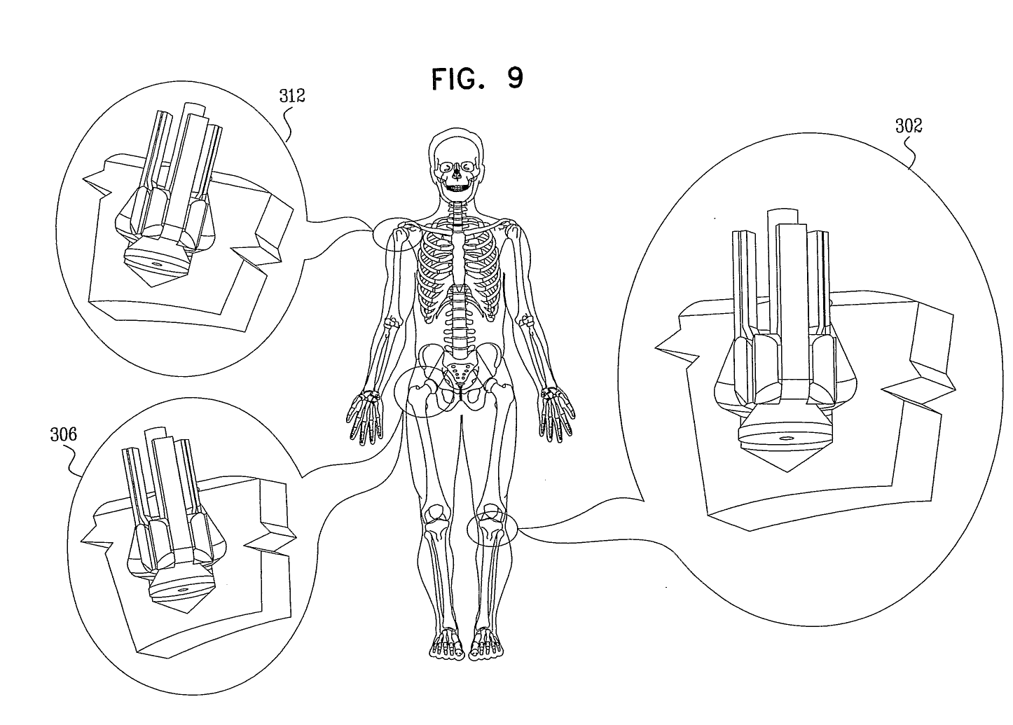

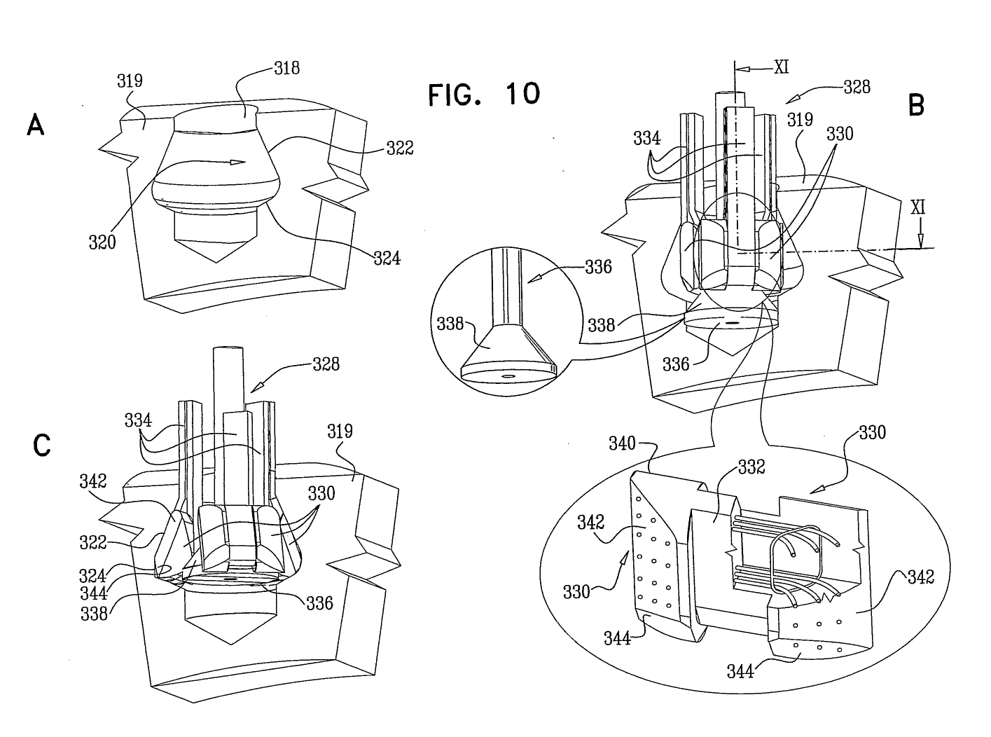

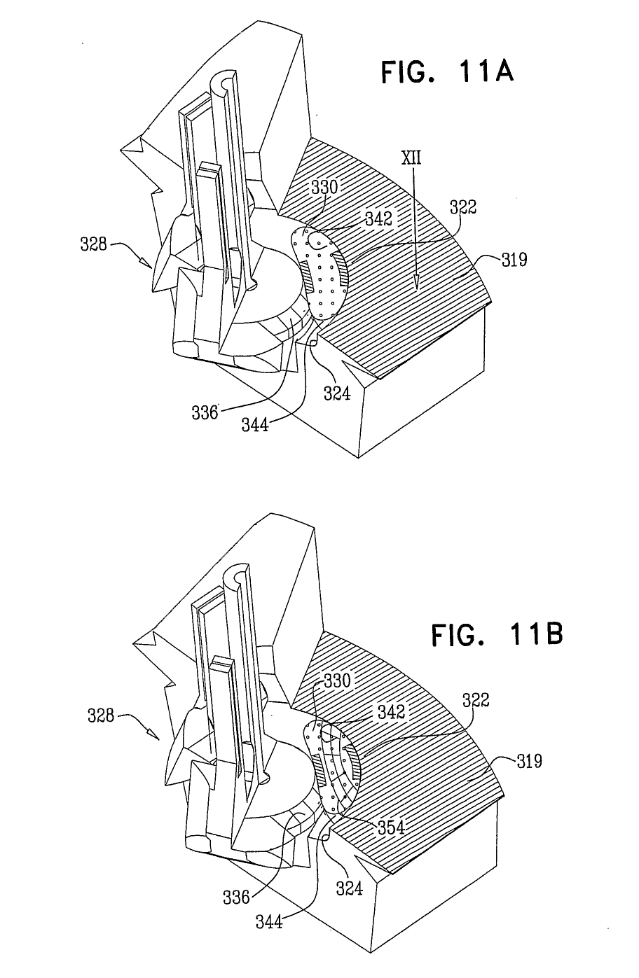

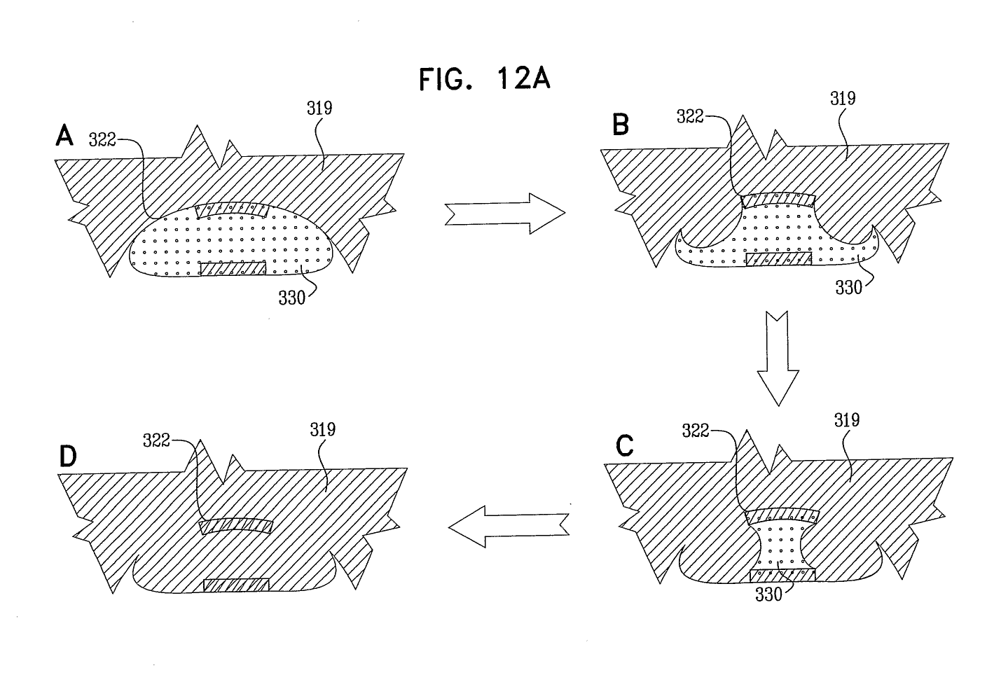

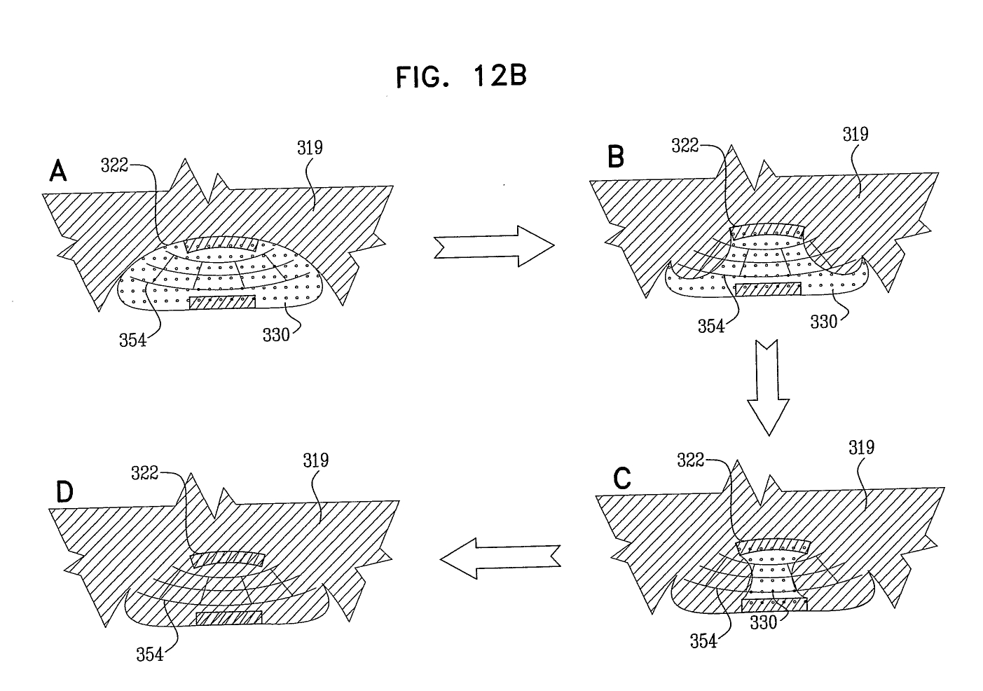

Reference is now made to FIG. 9, which is a simplified anatomical illustration showing various applications of a grown bone locked ligament attachment implant constructed and operative in accordance with a preferred embodiment of the present invention, to FIG. 10, which illustrates steps in the insertion of the ligament attachment implant, to FIGS. 11A & 11B which are simplified partial illustrations of a plurality of alternative structures of grown bone lockable implant useful in the embodiment of FIG. 9, and to FIGS. 12A & 12B, which are simplified illustrations of sequential bone growth in the embodiments illustrated respectively in FIGS. 10A & 10B.

There is provided in accordance with a preferred embodiment of the present invention a ligament attachment implant including a ligament attachment assembly comprising one or more ligament attachment elements adapted to be attached to a bone, each ligament attachment implant element adapted to be located in bone growth communication with a portion of the bone and configured such that bone growth therein creates a grown bone ligament anchor. Preferably, the ligament attachment elements contain grown bone enhancing material configured such that bone growth therein creates a grown bone lock binding the implant element to the bone.

The grown bone enhancing material preferably comprises bone growth enhancing material and/or grown bone reinforcing material.

Turning initially to FIG. 9, there is provided a simplified anatomical illustration showing various applications of a grown bone locked implant of the type described hereinabove, constructed and operative in accordance with a preferred embodiment of the present invention.

A knee ligament attachment assembly, constructed and operative in accordance with a preferred embodiment of the present invention, is shown in an enlargement designated by reference numeral 302.

A hip ligament attachment assembly, constructed and operative in accordance with a preferred embodiment of the present invention, is shown in an enlargement designated by reference numeral 306.

A shoulder ligament attachment assembly, constructed and operative in accordance with a preferred embodiment of the present invention, is shown in an enlargement designated by reference numeral 312.

Reference is now made to FIG. 10, which shows steps in the insertion of knee ligament attachment assembly in a tibia. It is appreciated that the hip and shoulder ligament attachment implant assemblies are similar in all relevant respects to the knee ligament attachment assembly shown in FIGS. 10-12B.

Initially, as shown at A, a hole 318 is formed in the tibia 319 by conventional surgical drilling techniques. The hole 318 is characterized in that it has a circumferential undercut, indicated generally by reference numeral 320. The undercut has a first circumferential surface 322 and therebelow a second circumferential surface 324. Generally, surfaces 322 and 324 are smoothly joined to define the undercut 320.

As seen at B, there is provided a knee ligament attachment assembly 328, which preferably comprises a plurality of ligament attachment elements 330, each having looped therearound, at a ligament loop retaining location 332 thereof, a closed looped ligament end 334. The closed loop ligament ends 334 may be formed of natural or artificial ligaments or portions thereof. A single ligament may be formed with multiple ligament ends. Ligament attachment elements 330 are preferably formed of or include bone growth enhancement materials, such as those referenced hereinabove in connection with FIG. 2A. In the illustrated embodiment, the knee ligament attachment assembly includes three ligament attachment elements 330.

In accordance with a preferred embodiment of the invention, each ligament attachment element 330 is adapted to be located in bone growth communication with a bone surface and configured such that bone growth therein creates a grown bone ligament anchor. Preferably, the bone growth enhancing material in the ligament attachment elements creates a grown bone lock binding the ligament attachment element to the bone.

Knee ligament attachment assembly 328 also includes a ligament attachment element positioner 336, preferably in the form of an elongate element formed with a generally conical surface 338 at an inner end thereof. Positioner 336 preferably has two operative orientations, a first, shown at B, where the generally conical surface is generally decoupled from attachment elements 330 and allows them to lie at a relatively radially inward position for insertion into hole 318.

A second operative orientation of positioner 336, shown at C, in which the generally conical surface 338 engages inner surfaces 340 of attachment elements 330, forces the attachment elements radially outwardly such that respective surfaces 342 of attachment elements 330 engage first circumferential bone surface 322 of undercut 320 and respective surfaces 344 of attachment elements 330 engage second circumferential bone surface 324 of undercut 320.

Following insertion of implant assembly 328 and positioning positioner 336 in its second operative orientation as shown at C, bone growth is allowed to take place while attachment elements 330 are retained such that respective surfaces 342 of attachment elements 330 engage first circumferential bone surface 322 of undercut 320 and respective surfaces 344 of attachment elements 330 engage second circumferential bone surface 324 of undercut 320.

Preferably, the bone growth enhancing material in the ligament attachment elements 330 creates a grown bone lock binding the implant element to the bone, thus creating a grown bone ligament anchor in accordance with a preferred embodiment of the present invention.

Reference is now made to FIGS. 11A & 11B, which are simplified partial illustrations of two alternative structures of grown bone lockable ligament attachment implant useful in the embodiment of FIG. 9 and to FIGS. 12A & 12B, which are simplified illustrations of sequential bone growth in the embodiments illustrated respectively in FIGS. 11A & 11B.

FIGS. 11A & 11B illustrate a portion of an exemplary ligament attachment assembly 328 as shown in enlargement 302 of FIG. 9. FIGS. 11A & 11B are pictorial illustrations of two alternative embodiments of ligament attachment assembly 328 taken generally along lines XI-XI in FIG. 10. FIGS. 12A & 12B are sectional illustrations of ligament attachment assembly 328 taken generally along lines XII-XII in FIG. 11A. It is appreciated that the descriptions of FIGS. 11A-12B which follow are applicable to any other suitable type of grown bone lockable implant.

Turning now to FIGS. 11A & 11B, there are seen partial illustrations of a portion of a grown bone lockable ligament attachment assembly 328. FIG. 11A shows a portion of the ligament attachment assembly 328 with positioner 336 in its second operative orientation such that bone growth is allowed to take place while ligament attachment elements 330 are retained such that respective surfaces 342 of ligament attachment elements 330 engage first circumferential bone surface 322 of undercut 320 and respective surfaces 344 of ligament attachment elements 330 engage second circumferential bone surface 324 of undercut 320.

FIG. 11B shows a portion of the ligament attachment assembly 328 including ligament attachment elements which also include reinforcement elements 354, preferably relatively rigid elongate metal elements or relatively flexible fibers.

Reference is now made to FIGS. 12A & 12B, which are simplified illustrations of sequential bone growth in the embodiments illustrated respectively in FIGS. 11A & 11B. FIGS. 12A and 12B each show at step A an ligament attachment element 330 positioned in contact with surface 322 of the tibia 319. As seen in step A, bone growth has not yet commenced. At steps B and C respectively, initial and further bone growth from surface 322 of the tibia 319 into the ligament attachment element 330 are shown. Step D shows complete bone growth throughout the ligament attachment element 330. The entire bone growth process can be expected to take place over a period of a few months.

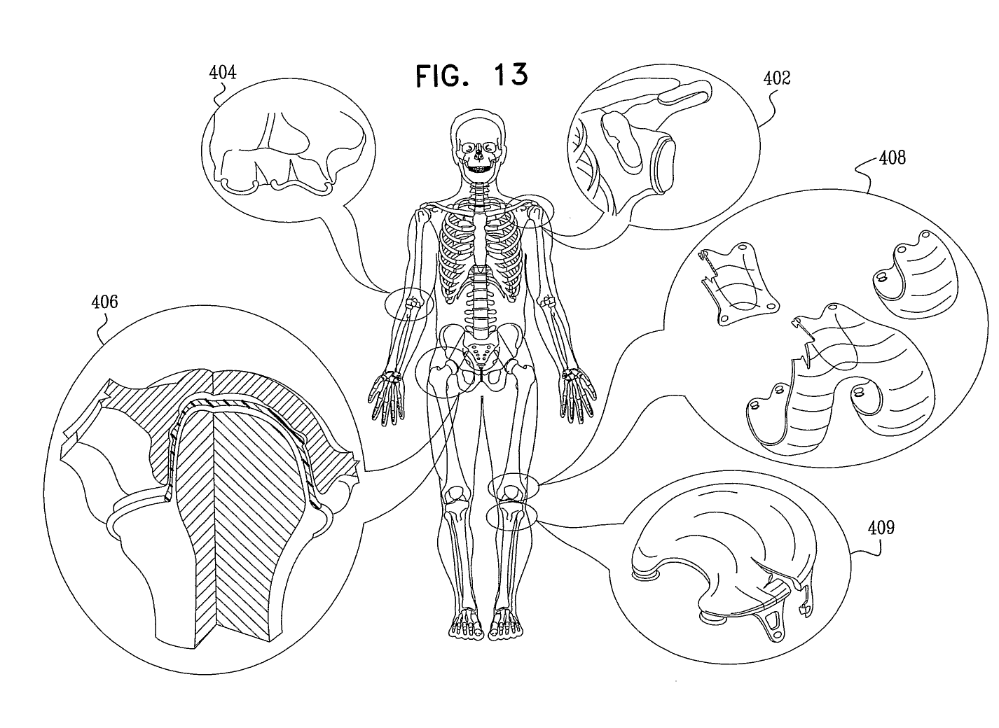

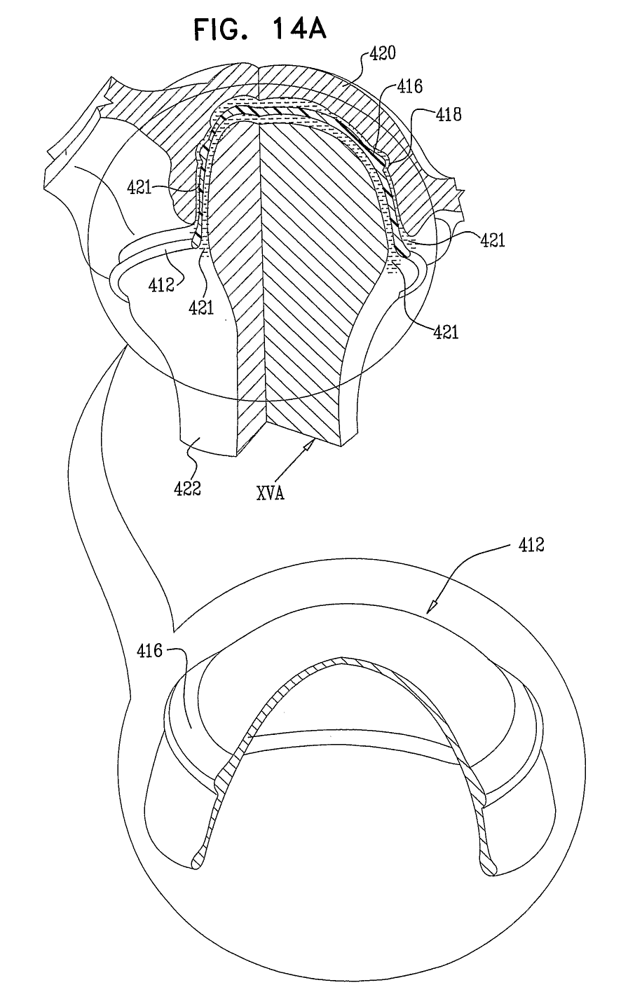

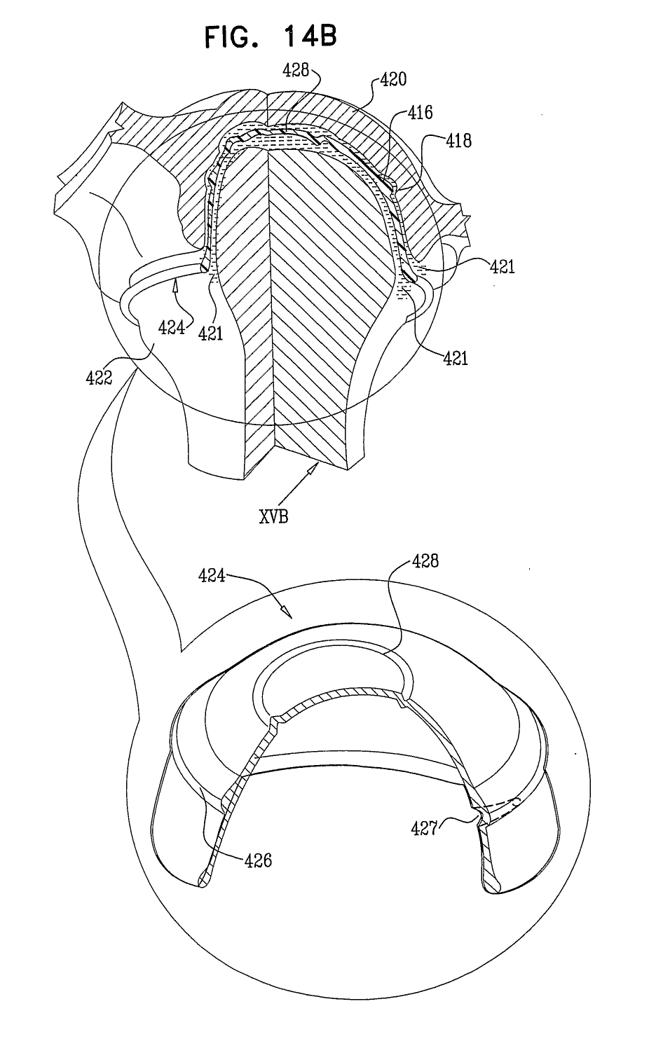

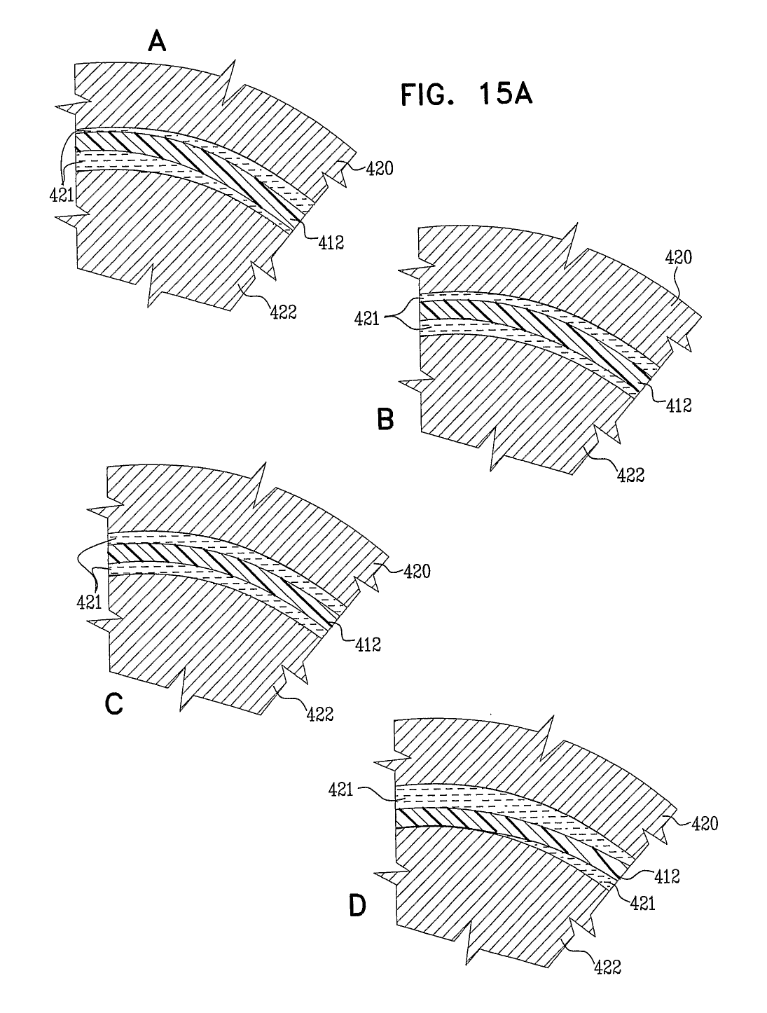

Reference is now made to FIG. 13, which is a simplified anatomical illustration showing various applications of an inter-surface articulating joint implant constructed and operative in accordance with a preferred embodiment of the present invention, to FIGS. 14A & 14B, which are simplified partial illustrations of a plurality of alternative structures of an inter-surface articulating joint implant useful in the embodiment of FIG. 13 and to FIGS. 15A & 15B, which are simplified illustrations of an aspect of the functionality of the inter-surface articulating joint implant in the embodiments illustrated respectively in FIGS. 14A & 14B.

There is provided in accordance with a preferred embodiment of the present invention an inter-surface articulating joint implant adapted to be located between articulating surfaces of a joint, the inter-surface articulating joint implant being a generally thin element formed of a flexible resilient material and being adapted to be generally surrounded by synovial fluid.

Preferably, the inter-surface articulating joint implant is formed with one or more throughgoing channels for passage of synovial fluid therethrough in response to changes in fluid pressure resulting from joint articulation and the application of changing forces to the joint.

The inter-surface articulating joint implant preferably serves to decrease or eliminate frictional engagement between the articulating surfaces of the joint. Additionally or alternatively, the inter-surface articulating joint implant may serve to enhance desired cartilage regeneration along one or both articulating surfaces of the joint.

Turning initially to FIG. 13, there is provided a simplified anatomical illustration showing various applications of an inter-surface articulating joint implant of the type described hereinabove, constructed and operative in accordance with a preferred embodiment of the present invention.

A shoulder inter-surface articulating joint implant, constructed and operative in accordance with a preferred embodiment of the present invention, is shown in an enlargement designated by reference numeral 402.

An elbow inter-surface articulating joint implant, constructed and operative in accordance with a preferred embodiment of the present invention, is shown in an enlargement designated by reference numeral 404.

A hip inter-surface articulating joint implant, constructed and operative in accordance with a preferred embodiment of the present invention, is shown in an enlargement designated by reference numeral 406.

A plurality of femur-patella/knee inter-surface articulating joint implants, constructed and operative in accordance with a preferred embodiment of the present invention, is shown in an enlargement designated by reference numeral 408.

A tibia/knee inter-surface articulating joint implant, constructed and operative in accordance with a preferred embodiment of the present invention, is shown in an enlargement designated by reference numeral 409.

Reference is now made to FIGS. 14A & 14B, which are simplified partial illustrations of a plurality of alternative structures of a hip inter-surface articulating joint implant, constructed and operative in accordance with a preferred embodiment of the present invention, useful in the embodiment of FIG. 13 and to FIGS. 15A & 15B, which are simplified illustrations of operation of the inter-surface articulating joint implant in the embodiments illustrated respectively in FIGS. 14A & 14B.

FIGS. 14A & 14B represent two alternative embodiments of an hip inter-surface articulating joint implant, constructed and operative in accordance with a preferred embodiment of the present invention, useful in the embodiment of FIG. 13. FIGS. 14A & 14B are pictorial illustrations of a hip inter-surface articulating joint implant 412. FIGS. 15A & 15B are sectional illustrations of hip inter-surface articulating joint implant 412, taken generally along lines XVA-XVA in FIG. 14A and lines XVB-XVB in FIG. 14B, respectively. It is appreciated that the descriptions of FIGS. 14A-15B which follow are applicable to any other suitable similar type of implant.

Turning now to FIGS. 14A & 14B, there are seen partial illustrations of a portion of hip inter-surface articulating joint implant 412, constructed and operative in accordance with a preferred embodiment of the present invention. FIG. 14A shows the implant 412 as a generally cup-shaped element having an outer facing circumferential protrusion 416 arranged to be loosely seated in a corresponding recess 418 in a surgically reamed acetabulum socket 420, with synovial fluid 421 being interposed between implant 412 and the surgically reamed acetabulum socket 420 and between implant 412 and a corresponding femoral head 422. In the illustrated embodiment, the implant 412 is preferably formed of a somewhat resilient material, such as polyurethane.

FIG. 14B shows a portion of an alternative embodiment of a hip inter-surface articulating joint implant, here designated by reference numeral 424, the structure of which differs from the structure of implant 412 (FIG. 14A) in that throughgoing respective non-inclined and inclined passageways 426 and 427 are formed to allow synovial fluid 421 to pass through the implant and the implant is formed with a relatively thin and highly resilient membrane portion 428, preferably arranged to be positioned at the eskelsis. The membrane portion preferably functions as a synovial fluid pump in response to articulation of the hip joint and variations in synovial fluid pressure thereat, thereby enhancing synovial fluid flow through passageways 426. This reduces friction in the joint.

Reference is now made to FIGS. 15A & 15B, which are simplified illustrations of an aspect of the initial functionality of embodiments illustrated respectively in FIGS. 14A & 14B. FIG. 15A shows at A the implant 412 disposed in synovial fluid 421 between socket acetabulum socket 420 and femoral head 422, following surgical insertion. It is seen that the shape of the implant 412 does not necessarily conform to the corresponding shapes of the articulating surfaces of the acetabulum socket 420 and the femoral head 422.

Following at least some articulation of the hip joint, such as due to running or walking, as shown at B, the shape of implant 412 becomes more conformal to the corresponding shapes of acetabulum socket 420 and femoral head 422.

One preferred steady-state, long term arrangement of implant 412 is shown at C, wherein the shape of the implant 412 is highly conformal to the corresponding shapes of acetabulum socket 420 and femoral head 422 and the implant floats in the synovial fluid 421 between the articulating surfaces of the acetabulum socket 420 and the femoral head 422, as the result of equilibrium in the fluid dynamic pressures exerted on the implant 412 by the synovial fluid alongside both surfaces thereof.

Another preferred steady-state, long term arrangement of implant 412 is shown at D, wherein the shape of the implant 412 is somewhat less conformal to the corresponding shapes of acetabulum socket 420 and femoral head 422 and the implant partially floats in the synovial fluid 421 between the articulating surfaces of the acetabulum socket 420 and the femoral head 422, as the result of equilibrium in the fluid dynamic pressures exerted on the implant 412 by the synovial fluid alongside both surfaces thereof and partially contacts one or both of the acetabulum socket 420 and the femoral head 422. The operative states shown at C and D may each occur from time to time in a patient or alternatively either operative state may predominate.

FIG. 15B shows at A the implant 424 disposed in synovial fluid 421 between acetabulum socket 420 and femoral head 422, following surgical insertion. It is seen inclined passageway 427 provides synovial fluid communication between a first synovial fluid region, designated by reference numeral 430, between implant 424 and the surgically reamed acetabulum socket 420 and a second synovial fluid region 432 between implant 424 and a corresponding femoral head 422.

Following at least some articulation of the hip joint, such as due to running or walking, as shown at B, flow of synovial fluid, which may be enhanced by the action of the synovial fluid pump defined by membrane 428, through inclined passageways 427 causes micro-rotation of the implant 424, relative to the acetabulum socket 420, as indicated, for example, by an arrow 434. The micro-rotation tends to prevent the implant 424 from being frozen in position relative to either of the articulating surfaces of the joint, such as the acetabulum socket 420. The micro-rotation also contributes to lowering friction between the implant 424 and the articulating surfaces of the joint upon joint articulation.

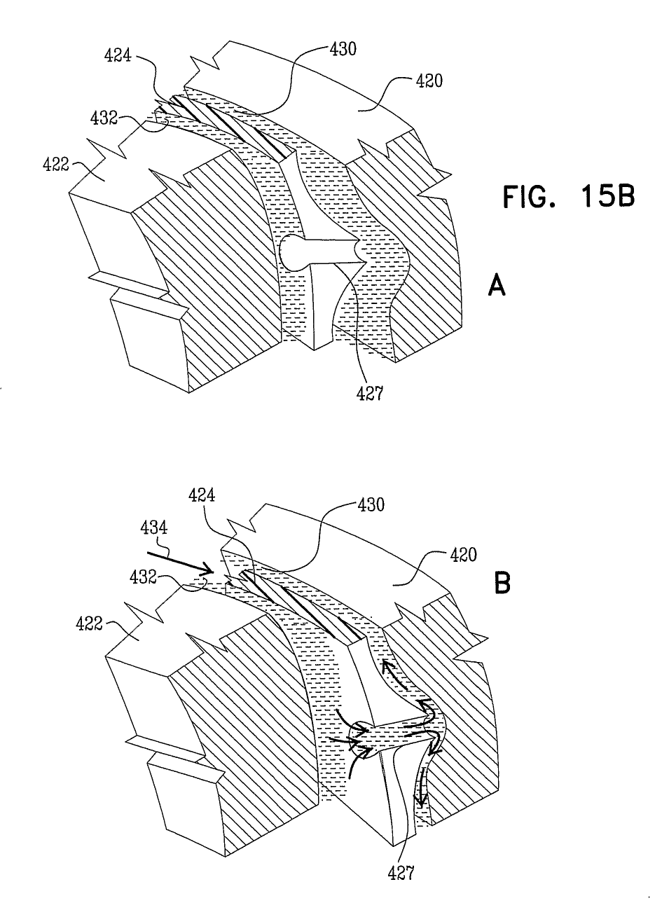

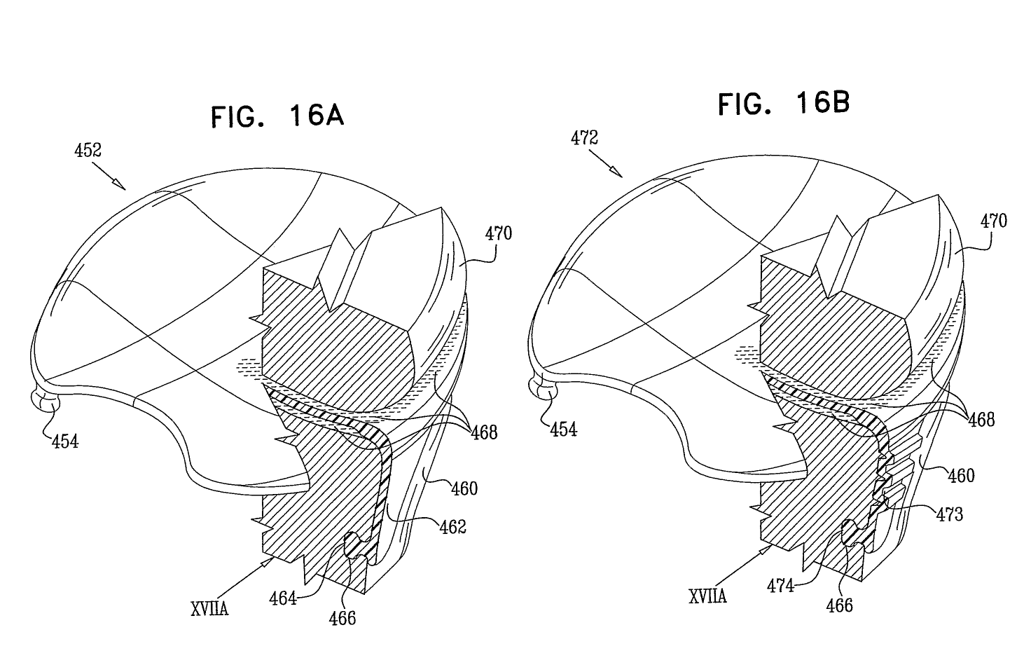

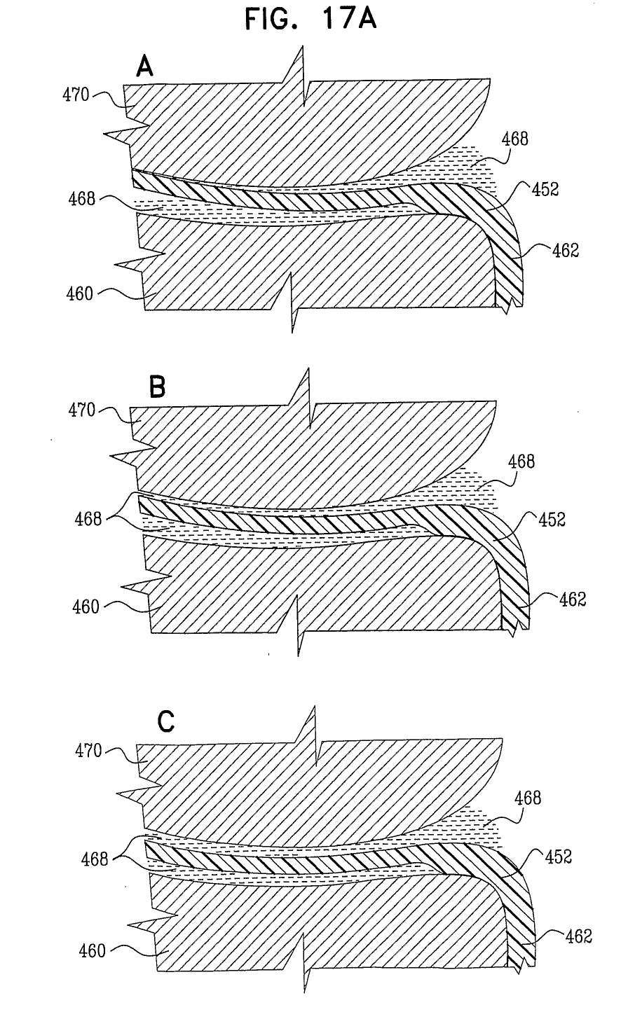

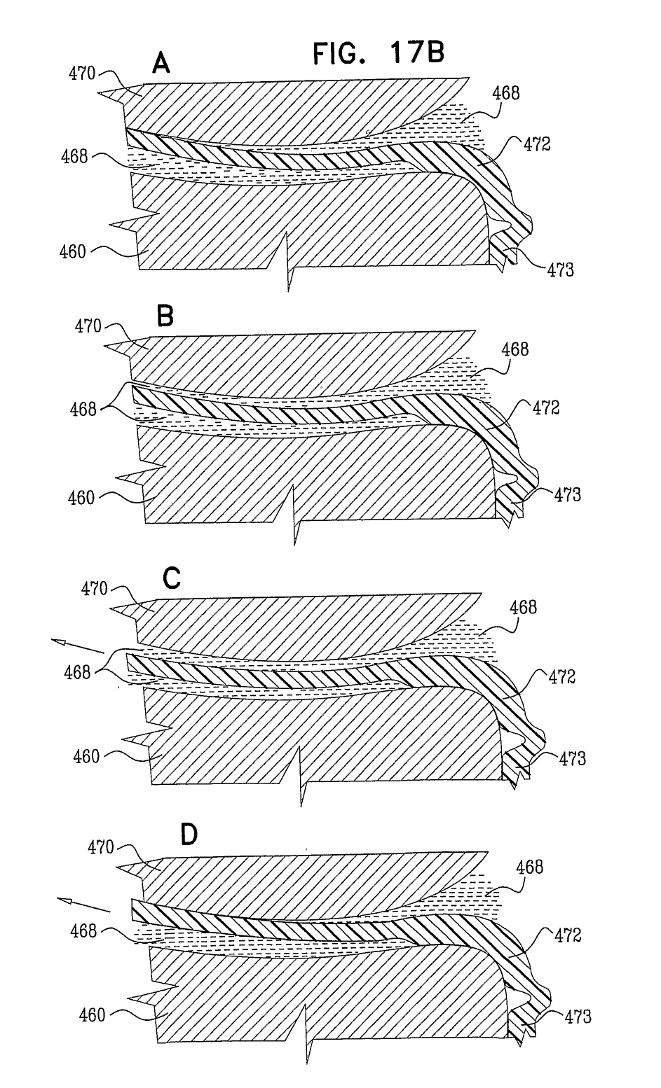

Reference is now made to FIGS. 16A & 16B, which are simplified partial illustrations of a plurality of alternative structures of a tibia/knee inter-surface articulating joint implant, constructed and operative in accordance with a preferred embodiment of the present invention, useful in the embodiment of FIG. 13 and to FIGS. 17A & 17B, which are simplified illustrations of operation of the inter-surface articulating joint implant in the embodiments illustrated respectively in FIGS. 16A & 16B. The inter-surface articulating joint implants of the present invention are preferably formed of a resilient material such as polyurethane but alternatively may be made of a material which is liquid absorbing, such as HydroThane™ which is commercially available from Cardiotech International Ct Biomaterials.

FIGS. 16A & 16B represent two alternative embodiments of a tibia/knee inter-surface articulating joint implant, constructed and operative in accordance with a preferred embodiment of the present invention, useful in the embodiment of FIG. 13. FIGS. 16A & 16B are pictorial illustrations of a tibia/knee inter-surface articulating joint implant 452, of the type seen in enlargement 409. FIGS. 17A & 17B are sectional illustrations of the tibia/knee inter-surface articulating joint implant 452, taken generally along lines XVIIA-XVIIA in FIG. 16A and lines XVIIB-XVIIB in FIG. 16B, respectively. It is appreciated that the descriptions of FIGS. 16A-17B which follow are applicable to any other suitable similar type of implant.

Turning now to FIGS. 16A & 16B, there are seen partial illustrations of a portion of the tibia/knee inter-surface articulating joint implant 452, constructed and operative in accordance with a preferred embodiment of the present invention. FIG. 16A shows the implant 452 as a generally flat element having at least one somewhat resilient protrusion 454 arranged to be tightly seated in a corresponding socket (not shown), surgically formed in the tibia 460, preferably in snap-fit engagement therewith. Implant 452 is also preferably formed with at least one tab 462 having a somewhat resilient protrusion 464 arranged to be tightly seated in a corresponding socket 466, surgically formed in the tibia 460, preferably in snap-fit engagement therewith.

Synovial fluid 468 is interposed between implant 452 and the tibia 460 and between implant 452 and a corresponding femoral condyle 470. In the illustrated embodiment, the implant 452 is preferably formed of a somewhat resilient material, such as polyurethane.

FIG. 16B shows a portion of an alternative embodiment of a tibia/knee inter-surface articulating joint implant, here designated by reference numeral 472, the structure of which differs from the structure of implant 472 in that tab 462 (FIG. 16A) is replaced by a resiliently extendible tab 473. Resiliently extendible tab 473 includes a somewhat resilient protrusion 474 arranged to be tightly seated in socket 466, surgically formed in the tibia 460, preferably in snap-fit engagement therewith.

Reference is now made to FIGS. 17A & 17B, which are simplified illustrations of an aspect of the functionality of embodiments illustrated respectively in FIGS. 16A & 16B. FIG. 17A shows at A the implant 452 disposed in synovial fluid 468 between tibia 460 and femur 470. It is seen that the shape of the implant 452 does not necessarily conform to the corresponding shapes of the articulating surfaces of the tibia 460 and the femur 470.

Following at least some articulation of the knee, such as due to running or walking, as shown at B, the shape of implant 452 becomes more conformal to the corresponding shapes of the tibia 460 and the femur 470. A preferred steady-state, long term arrangement of implant 452 is shown at C, wherein the shape of the implant 452 is highly conformal to the corresponding shapes of the tibia 460 and the femur 470 and the implant 452 floats in an anchored manner in the synovial fluid 468 between the articulating surfaces of the tibia 460 and the femur 470, as the result of equilibrium in the fluid dynamic pressures exerted on the implant 452 by the synovial fluid alongside both surfaces thereof. The anchoring of implant 452 by protrusion 454 (FIG. 16A) and by tab 462 (FIG. 16A) maintains desired positioning of the implant 452 in the knee.

FIG. 17B shows at A the implant 472 disposed in synovial fluid 468 between the tibia 460 and the femur 470. It is seen that the shape of the implant 472 does not necessarily conform to the corresponding shapes of the articulating surfaces of the tibia 460 and the femur 470.

Following at least some articulation of the knee, such as due to running or walking, as shown at B, the shape of implant 472 becomes more conformal to the corresponding shapes of the tibia 460 and the femur 470.

A preferred steady-state, long term arrangement of implant 472 is shown at C, wherein the shape of the implant 472 is highly conformal to the corresponding shapes of the tibia 460 and the femur 470 and the implant 472 floats in a flexible anchored manner in the synovial fluid 468 between the articulating surfaces of the tibia 460 and the femur 470, as the result of equilibrium in the fluid dynamic pressures exerted on the implant 452 by the synovial fluid alongside both surfaces thereof.

Another preferred steady-state, long term arrangement of implant 472 is shown at D, wherein the shape of the implant 472 is somewhat less conformal to the corresponding shapes of tibia 460 and femur 470 and the implant partially floats in the synovial fluid 468 between the articulating surfaces of the tibia 460 and the femur 470, as the result of equilibrium in the fluid dynamic pressures exerted on the implant 472 by the synovial fluid alongside both surfaces thereof and partially contacts one or both of the tibia 460 and the femur 470. The operative states shown at C and D may each occur from time to time in a patient or alternatively either operative state may predominate.

The flexible anchoring of implant 472 by protrusion 454 (FIG. 16B) and by resiliently extendible tab 473 maintains desired positioning of the implant 472 in the knee while allowing more lateral motion of the implant in the sense of FIGS. 17A and 17B than the arrangement shown in FIGS. 16A and 16B.

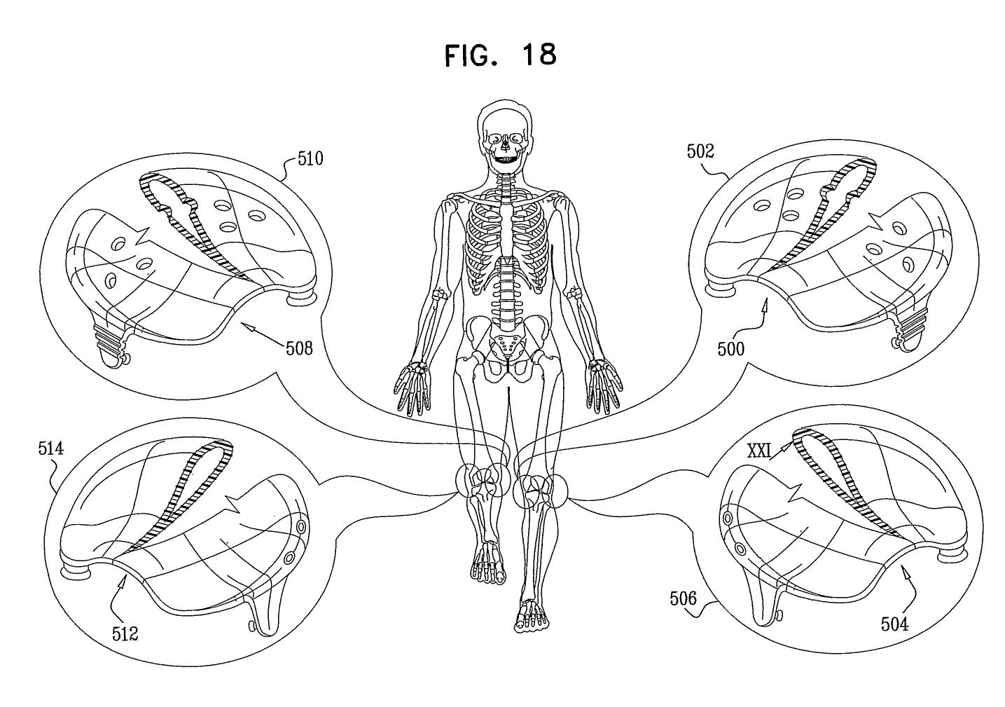

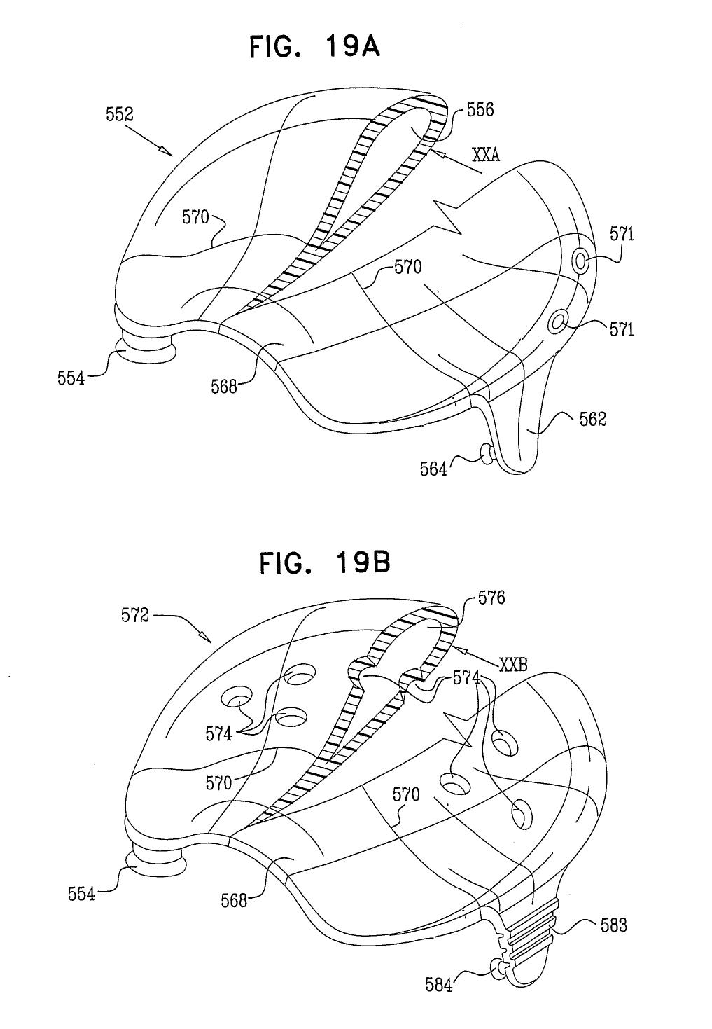

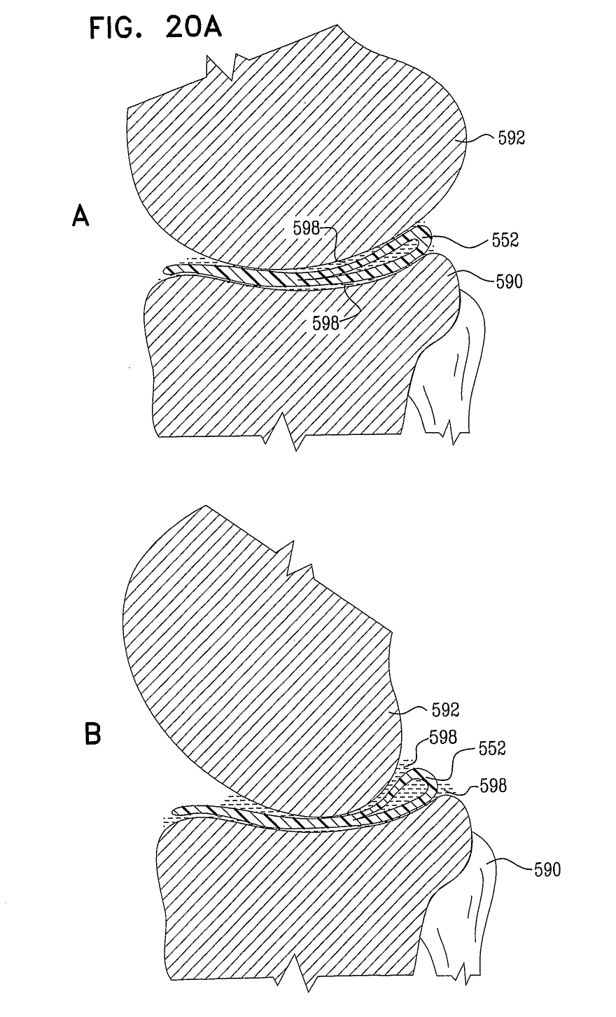

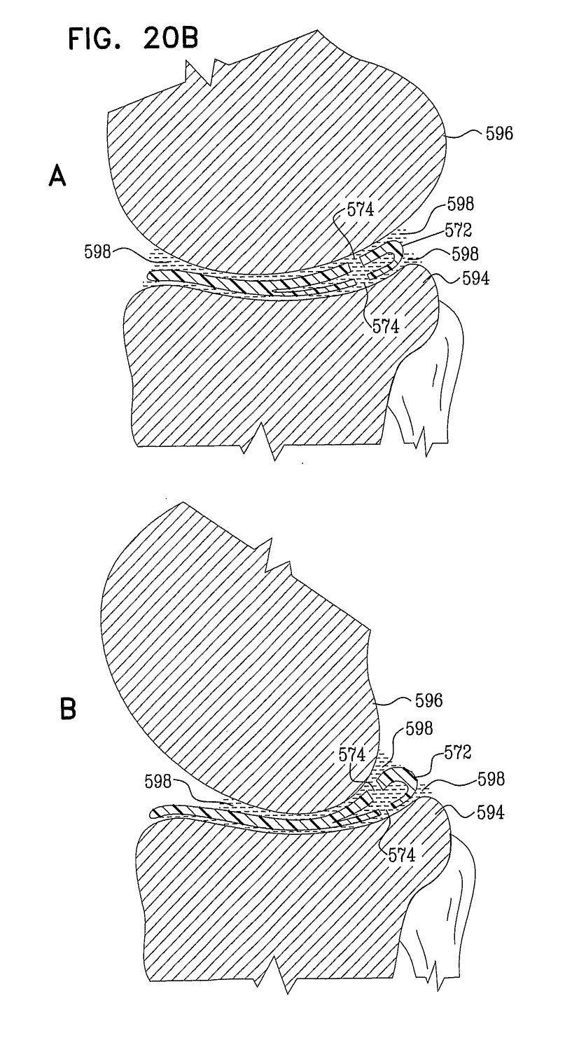

Reference is now made to FIG. 18, which is a simplified anatomical illustration showing various applications of a meniscus implant constructed and operative in accordance with a preferred embodiment of the present invention, to FIGS. 19A & 19B, which are simplified partial illustrations of a plurality of alternative structures of a meniscus implant useful in the embodiment of FIG. 18 and to FIGS. 20A & 20B, which are simplified illustrations of an aspect of the functionality of the meniscus implant in the embodiments illustrated respectively in FIGS. 19A & 19B.

There is provided in accordance with a preferred embodiment of the present invention a meniscus implant adapted to be located between articulating surfaces of a tibia and a femur, the meniscus implant being a generally thin element formed of a flexible resilient material and being adapted to be generally surrounded by synovial fluid.

Preferably, the meniscus implant is formed with one or more throughgoing channels for passage of synovial fluid therethrough in response to changes in fluid pressure resulting from knee joint articulation and the application of changing forces to the knee joint.

The meniscus implant preferably serves to decrease or eliminate frictional engagement between the articulating surfaces of the knee joint. Additionally or alternatively, articulation of the knee joint may serve to enhance desired cartilage regeneration along one or both articulating surfaces of the knee joint.

Turning initially to FIG. 18, there is provided a simplified anatomical illustration showing various applications of a meniscus implant of the type described hereinabove, constructed and operative in accordance with a preferred embodiment of the present invention. A left medial meniscus implant 500, constructed and operative in accordance with a preferred embodiment of the present invention, is shown in an enlargement designated by reference numeral 502 for a straight leg orientation. A left lateral meniscus implant 504, constructed and operative in accordance with a preferred embodiment of the present invention, is shown in an enlargement designated by reference numeral 506 for a straight leg orientation. A right medial meniscus implant 508, constructed and operative in accordance with a preferred embodiment of the present invention, is shown in an enlargement designated by reference numeral 510 for a bent knee orientation. A right lateral meniscus implant 512, constructed and operative in accordance with a preferred embodiment of the present invention, is shown in an enlargement designated by reference numeral 514 for a bent knee orientation.

Reference is now made to FIGS. 19A & 19B, which are simplified partial illustrations of a plurality of alternative structures of a medial meniscus implant, constructed and operative in accordance with a preferred embodiment of the present invention, useful in the embodiment of FIG. 18 and to FIGS. 20A & 20B, which are simplified illustrations of operation of the medial meniscus implant in the embodiments illustrated respectively in FIGS. 19A & 19B. The meniscus implants of the present invention are preferably formed of a resilient material such as polyurethane but alternatively made be made of a material which is liquid absorbing, such as HydroThane™ which is commercially available from Cardiotech International Ct Biomaterials.

FIGS. 19A & 19B represent two alternative embodiments of a meniscus implant, constructed and operative in accordance with a preferred embodiment of the present invention, useful in the embodiment of FIG. 18. FIGS. 19A and 19B are pictorial illustrations of two alternative embodiments of medial meniscus implant 500. FIGS. 20A and 20B each include sectional illustrations of meniscus implants 500 and 508, respectively taken generally along lines XXA-XXA in FIG. 19A and lines XXB-XXB in FIG. 19B.

Turning now to FIGS. 19A & 19B, there are seen partial illustrations of a portion of meniscus implant 552 of the type of meniscus implant 500, constructed and operative in accordance with a preferred embodiment of the present invention. FIG. 19A shows the implant 552 as a generally kidney-shaped, partially hollow generally flat element having at least one somewhat resilient protrusion 554 arranged to be tightly seated in a corresponding socket (not shown), surgically formed in the tibia, preferably in snap-fit engagement therewith. Implant 500 is also preferably formed with at least one tab 562 having a somewhat resilient protrusion 564 arranged to be tightly seated in a corresponding socket (not shown), surgically formed in the tibia, preferably in snap-fit engagement therewith.

As noted above, implant 500 is preferably partially hollow and preferably includes a hollow portion 566 which tapers down to a relatively thin non-hollow portion 568 along a transition line 570. Preferably at least one valve 571 is formed in hollow portion 566 to permit filling of the hollow portion 566 with a suitable fluid or gel, such as HydroSlip C which is commercially available from Cardiotech International Ct Biomaterials, Salubria which is commercially available from Salumedica, Llc of 112 Krog Street, Suite 4 Atlanta, Ga. or PuraMatrix which is commercially available from 3DM Inc. of Cambridge, Mass. As a further alternative, the hollow portion 566 may be filled with a self-polymerizing material such as in situ curable polyurethane which is commercially available from Advanced Bio Surfaces, Inc. of 5909 Baker Road, Suite 550, Minnetonka, Minn.

Alternatively at least one valve 571 may be obviated and the hollow portion 566 may be sealed and contain any suitable fluid. It is appreciated that the hollow portion 566 may be filled and sealed at the factory or at the time of implantation, such as in situ.

Synovial fluid is interposed between implant 552 and the tibia and between implant 552 and a corresponding femoral condyle.

FIG. 19B shows a portion of an alternative embodiment of a meniscus implant, here designated by reference numeral 572, the structure of which differs from the structure of implant 552, shown in FIG. 19A, in that apertures 574 are formed in the walls defining a hollow portion 576 to enable synovial fluid to readily communicate therethrough and to be pumped by changes in the volume of the interior of the hollow portion 576 resulting from articulation of the knee joint. Another feature shown in FIG. 19B, which feature may also be incorporated in the embodiment of FIG. 19A, is that tab 562 (FIG. 19A) is replaced by a resiliently extendible tab 583. Resiliently extendible tab 583 includes a somewhat resilient protrusion 584 arranged to be tightly seated in a corresponding socket (not shown), surgically formed in the tibia, preferably in snap-fit engagement therewith.

Reference is now made to FIGS. 20A & 20B, which are simplified illustrations of an aspect of the functionality of embodiments illustrated respectively in FIGS. 19A & 19B. FIG. 20A shows at A the implant 552 disposed between a tibia 590 and a femur 592. Synovial fluid 598 is interposed between implant 552 and the tibia 590 and between implant 552 and a corresponding femoral condyle. It is seen that at stage A, when the leg is relatively straight, as shown in enlargement 502 of FIG. 18, a relatively large percentage of the surface area of the implant is engaged by the tibia and the femur.

It is seen that at stage B, when the knee is bent, as shown in enlargement 510 of FIG. 18, a relatively small percentage of the surface area of the implant is engaged by the tibia 590 and the femur 592. It is appreciated that the implant shown at B is the right medial meniscus implant 508 (FIG. 18), while the implant shown at A is the left medial meniscus implant 500 (FIG. 18). It is appreciated that there exist an extremely large number of operative orientations of the meniscus implant which are not shown here for the sake of conciseness.

FIG. 20B shows at A the implant 572 (FIG. 19B) disposed between a tibia 594 and a femur 596. It is seen that at stage A, when the leg is relatively straight, as shown in enlargement 502 of FIG. 18, a relatively large percentage of the surface area of the implant is engaged by the tibia 594 and the femur 596.

It is appreciated that the implant shown at B is the right medial meniscus implant 508 (FIG. 18), while the implant shown at A is the left medial meniscus implant. It is appreciated that there exist an extremely large number of operative orientations of the meniscus implant which are not shown here for the sake of conciseness.

It is seen that at stage B, when the knee is bent, as shown in enlargement 510 of FIG. 18, a relatively small percentage of the surface area of the implant is engaged by the tibia 594 and the femur 596. It is noted that due to the pumping action produced by articulation of the knee joint on the apertured meniscus implant 572, a relatively large amount of synovial fluid 598 is provided in the vicinity of the meniscus implant 572 and between the meniscus implant and the corresponding articulating surfaces of the tibia 594 and the femur 596.

Preferably, the synovial fluid 598 is present along the walls of both the hollow and non-hollow portions of the meniscus implant 572.

It is a particular feature of the present invention that the structure of apertured, synovial fluid pumping implant 572 substantial reduces friction, increases lubrication and enhances the overall ease of knee joint articulation.

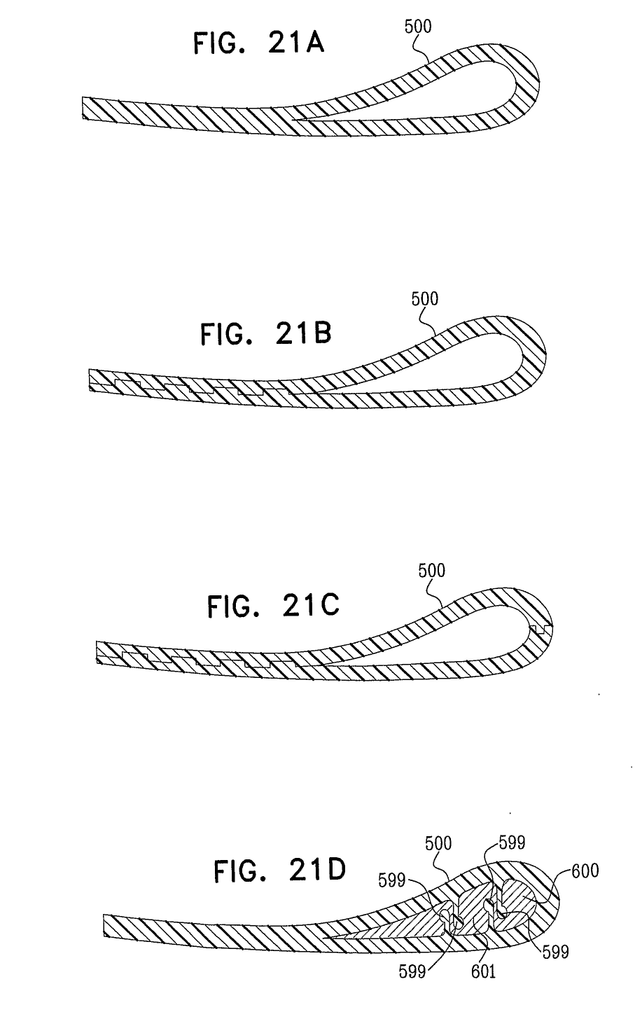

Reference is now made to FIGS. 21A, 21B, 21C and 21D, which are sectional illustrations of a plurality of alternative constructions of the meniscus implant of FIGS. 18-20B. FIG. 21A shows a unitary, integrally formed meniscus implant of the type shown in FIG. 18. This implant can be manufactured by injection molding. FIG. 21B shows an alternative embodiment of a meniscus implant of the type shown in FIG. 18 which comprises folded-over sheet material which is typically joined by press-fit joints or any other suitable bonding technique. FIG. 21C shows a further alternative embodiment of a meniscus implant of the type shown in FIG. 18 which comprises a pair of generally web-type elements which are joined along two seams by press-fit joints or any other suitable bonding technique.

FIG. 21D shows an embodiment of a meniscus implant of the type shown in FIG. 18 which comprises mutually engaging shape stabilizing internal structural elements 599 which cooperate with a polymerizeable material 600 in the interior of a hollow portion 601 to help maintain a desired three-dimensional shape of implant notwithstanding articulation of the knee joint. This structure is particularly useful for in-situ filling of the interior of the hollow portion 601.

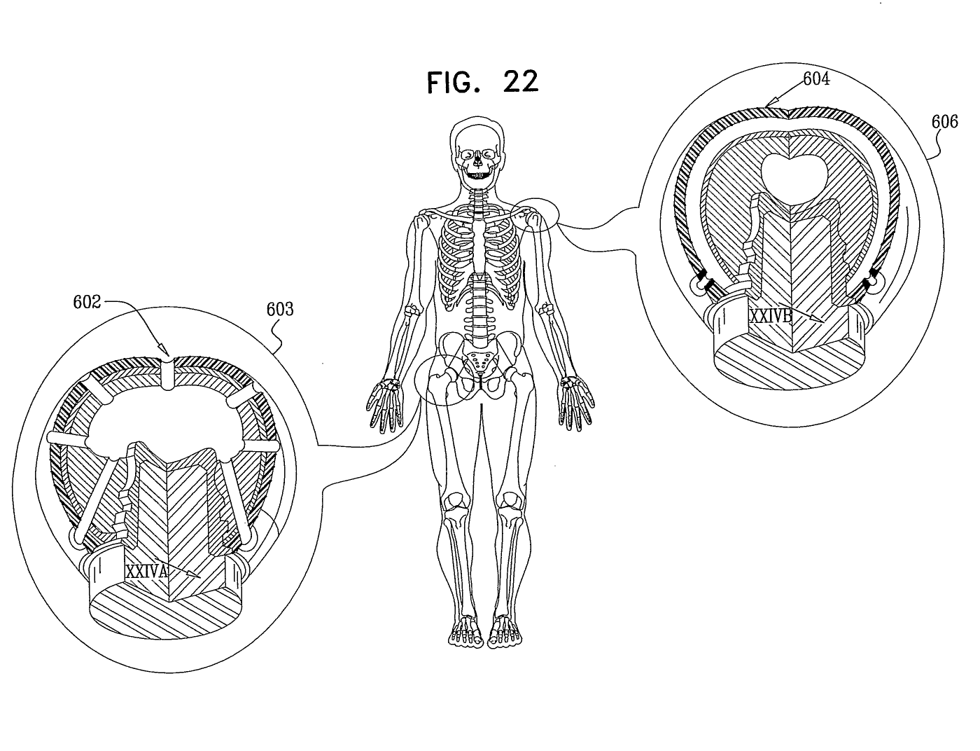

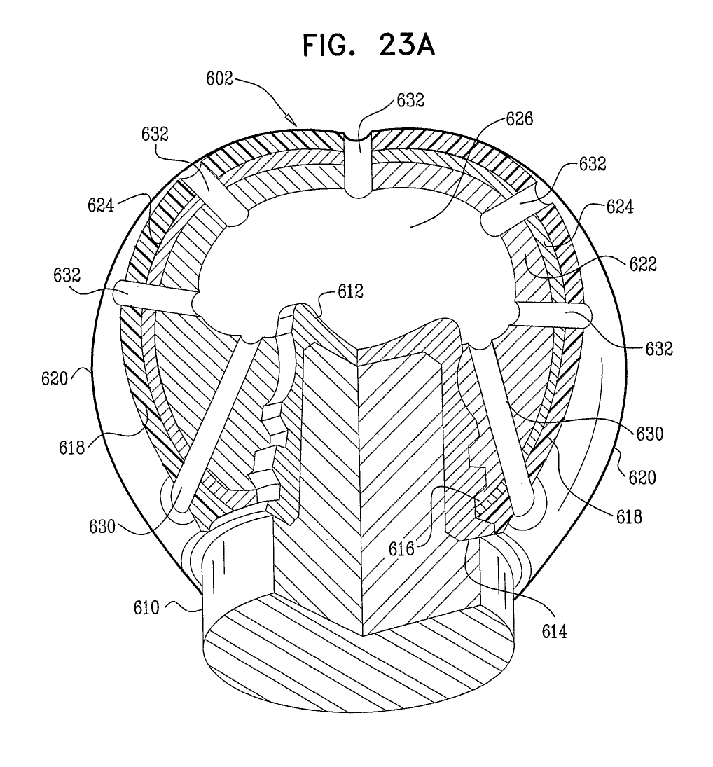

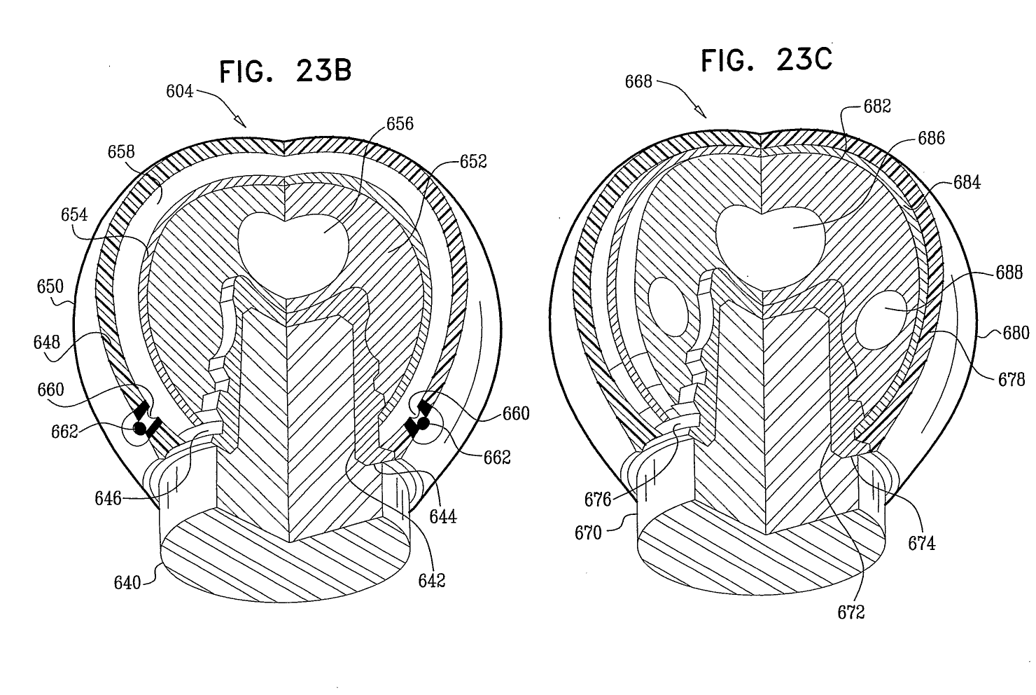

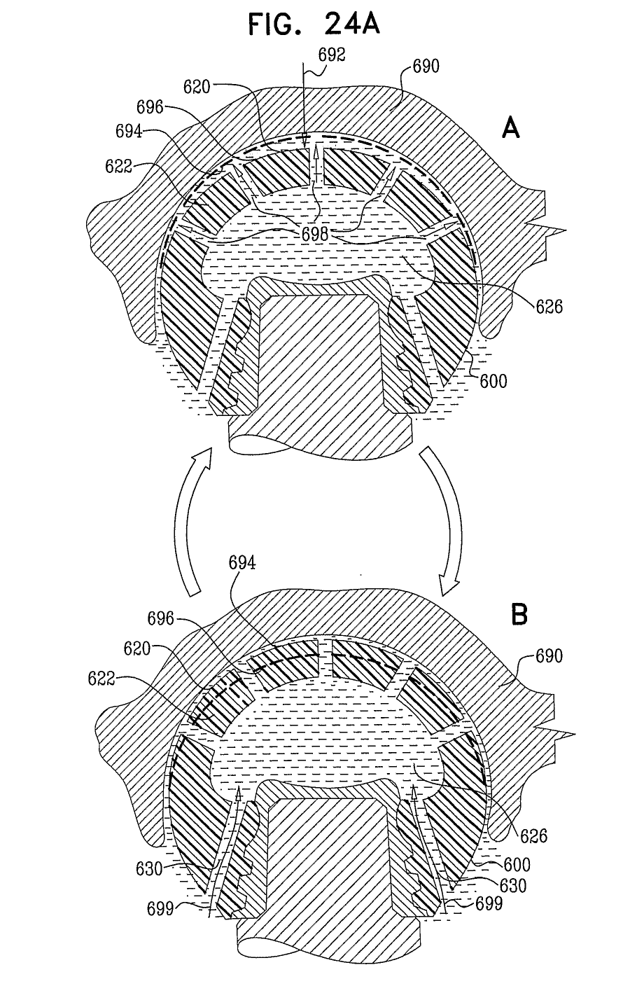

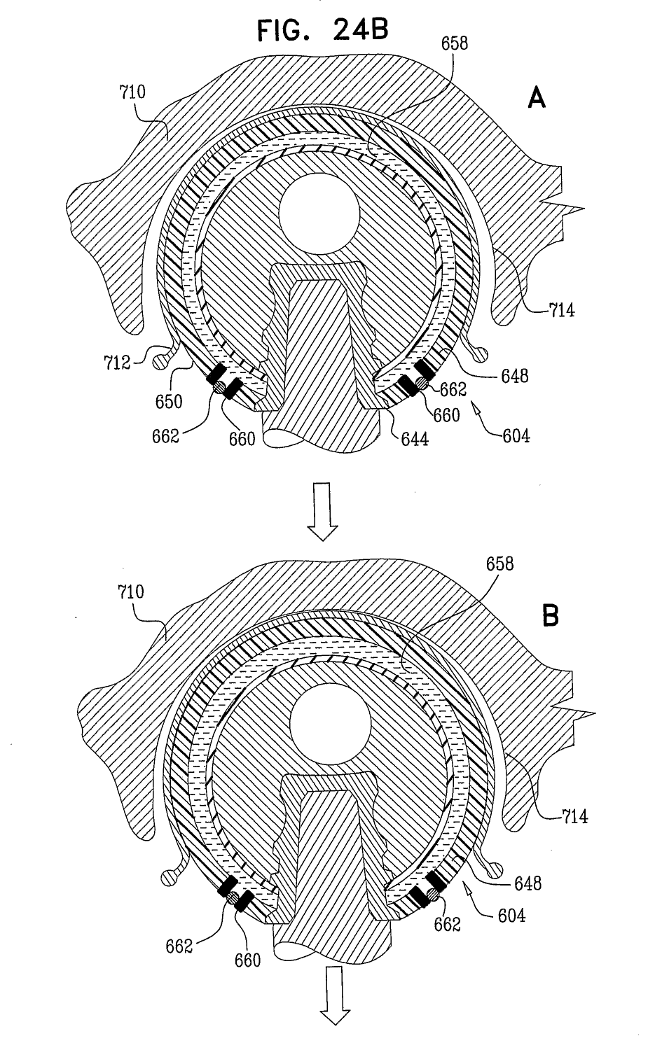

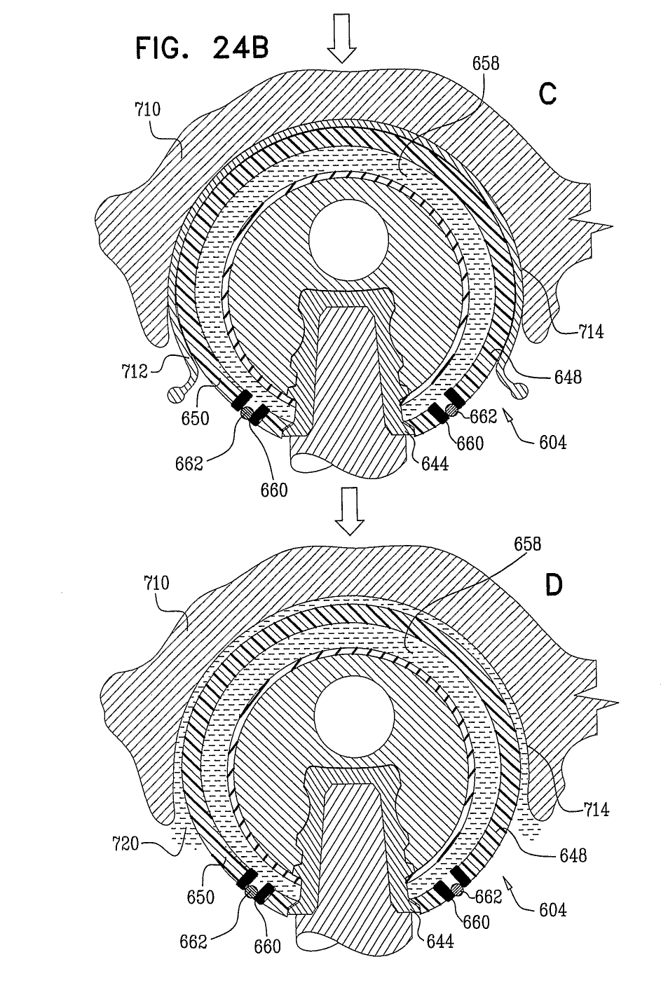

Reference is now made to FIG. 22, which is a simplified anatomical illustration showing various applications of a ball implant constructed and operative in accordance with a preferred embodiment of the present invention, to FIGS. 23A, 23B & 23C, which are simplified partial illustrations of a plurality of alternative structures of a ball implant useful in the embodiment of FIG. 22 and to FIGS. 24A & 24B, which are simplified illustrations of aspects of the functionality of the ball implants in the embodiments illustrated respectively in FIGS. 23A & 23B.

There is provided in accordance with a preferred embodiment of the present invention a ball implant adapted for articulation with an articulating socket of a joint, the ball implant being a shock-absorbing multi-layer assembly.

In accordance with one embodiment of the invention, the ball implant is formed with one or more throughgoing channels for passage of synovial fluid therethrough in response to changes in fluid pressure resulting from joint articulation and the application of changing forces to the joint.

In accordance with another embodiment of the present invention, a personalized fit, in situ configurable, ball implant is provided. Additionally or alternatively, articulation of the joint may serve to enhance desired cartilage regeneration along an articulating surface of a ball socket.

Turning initially to FIG. 22, there is provided a simplified anatomical illustration showing various applications of a ball implant of the type described hereinabove, constructed and operative in accordance with a preferred embodiment of the present invention. A hip joint ball implant 602, constructed and operative in accordance with a preferred embodiment of the present invention, is shown in an enlargement designated by reference numeral 603. A shoulder joint ball implant 604, constructed and operative in accordance with a preferred embodiment of the present invention, is shown in an enlargement designated by reference numeral 606.

Reference is now made to FIGS. 23A, 23B & 23C, which are simplified partial illustrations of a plurality of alternative structures of a ball implant, constructed and operative in accordance with a preferred embodiment of the present invention, useful in the embodiment of FIG. 22 and to FIGS. 24A & 24B, which are simplified illustrations of operation of the ball implant in the embodiments illustrated respectively in FIGS. 23A & 23B.

FIGS. 23A, 23B & 23C, present three alternative embodiments of a ball implant, constructed and operative in accordance with a preferred embodiment of the present invention, useful in the embodiment of FIG. 22. FIGS. 23A, 23B and 23C are pictorial illustrations of three alternative embodiments of ball implant 602. FIGS. 24A and 20B each include sectional illustrations of ball implants 602 and 604, respectively taken generally along respective lines XXIVA-XXIVA and XXIVB-XXIVB in enlargements 603 and 606.

Turning now to FIGS. 23A, 23B & 23C, there are seen partially cut-way illustrations of three alternative embodiments of a portion of ball implant 602, constructed and operative in accordance with a preferred embodiment of the present invention.

FIG. 23A shows the implant 602 as a generally ball-shaped, partially hollow multi-layer assembly arranged to be tightly seated onto a top portion of a conventional replacement femoral stem 610. The implant preferably comprises a replacement femoral stem socket 612, preferably formed of polyurethane having a Shore hardness designated as 70D and having an outer facing rim 614. Preferably mounted onto a circumferential recess 616 formed on an outer surface of socket 612 is a generally ball shaped hollow outer element 618, preferably formed of polyurethane having a Shore hardness designated as 80A and defining an articulating surface 620. Disposed within element 618 is a core element 622, preferably formed of foamed polyurethane of the same type used in element 618. Located between elements 618 and 622 is at least one relatively thin reinforcing layer 624, typically formed of KEVLAR® or carbon cloth. Core element 622 may be advantageously formed with at least one synovial fluid void 626.

At least one synovial fluid communication passageway 630 communicates with at least one void 626 at locations adjacent rim 614. A plurality of synovial fluid communication passageways 632 communicate between at least one void 626 through core element 622, at least one reinforcing layer 624 and element 618 with the articulating surface 620, thus providing synovial fluid communication with the articulating surface 620 of the implant. Preferably one-way flow valves (not shown) are associated with most or all of passageways 630 and 632. Pumping action produced by articulation of the ball implant 602 relative to an acetabulum socket (not shown) causes a relatively large amount of synovial fluid to be provided in the articulation region between the articulating surface 620 of the ball implant 602 and the corresponding articulating surface of the acetabulum socket (not shown). The pumping action preferably is constrained by the valves (not shown) such that synovial fluid flows towards the void 626 only via passageways 630 and from the void to the articulation region only via passageways 632.

FIG. 23B shows another embodiment of the implant 604 as a generally ball-shaped, partially hollow multi-layer assembly arranged to be tightly seated onto a top portion of a conventional replacement femoral stem 640. The implant preferably comprises a replacement femoral stem socket 642, preferably formed of polyurethane having a Shore hardness designated as 70D and having an outer facing rim 644. Preferably mounted onto a circumferential recess 646 formed on an outer surface of socket 642 is a generally ball shaped hollow outer element 648, preferably formed of polyurethane having a Shore hardness designated as 80A and defining an articulating surface 650. Disposed within element 648 is a core element 652, preferably formed of foamed polyurethane of the same type used in element 648. Located between elements 648 and 652 is at least one relatively thin reinforcing layer 654, typically formed of KEVLAR® or carbon cloth. Core element 652 may be advantageously formed with at least one fluid-filled void 656.

At least one selectably inflatable, selectably expandable void 658 is preferably provided between element 648 and reinforcing layer 654 over generally the entire areas thereof. At least one inflation passageway 660 having an associated one-way valve 662, communicates between a location adjacent rim 644 and the interior of void 658 to enable a spherical radius determining material to be selectably inserted into void 658 in order to enable adaptation of the overall radius of the generally spherical articulating surface 650 in situ so as to provide personalized fit of the ball implant to a patient's acetabulum socket.

FIG. 23C shows a further alternative embodiment of the implant 668 as a generally ball-shaped, partially hollow multi-layer assembly arranged to be tightly seated onto a top portion of a conventional replacement femoral stem 670. The implant preferably comprises a replacement femoral stem socket 672, preferably formed of polyurethane having a Shore hardness designated as 70D and having an outer facing rim 674. Preferably mounted onto a circumferential recess 676 formed on an outer surface of socket 672 is a generally ball shaped hollow outer element 678, preferably formed of polyurethane having a Shore hardness designated as 80A and defining an articulating surface 680. Disposed within element 678 is a core element 682, preferably formed of foamed polyurethane of the same type used in element 678. Located between elements 678 and 682 is at least one relatively thin reinforcing layer 684, typically formed of KEVLAR® or carbon cloth. Core element 622 may be advantageously formed with fluid-filled voids, which may be, for example, in a generally spherical shape, as shown at reference numeral 686 or a generally annular shape, as shown at reference numeral 688. The voids provide desired flexibility of core element 682.

Reference is now made to FIGS. 24A & 24B, which are simplified illustrations of aspects of the functionality of the embodiments illustrated respectively in FIGS. 23A & 23B. FIG. 24A shows at A the implant 602 in the embodiment of FIG. 23A, disposed in articulating engagement with an acetabulum socket 690 and a force, indicated by an arrow 692 being exerted on the ball implant 602 via the socket 690. Such a situation occurs cyclically when a person is walking or running. The force, indicated by arrow 692 produces deformation of the ball implant 602 and consequent temporary reshaping of the articulating surface 620, from its non-deformed geometry as shown at reference numeral 694 to a deformed geometry as shown at reference numeral 696.

The deformation of ball implant 602 produces compression of core element 622, reinforcing layer 624 and hollow outer element 618, which are shown as a single layer 622, which compression effectively reduces the volume of void 626 and thus causes flow of synovial fluid from void 626 via passageways 632 and one-way valves (not shown) into the articulation region 697, as shown by arrows 698.

At stage B there is seen the implant 602 in the embodiment of FIG. 23A, disposed in articulating engagement with an acetabulum socket 690 in the absence of the force, indicated by an arrow 692 at stage A being exerted on the ball implant 602 via the socket 690. Such a situation occurs cyclically when a person is walking or running, interspersed with the situation shown at stage A. The absence of the force, indicated by arrow 692, eliminates deformation of the ball implant 602 and allows articulating surface 620 to return to its non-deformed geometry as shown at reference numeral 694 from the deformed geometry as shown at reference numeral 696.

The elimination of the deformation of ball implant 602 eliminates compression of core element 622, reinforcing layer 624 and hollow outer element 618, which are shown as a single layer 622, which effectively returns the volume of void 626 to its volume prior to stage A, resulting in the application of suction and thus causes flow of synovial fluid to void 626 via passageways 630 and one-way valves (not shown), as shown by arrows 699.

FIG. 24B shows at A the implant 604 in the embodiment of FIG. 23B, initially placed in articulating engagement with a natural acetabulum socket 710. Preferably a relatively thin spacer element 712 is interposed between the implant 604 and the acetabulum socket 710. It is seen that the outer radius of the articulating surface 650 of implant 604 is less than a desired radius for articulation with a corresponding articulating surface 714 of acetabulum socket 710.

At stage B, insertion of a fluid, preferably but not necessarily a settable polyurethane material, for example in situ curable polyurethane which is commercially available from Advanced Bio Surfaces, Inc. of 5909 Baker Road, Suite 550, Minnetonka, Minn., via at least one passageway 660 and corresponding one-way valve 662 into void 658, thereby at least partially filling the void and producing expansion of element 648 preferably in a spherically uniform manner, thereby increasing the radius of the articulating surface 650.

At stage C, insertion of a fluid, preferably but not necessarily a settable polyurethane material, for example in situ curable polyurethane which is commercially available from Advanced Bio Surfaces, Inc. of 5909 Baker Road, Suite 550, Minnetonka, Minn., via at least one passageway 660 and corresponding one-way valve 662 into void 658, thereby bring the radius of the articulating surface 650 to a desired radius, which is smaller than the radius of the articulating surface 714 of the acetabulum socket 710 by an amount represented by the thickness of the spacer element 712.

At stage D, the spacer element 712 is removed, thus allowing a desired clearance 718 between the respective articulating surfaces 650 and 714 of the ball implant 604 and the acetabulum socket 710, to be filled by synovial fluid 720.

It is appreciated that in this manner, regeneration of cartilage along the articulating surface 714 of the ball implant 604 may be enhanced by the precise matching of radii of surfaces 650 and 714.

It is appreciated that the ball implant devices shown in FIGS. 22-24B may be configured and constructed as unitary devices, including a stem, rather than being mounted on a stem via a sleeve.

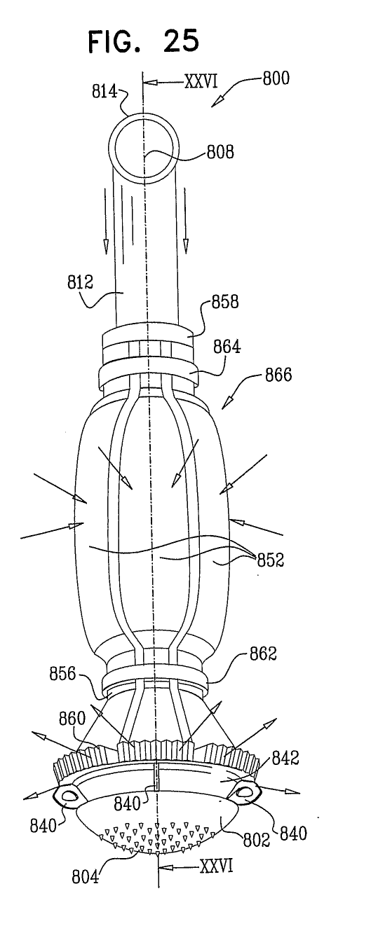

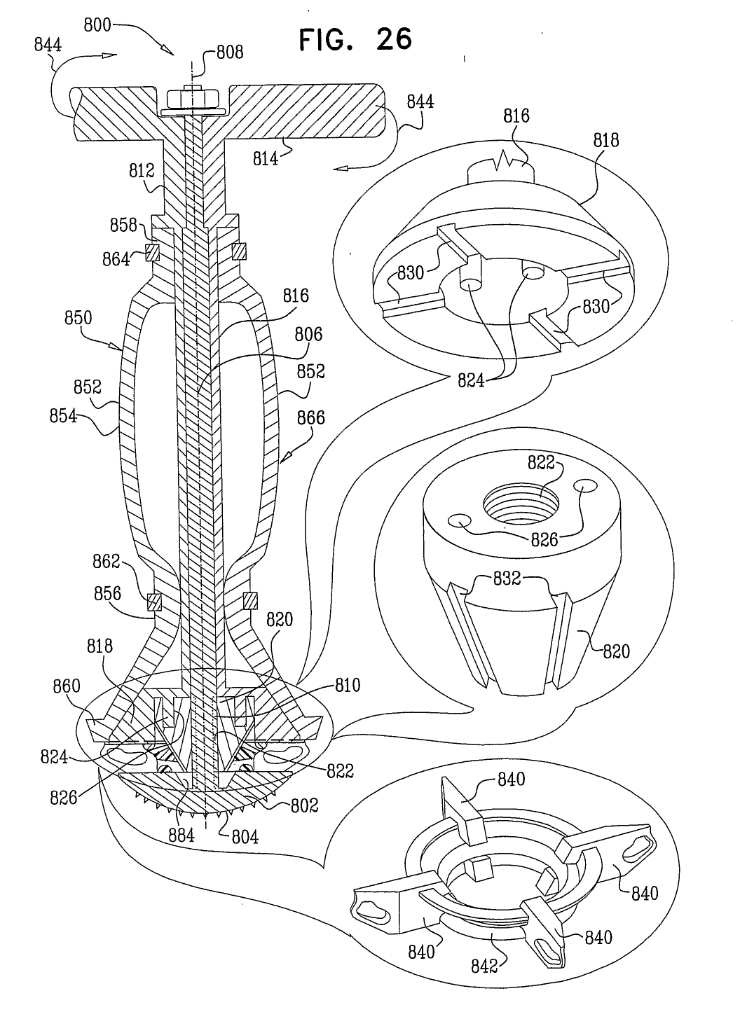

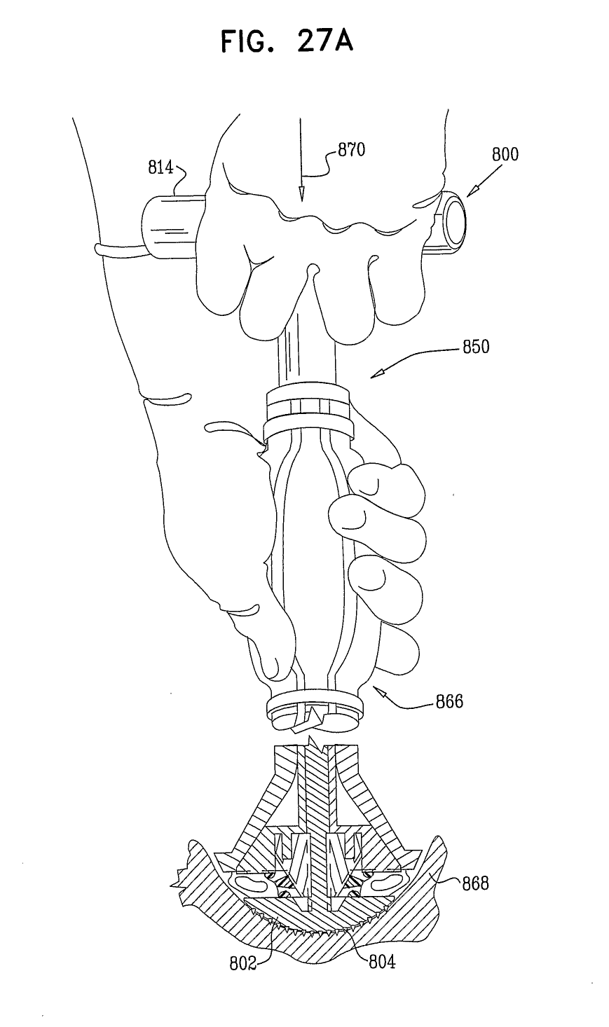

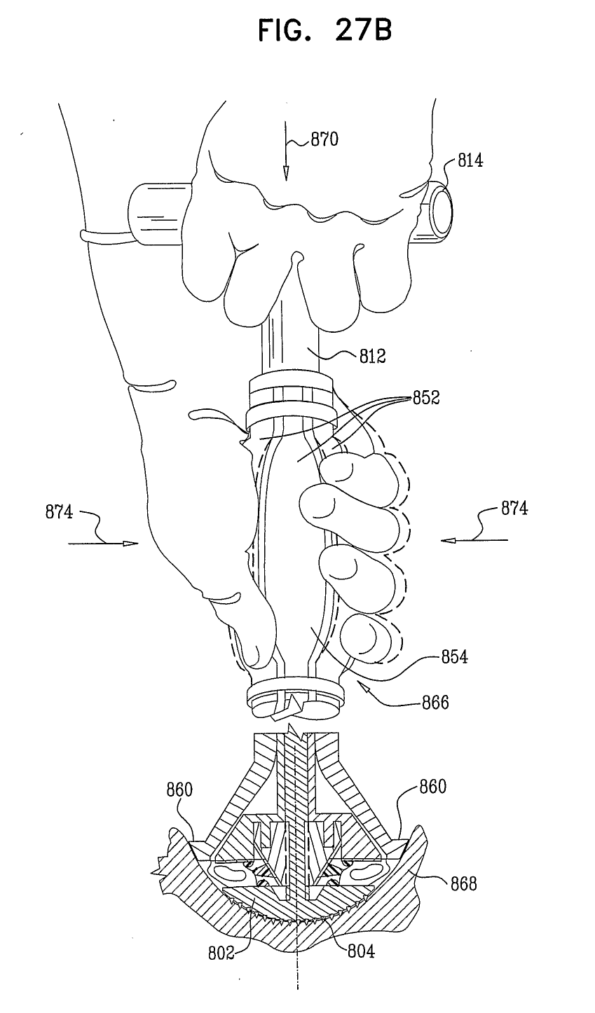

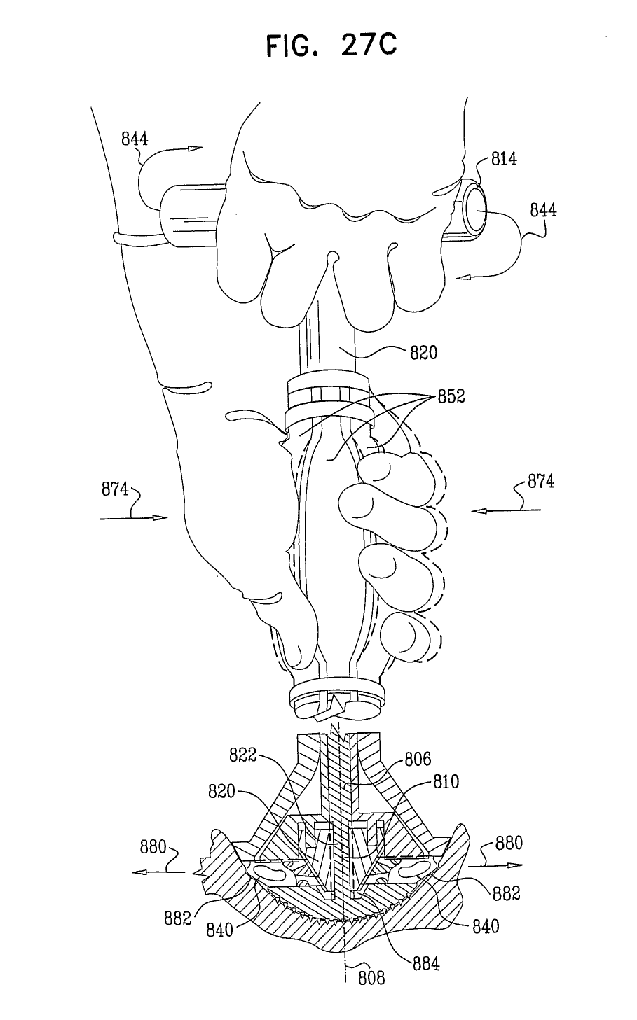

Reference is now made to FIG. 25, which is a simplified pictorial illustration of a groove reamer constructed and operative in accordance with a preferred embodiment of the present invention, useful, for example in the embodiment of FIG. 13, to FIG. 26, which is a simplified composite illustration showing the structure of the groove reamer of FIG. 25 and to FIGS. 27A, 27B, 27C and 27D, which are simplified sectional illustrations illustrating various stages of the operation of the groove reamer of FIGS. 25 and 26.

As seen in FIGS. 25 and 26, there is provided a groove reamer 800 comprising a central bone anchor element 802 which is preferably integrally formed of a generally spherical spiked bone engagement surface 804 and a central shaft 806 extending along an axis 808, having a threaded portion 810 adjacent the bottom thereof. The central bone anchor element 802 is preferably formed of metal, such as titanium or aluminum.

Mounted for rotation about central shaft 806 is a rotational driving assembly 812 including a first handle 814, preferably integrally formed with an elongate hollow shaft 816, which is sized to rotationally accommodate central shaft 806. Hollow shaft 816 terminates in a rotational driving plate 818. Coupled for rotation together with rotational driving plate 818 is a conical rotational and axial driving element 820 having a central threaded bore 822, which threadably engages threaded portion 810 of central shaft 806. Preferably rotational driving plate 818 is formed with a plurality of axially extending pins 824 which extend into corresponding axially extending sockets 826 formed in conical rotational and axial driving element 820.

In accordance with a preferred embodiment of the present invention, rotational driving plate 818 is formed with a plurality of slidable knife support channels 830 and conical rotational and axial driving element 820 is formed with a plurality of correspondingly positioned slidable knife support channels 832, which extend generally at an angle with respect to channels 830.

A plurality of knives 840 are each slidably seated in a pair of corresponding channels 830 and 832 and are mounted on a resilient knife support ring 842 which permits simultaneous radially outward and rotational displacement of the knives 840 in response to simultaneous axial and rotational movement of conical rotational and axial driving element 820 in threaded engagement with threaded portion 810 of central shaft 806 in response to rotation of first handle 814 in a direction indicated by an arrow 844.

A radially displaceable bone engagement assembly 850, typically comprises a plurality of integrally formed flexible engagement elements 852, each comprising a hand engageable portion 854, lying intermediate first and second retaining portions 856 and 858 and a radial bone engaging tooth portion 860. Assembly 850 preferably comprises six integrally formed flexible engagement elements 852 which are held together about hollow shaft 816 at respective first and second retaining portions 856 and 858 by a corresponding pair of retaining bands 862 and 864 to collectively define a second handle 866 at hand engageable portions 854 thereof.

As will be described hereinbelow in greater detail, an operator, such as a surgeon, grasping second handle 866 with one hand causes bending of flexible engagement elements 852 about retaining portions 856, causing bone engaging tooth portions 860 to be displaced radially outwardly into retaining engagement with the walls of a bone socket being reamed.

Referring now to FIGS. 27A-27D, it is seen that initially, as seen in FIG. 27A, the surgeon places the reamer 800 with central bone anchor element 802 in a bone socket 868 to be reamed and pushes first handle 814 axially downwardly as indicated by an arrow 870, causing the spikes on generally spherical spiked bone engagement surface 804 to engage the bone surface at socket 868. This anchors the central bone anchor element 802 against rotation with respect to socket 868.

FIG. 27B shows engagement of the radial bone engagement tooth portions 860 with side surfaces of the socket 868. This engagement is produced by the surgeon squeezing his hand which engages the second handle 866 and thus forcing the hand engageable portions 854 of integrally formed flexible engagement elements 852 radially inwardly, as indicated by arrows 874, producing corresponding radially outward displacement of tooth portions 860 into retaining engagement with the side surfaces of socket 868. This further stabilizes the reamer with respect to the bone socket 868.

FIG. 27C illustrates the beginning of reaming operation, which is produced by rotation of first handle 814 about axis 808 such as in a direction indicated by arrows 844. The rotation of first handle 814 causes rotation of conical rotational and axial driving element 820 about axis 808 and consequent corresponding axially forward displacement of conical rotational and axial driving element 820 due to the threaded engagement of threaded portion 810 of central shaft 806 with threaded bore 822. This forward movement of conical rotational and axial driving element 820 drives knives 840, which are slidably seated in channels 830 and 832 (FIG. 26) in a radially outward direction, indicated by arrows 880, into cutting engagement with the bone socket, thus beginning to produce a circumferential channel 882 therein. As seen in FIG. 27C, a recess 884 is preferably formed in the central bone anchor element 802.

FIG. 27D illustrates the completion of the reaming operation produced by rotation of first handle 814 about axis 808 such as in a direction indicated by arrows 844. Continued rotation of first handle 814 causes rotation of conical rotational and axial driving element 820 about axis 808 and consequent corresponding axially forward displacement of conical rotational and axial driving element 820 due to the threaded engagement of threaded portion 810 of central shaft 806 with threaded bore 822. This forward movement of conical rotational and axial driving element 820 drives knives 840, slidably seated in channels 830 and 832 (FIG. 26) further in a radially outward direction, indicated by arrows 880, into cutting engagement with the bone socket, thus beginning to produce a circumferential channel 882 therein. The rotational and axial displacement of the conical rotational and axial driving element 820 typically stops when the conical rotational and axial driving element 820 engages the bottom of a corresponding recess 884 formed in the central bone anchor element 802 above the generally spherical spiked bone engagement surface 804.

It is a particular feature of the present invention that the knives 840 are slidably supported for radial displacement and cutting by the mechanism described hereinabove.

Removal of the reamer 800 may readily be accomplished by rotating the first handle in an opposite direction. The resiliency of ring 842 is operative to radially retract the knives 840.

It is appreciated that the extension and retraction of knives 840, may be gauged by a gauging apparatus, and the gauging may be displayed to an operator for monitoring the displacement of knives 840.

It is also appreciated that the rotation of elongate hollow shaft 816 and rotational driving plate 818 may be empowered by an electronic or hydraulic system, and the operator may utilize the display of extension and retraction of the knives to determine the completion of groove 882 to a precise desired depth. The electronic or hydraulic system may replace handle 814.

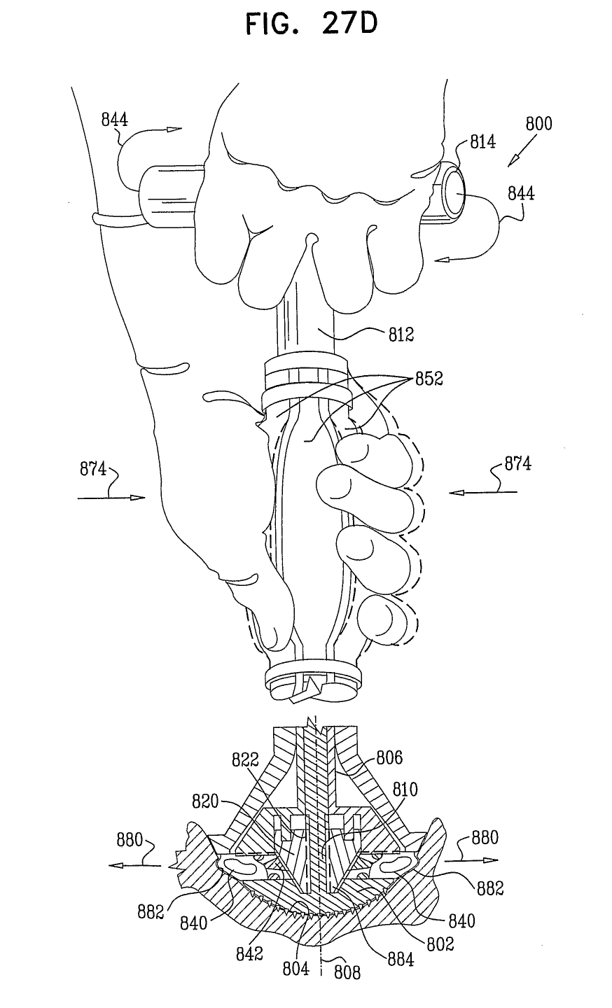

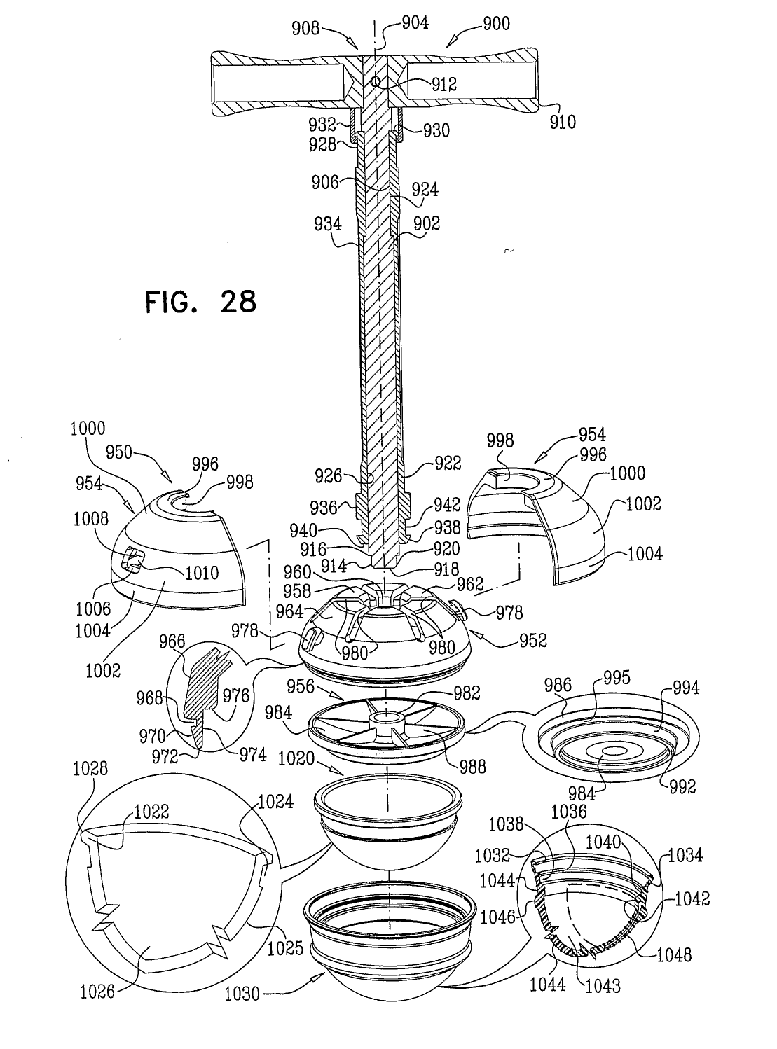

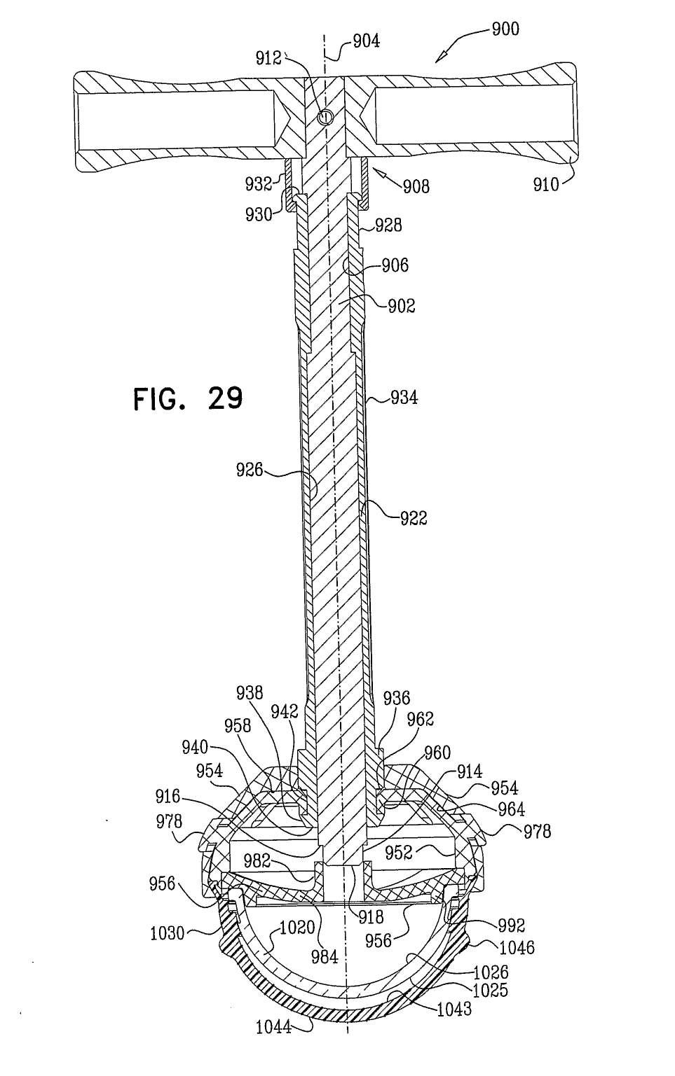

Reference is now FIG. 28, which is a simplified composite partially sectional exploded-view illustration of a socket implant assembly and implanter useful therewith, constructed and operative in accordance with a preferred embodiment of the present invention and to FIG. 29, which is a simplified composite sectional assembled-view illustration of the socket implant assembly and implanter of FIG. 28, taken in the section plane of FIG. 28. References to top, bottom, upper and lower are in the context of FIGS. 28 and 29.

As seen in FIGS. 28 and 29, there is provided an implanter 900 comprising a central shaft 902 extending along an axis 904 and having an outer facing threaded portion 906 adjacent the top thereof. Mounted onto central shaft 902 for driving rotation thereof is a rotational driving assembly 908 including handle 910, preferably fixed to central shaft 902 by a transversely extending pin 912. The bottom of central shaft 902 is formed with an end portion 914 having a reduced radius and defining a shoulder 916 with respect to the remainder of the central shaft lying thereabove. Preferably, the bottom surface 918 of the end portion 914 is formed with a bevel 920.

A sleeve 922 is disposed about central shaft 902 and includes an inner facing threaded portion 924 at the top thereof. Sleeve 922 has a generally uniform inner surface 926 extending therealong below threaded portion 924. The outer surface of sleeve 922 includes a circumferential recess 928 adjacent the top thereof, defining a retaining circumferential protrusion 930 which may be engaged by a protective sleeve 932, depending from handle 910, which preferably is provided in order to prevent inadvertent engagement of a user's hand or finger between the top of sleeve 922 and handle 910.

A central portion 934 of the outer surface of sleeve 922 is preferably knurled in order to provide a gripping surface for user engagement during implanting. Below central portion 934, there is preferably formed a peripheral protrusion 936 of uniform radius. Below peripheral protrusion 936 and spaced therefrom along axis 904 is a beveled protrusion 938, which tapers inwardly and downwardly to a bottom edge 940 of the sleeve 922. The space between peripheral protrusion 936 and beveled protrusion 938 defines a recess 942.

Arranged for operational engagement with central shaft 902 and sleeve 922 is a disposable implant mounting assembly 950 including a flexible central element 952, a pair of outer elements 954 and a bottom element 956.

Flexible central element 952 preferably is formed of a relatively rigid plastic material, such as polyethylene and is a generally rotationally symmetric. Element 952 is formed with a generally flat top surface 958 which surrounds a generally axial collar 960 and is joined thereto by a beveled edge 962. Extending outwardly and downwardly from top surface 958 is an upper peripheral outer surface 964. Extending downwardly from upper peripheral outer surface 964 is an intermediate peripheral outer surface 966 which terminates in a peripheral recess 968. Below peripheral recess is a lower peripheral surface 970 which terminates in a lower edge 972.

Interiorly of intermediate peripheral outer surface 966, peripheral recess 968 and lower peripheral surface 970 there is provided an interior facing recess 974 which defines an upper interior facing shoulder 976.

A plurality, preferably two, radially outward extending tabs 978 are formed, preferably in oppositely facing locations at the junction of upper peripheral outer surface 964 and intermediate peripheral outer surface 966.

A plurality of slits 980, preferably six in number, are preferably formed in flexible central element 952, to provide flexibility thereof, and extend radially in evenly spaced azimuthal distribution through flat top surface 958 and upper peripheral outer surface 964.

Bottom element 956 preferably is formed with a central collar portion 982 which is integrally joined at its bottom to a curved generally radially extending portion 984 having a peripheral edge 986 which is configured to be seated in recess 974 of flexible central portion 952 against shoulder 976. Preferably a plurality of radially extending ribs 988 are provided at the top of radially extending portion 984. A ring 992 integrally depends from radially extending portion 984 slightly inwardly of peripheral edge 986. Ring 992 defines a peripheral outwardly facing surface 994 and an adjacent downwardly facing surface 995.

Outer elements 954 together define a generally hemispherical body and preferably each include a thickened generally flat top facing surface 996, which defines a collar 998, an upper peripheral surface 1000, an intermediate peripheral surface 1002 and a lower peripheral surface 1004. Apertures 1006 are preferably formed in intermediate peripheral surface 1002 to accommodate tabs 978 of flexible central element 952 for desired mounting of outer elements 954 with respect thereto. Apertures 1006 each preferably include a relatively widened portion 1008 for receiving a tab 978 and a relatively narrowed portion 1010 adjacent widened portion 1008 for retaining tab 978.

An inner, relatively rigid implant element 1020, preferably formed of metal or ceramic, preferably includes a generally circular peripheral rim 1022 below which is defined a generally circular, peripheral, outer-facing recess 1024. The surface portion 1025 of the outer surface of element 1020 is generally spherical. The interior surface 1026 of element 1020 defines a generally spherical articulating surface. Rim 1022 is preferably formed with a beveled edge 1028.

An outer, relatively resilient implant element 1030, preferably formed of polyurethane, preferably includes a generally circular inwardly facing peripheral rim 1032, which is adapted to be seated in recess 968 of flexible central element 952. Below rim 1032 is an inwardly tapered portion 1034 which terminates in an inwardly facing peripheral protrusion 1036 having a downwardly facing beveled edge 1038 adapted to engage beveled edge 1028 of inner implant element 1020.

Below beveled edge 1038 is an inwardly facing recess 1040 which is adapted to engage rim 1022 of inner implant element 1020. Disposed below inwardly facing recess 1040 is a inwardly facing protrusion 1042 which is adapted to engage recess 1024 of inner implant element 1020. The portion 1043 of the inner facing surface of outer implant element 1030 lying below protrusion 1042 is generally spherical and adapted to engage surface portion 1025 of inner implant element 1020.

The outer surface 1044 of implant element 1030 is generally spherical and preferably includes a peripheral protrusion 1046 which lies intermediate therealong.

In accordance with a preferred embodiment of the present invention, reinforcing material 1048 may be provided at one or more locations between surface portions 1043 and 1044. Preferably such reinforcement is provided generally at a location which lies against a naturally occurring acetabulum notch in a patient's acetabulum.

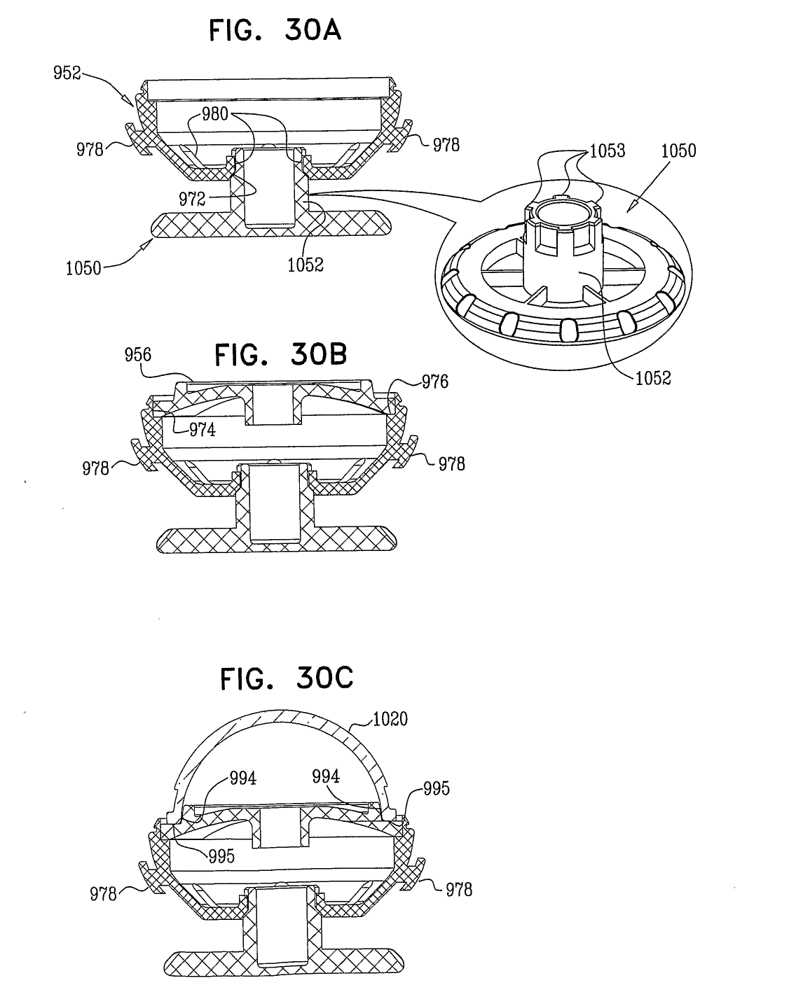

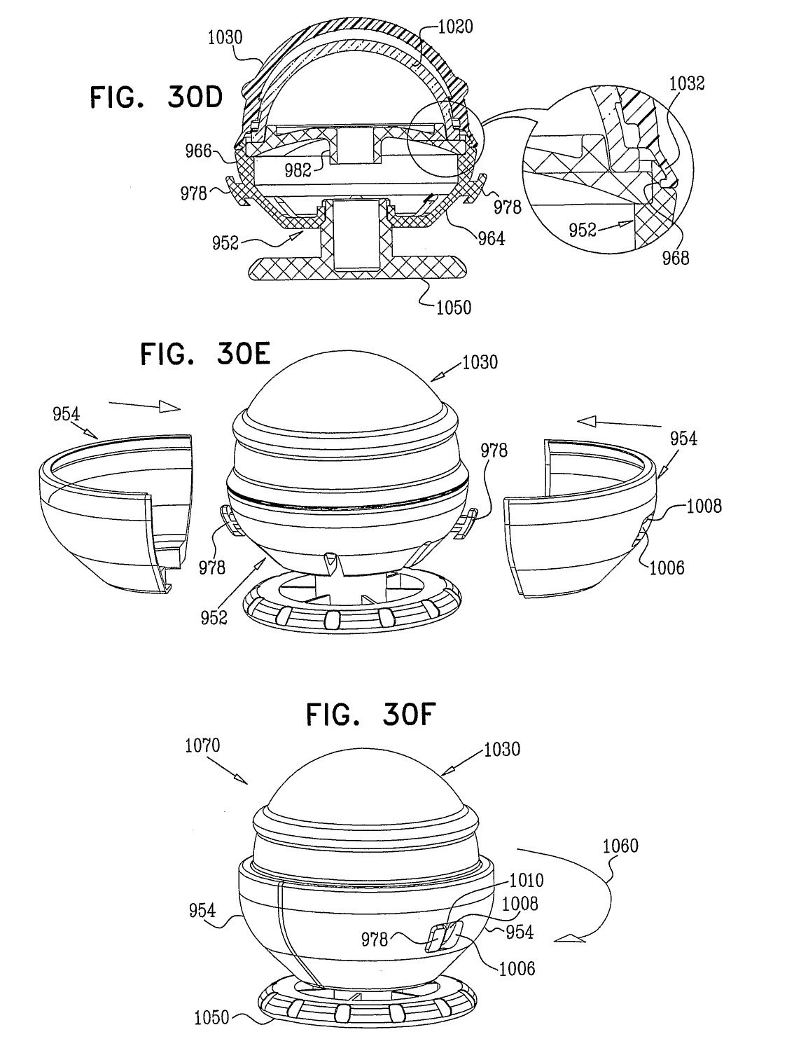

Reference is now made to FIGS. 30A, 30B, 30C, 30D, 30E and 30F, which are simplified illustrations of assembly of the socket implant assembly of FIGS. 28 and 29. FIG. 30A, shows the flexible central element 952 mounted upside-down on an assembly fixture 1050 having an elongate portion 1052 including at least two elongate protrusions 1053 which are adapted to be seated in at least one pair of opposite slits 980, thereby preventing relative rotation between the flexible central element 952 and the assembly fixture 1050.

FIG. 30B shows bottom element 956 seated in recess 974 against shoulder 976. FIG. 30C shows inner implant element 1020 seated at the junction of surfaces 994 and 995 of bottom element 956.

FIG. 30D shows outer implant element 1030 seated with rim 1032 in recess 968 of flexible central element 952. FIG. 30E shows outer elements 954 being displaced axially inwardly with respect to flexible central element 952 such that tabs 978 engage relatively wide portions 1008 of apertures 1006.

FIG. 30F shows outer elements 954 being rotated in a direction indicated by an arrow 1060 relative to mounting appliance 1050 and to flexible central element 952, such that tabs 978 engage relatively narrow portions 1010 of apertures 1006, thus retaining outer elements 954 in desired positions relative to flexible central element 952. Thereafter, the assembled socket implant assembly 1070, including the disposable implant mounting assembly 950 together with implant elements 1020 and 1030, may be detached from mounting appliance 1050.

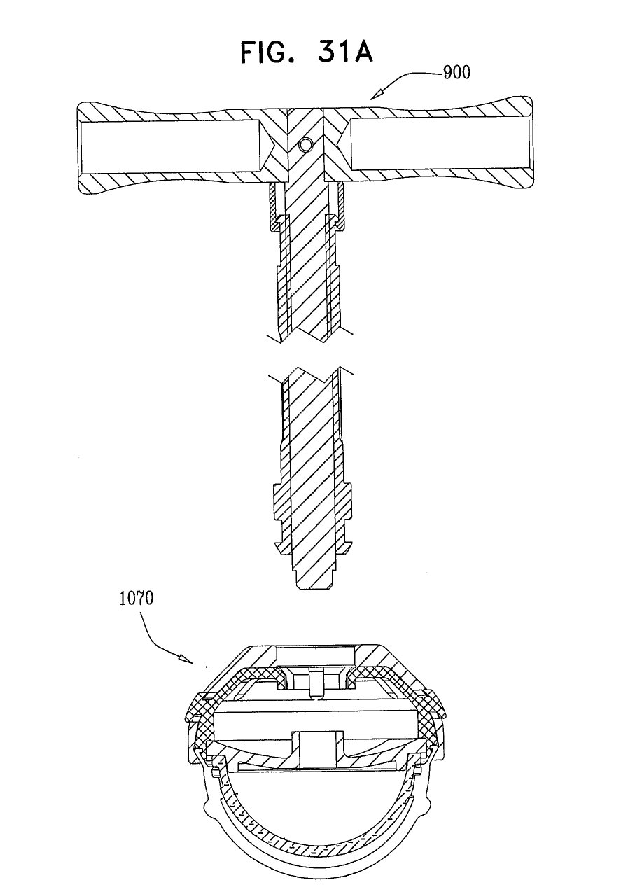

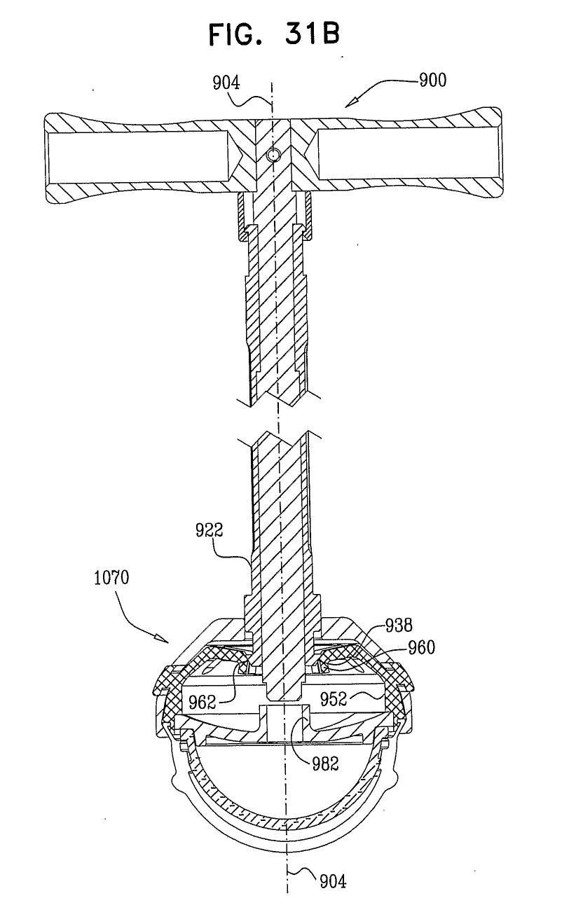

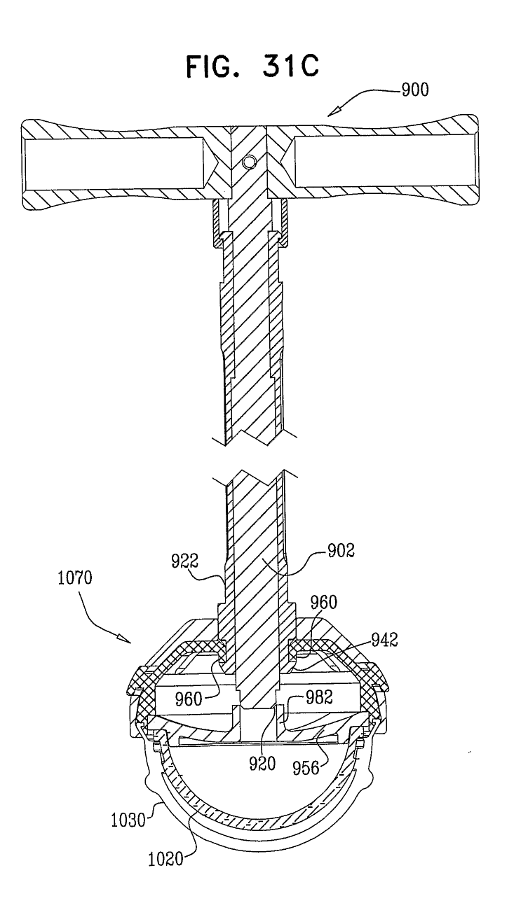

Reference is now made to FIGS. 31A, 31B and 31C are simplified illustrations of mounting of a socket implant assembly 1070 onto an implanter tool, a function which is preferably performed at a hospital by a nurse or technician.

FIG. 31A shows the assembled socket implant assembly 1070 ready for mounting onto the implanter 900. FIG. 31B shows a first stage of relative axial displacement of the assembled socket implant assembly 1070 and the implanter 900 along axis 904. It is seen that beveled edge 938 of sleeve 922 of the implanter 900 engages beveled edge 962 of flexible central element 952 and forces collar 960 outwardly, preliminary to snap-fit engagement therewith. FIG. 31C shows snap-fit engagement of collar 960 in recess 942 of sleeve 922. It is noted that the beveled edge 920 of central shaft 902 is located at the top of central collar portion 982 of bottom element 956. The apparatus of FIGS. 28 and 29 is now ready for insertion of the implant elements 1020 and 1030 into the acetabulum of a patient.

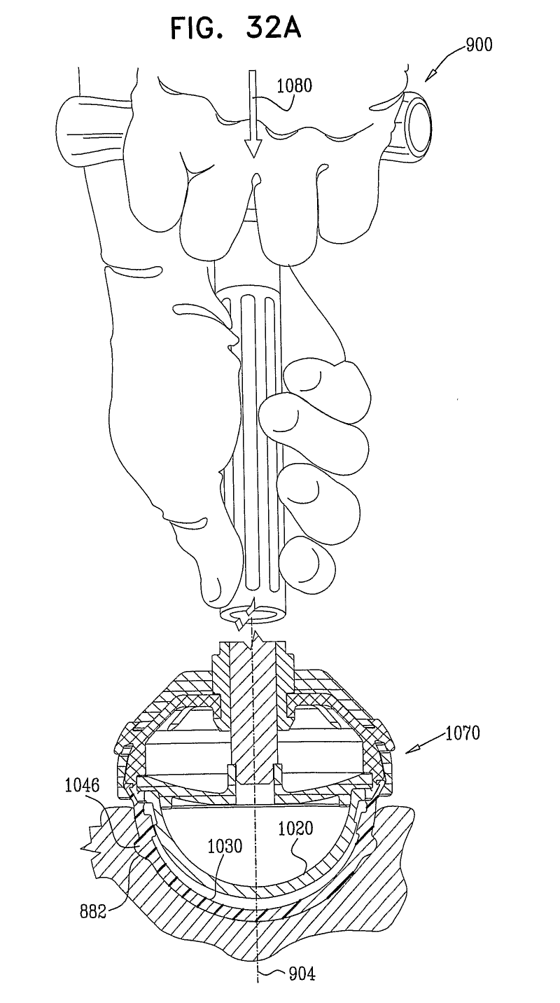

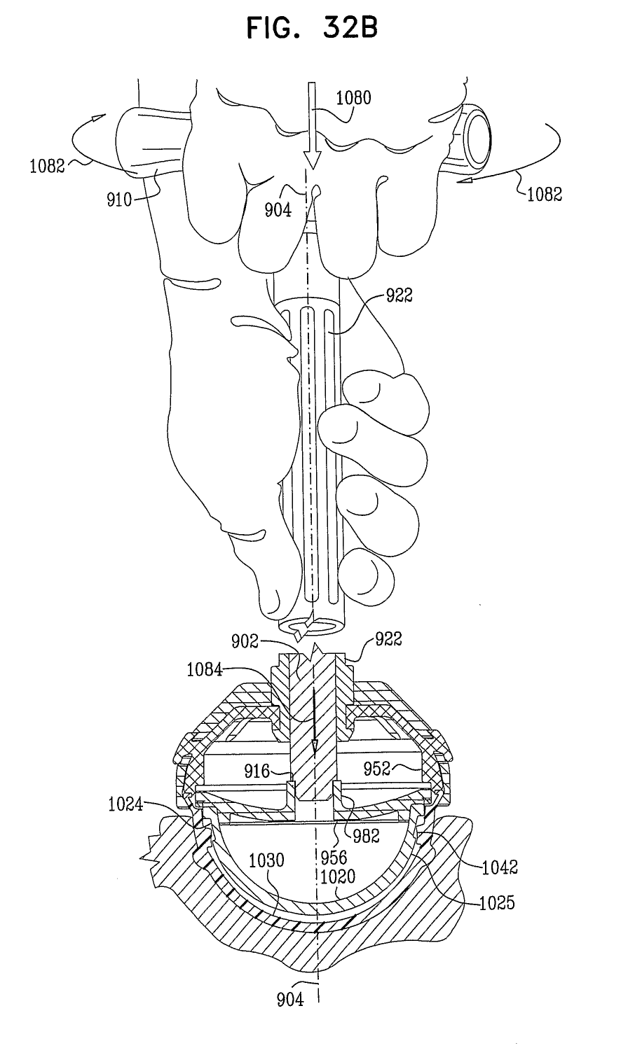

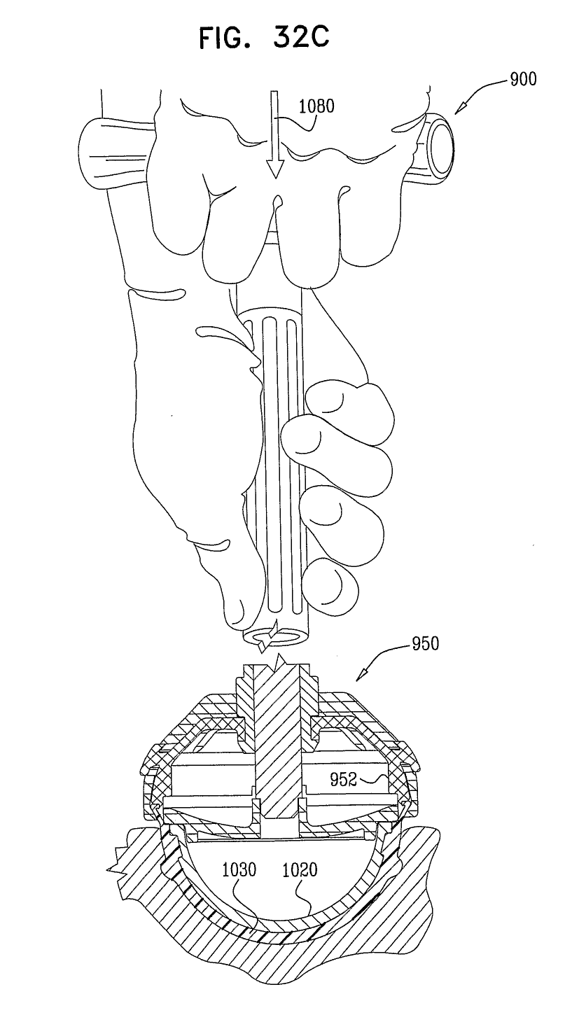

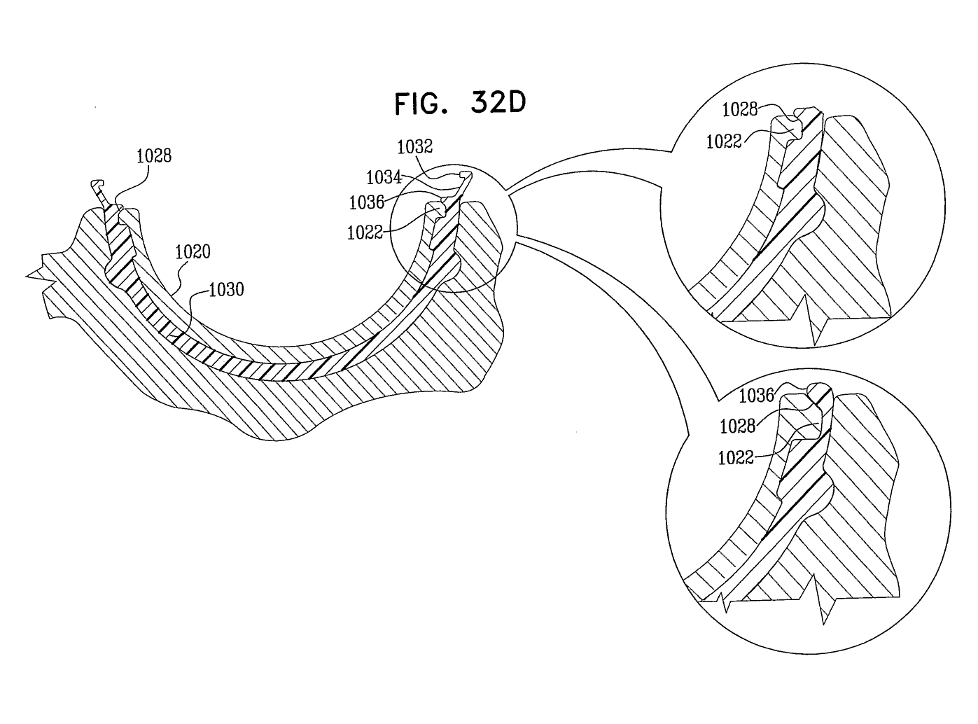

Reference is now made to FIGS. 32A, 32B, 32C and 32D, which are simplified illustrations of implanting a socket implant employing the apparatus of FIGS. 28 and 29. Prior to initial insertion of the assembled socket implant assembly 1070, the patient's acetabulum is preferably spherically reamed in a conventional manner and is then reamed to define circumferential groove 882 (FIG. 27D), preferably in a manner described hereinabove with reference to FIGS. 25-27D.

FIG. 32A shows snap fit engagement of protrusion 1046 of outer implant element 1030 with circumferential groove 882. This snap fit engagement is produced by axial motion of the implanter 900 along axis 904 in a direction indicated by an arrow 1080.

FIG. 32B shows rotation of the handle 910 about axis 904 in a direction indicated by an arrow 1082, while sleeve 922 is held static. The rotation of handle 910 produces downward axial displacement of central shaft 902, in a direction indicated by an arrow 1084 relative to sleeve 922 due to the threaded engagement therebetween and thus relative to the flexible central element 952. This axial displacement causes downward displacement of bottom element 956 and inner implant element 1020 relative to flexible central element 952 in the direction indicated by arrow 1084 due to engagement of shoulder 916 of central shaft 902 with central collar portion 982 of bottom element 956. Engagement of outer surface 1025 of inner implant element 1020 with protrusion 1042 of outer implant element 1030 produces slight deformation of protrusion 1042, preliminary to snap-fit engagement between inner implant element 1020 and outer implant element 1030 due to engagement between protrusion 1042 and recess 1024 formed on the outer surface of the inner implant element 1020.

FIG. 32C shows the full snap fit engagement. FIG. 32D shows the inner and outer implant elements 1020 and 1030 in place in the acetabulum of the patient, decoupled from the implanter 900 and the disposable implant mounting assembly 950. It is seen that portions 1032 and 1034 of the outer implant element are preferably removed. Alternatively, portions 1032 and 1034 may be obviated in an arrangement where protrusion 1036 engages a corresponding recess on the central flexible element 952 (FIG. 28). It is a particular feature of an embodiment of the present invention that protrusion 1036 of the outer implant element covers and thus protects edge 1028 of rim 1022 of inner implant element.

It is appreciated that an outer implantable element, such as outer implantable element 1030, may be formed with a plurality of discrete protrusions, adapted to engage corresponding discrete recesses in the acetabulum of the patient. In such an embodiment, an inner implantable element, such as inner implantable element 1020, may be formed with a plurality of discrete protrusions adapted to engage the outer implantable element.

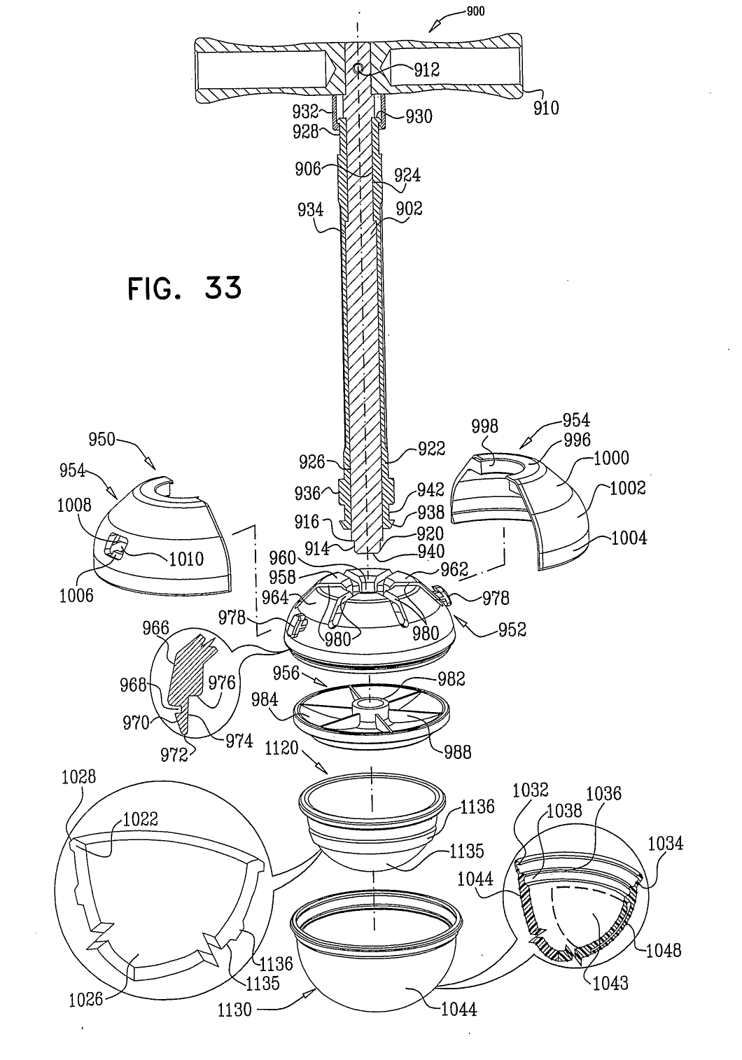

Reference is now made to FIG. 33, which is a simplified composite exploded-view illustration of a socket implant assembly and implanter useful therewith, constructed and operative in accordance with another preferred embodiment of the present invention. The embodiment of FIG. 33 is identical to that of FIGS. 28 and 29 with a variation in the inner implant element, here designated by reference numeral 1120 and the outer implant element, here designated by reference numeral 1130.

In this embodiment an outer surface 1135 of the inner implant element 1120 is smooth other than for a circumferential protrusion 1136, intermediate therealong. The outer implant element 1130 is formed without protrusions 1042 and 1046 which were present in the embodiment of FIGS. 28-32D.

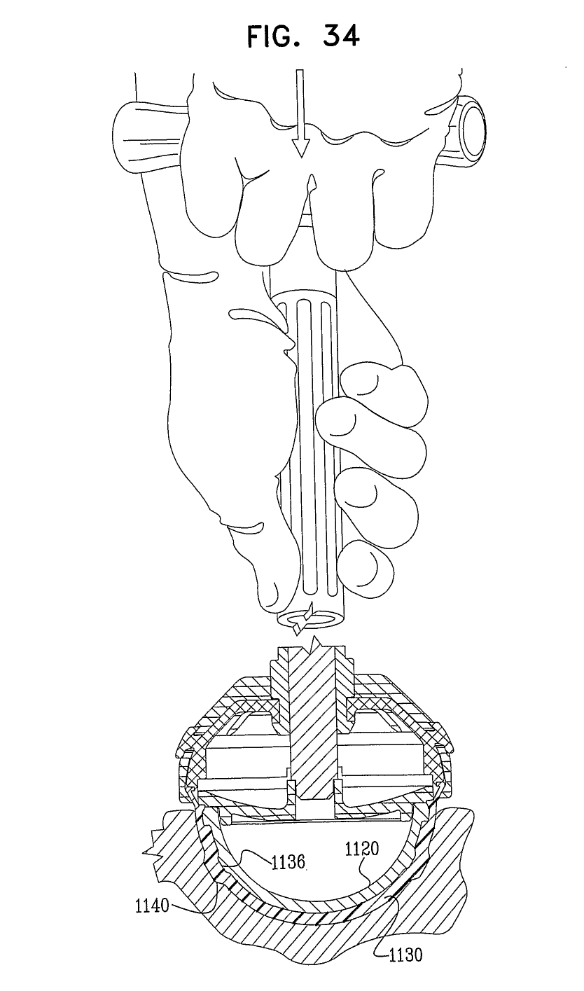

FIG. 34 shows full insertion of inner and outer implant elements 1120 and 1130. It is seen that protrusion 1136 forces a correspondingly located portion of outer implant element 1130 into locking engagement with a corresponding reamed recess 1140 formed in the patient's acetabulum.

It is appreciated that the gripping mechanism of implanter 900 and disposable implant mounting assembly 950 may alternatively be constructed as an integral mechanism, such as a gripping claws mechanism, of an implanter such as implanter 900.

It is appreciated that an outer implantable element, such as outer implantable element 1130, may be adapted to engage discrete recesses in the acetabulum of the patient. In such an embodiment, an inner implantable element, such as inner implantable element 1120, may be formed with a plurality of discrete protrusions adapted to engage the outer implantable element.

It will be appreciated by persons skilled in the art that the present invention is not limited by what has been particularly shown and described hereinabove. Rather the invention includes both combinations and sub-combinations of various features described herein as well as variations which would occur to persons reading the foregoing description and which are not in the prior art.

1. An orthopedic implant comprising: an implant element adapted to be attached to at least one bone, said implant element including at least one throughgoing bone growth region adapted to be located in bone growth communication with said at least one bone and configured such that bone growth therein creates a grown bone suture binding said implant element to said at least one bone.

2. An orthopedic implant comprising: a resilient implant element adapted to be attached to at least one bone, said implant element including at least one bone growth region adapted to be located in bone growth communication with said at least one bone and configured such that bone growth therein creates a grown bone lock binding said resilient implant element to said at least one bone.

3. An orthopedic implant comprising: an implant element adapted to be attached to at least one bone, said implant element including at least one bone growth region adapted to be located in bone growth communication with said at least one bone and containing grown bone enhancing material configured such that bone growth therein creates a grown bone lock binding said implant element to said at least one bone.

4-30. (canceled)

31. An orthopedic implant comprising: a ligament attachment assembly adapted to be attached to at least one bone and at least one ligament, said ligament attachment assembly including at least one ligament attachment element having at least one throughgoing bone growth region adapted to be located in bone growth communication with said at least one bone and configured such that bone growth therein creates a grown bone suture binding said at least one ligament attachment element to said at least one bone.

32. An orthopedic implant comprising: a ligament attachment assembly adapted to be attached to at least one bone, said ligament attachment assembly including at least one ligament attachment element having at least one bone growth region adapted to be located in bone growth communication with said at least one bone and configured such that bone growth therein creates a grown bone lock binding said at least one ligament attachment element to said at least one bone.

33. An orthopedic implant comprising: a ligament attachment assembly adapted to be attached to at least one bone, said ligament attachment assembly including at least one ligament attachment element having at least one bone growth region adapted to be located in bone growth communication with said at least one bone and containing grown bone enhancing material configured such that bone growth therein creates a grown bone lock binding said at least one ligament attachment element to said at least one bone.

34-51. (canceled)

52. An orthopedic implant comprising: a conformal, resilient element adapted to be inserted between respective articulating joint surfaces of a patient, said conformal resilient element including at least one position maintaining engagement portion for attachment to patient tissue in the vicinity of said articulating joint surfaces.

53-75. (canceled)

76. An orthopedic implant comprising: a resilient, at least partially hollow, meniscus implant element adapted to be inserted between respective articulating knee joint surfaces of a knee joint of a patient, said resilient meniscus implant element including at least one position maintaining engagement portion for attachment to patient tissue in the vicinity of said articulating knee joint surfaces.

77-107. (canceled)

108. An orthopedic implant comprising: a shock-absorbing ball assembly including a stem mounting portion and a multi-layer shock-absorbing, ball-defining portion defining a generally ball-shaped articulating surface.

109-124. (canceled)

125. A surgical groove reamer comprising: a central bone anchor element including a generally spherical bone engagement surface and a central shaft extending along a longitudinal axis, said central shaft having a first threaded portion thereon; a rotational driving assembly, mounted for rotation about said central shaft and including a first handle fixedly associated with an elongate hollow shaft, which is sized to rotationally accommodate said central shaft, said hollow shaft terminating in a rotational driving plate; a rotational and axial driving element, coupled for rotation together with said rotational driving plate, said rotational and axial driving element including a second threaded portion, which threadably engages said first threaded portion.

126-131. (canceled)

132. A socket implanting assembly comprising: a socket implant assembly including inner and outer implantable socket elements and a socket element mounting assembly adapted for preassembly with said inner and outer implantable socket elements; and an implanter tool operative with said socket implant assembly for implanting said implantable socket elements in a reamed socket in a patient's joint.

133. A socket implanting assembly comprising an implanter tool useful with a socket implant assembly including inner and outer implantable socket elements and a socket element mounting assembly adapted for preassembly with said inner and outer implantable socket elements for implanting said implantable socket elements in a reamed socket in a patient's joint.

134. A socket implanting assembly for use with an implanter tool and comprising: a socket implant assembly including inner and outer implantable socket elements and a socket element mounting assembly adapted for preassembly with said inner and outer implantable socket elements.

135. A socket implanting assembly for use with an implanter tool and inner and outer implantable socket elements, said socket implanting assembly comprising: a socket implant mounting assembly adapted for preassembly with said inner and outer implantable socket elements and for implanting operation in cooperation with said implanter tool.

136-162. (canceled)

163. A method of alleviating difficulties in joint articulation of a patient comprising: placing a conformal, resilient element between respective articulating joint surfaces of said patient; and retaining said conformal resilient element in a desired position with respect to said respective articulating joint surfaces by attachment thereof to patient tissue in the vicinity of said articulating joint surfaces.

164. A method of implanting a bone implant element including a bone growth region, said method comprising the steps of: positioning said bone implant element with said bone growth region in contact with a patient's bone surface; encouraging bone growth from said bone surface into said bone growth region; and completing bone growth throughout said bone growth region, thereby locking said bone implant element to said bone surface within a few months.

165. A method of implanting a bone implant element including at least one bone growth region and at least one drug delivery channel communicating therewith, said method comprising the steps of: positioning said bone implant element with said at least one bone growth region in contact with a patient's bone surface; encouraging bone growth from said bone surface into said at least one bone growth region; effecting drug delivery through said at least one drug delivery channel to said bone surface; and completing bone growth throughout said at least one bone growth region, thereby locking said bone implant element to said bone surface.

166. A method of implanting a bone splint implant element including at least two bone growth regions, said method comprising the steps of: positioning said bone splint implant element with at least one of said at least two bone growth regions in contact with bone surfaces of each of at least two bone elements; encouraging bone growth from each of said bone surfaces into said at least two bone growth regions; and completing bone growth throughout said at least two bone growth regions, thereby locking said bone splint implant element to said bone surfaces.

167. A method of implanting a ligament attachment assembly including at least one bone growth region, said method comprising the steps of: forming a hole in a bone in a manner providing a circumferential undercut, having a first circumferential surface and therebelow a second circumferential surface which are joined to define said circumferential undercut; providing said ligament attachment assembly, including a plurality of ligament attachment elements, each having looped therearound, at a ligament loop retaining location thereof, a closed looped ligament end; and inserting said ligament attachment assembly into said hole and locating said plurality of ligament attachment elements in bone growth communication with a bone surface defined by said hole and configured such that bone growth thereat creates a grown bone ligament anchor binding said plurality of ligament attachment elements to said bone.

168-169. (canceled)

170. A method of implanting an inter-surface articulating joint implant, said method comprising the steps of: surgically inserting said inter-surface articulating joint implant between an acetabulum socket and a femoral head when said inter-surface articulating joint implant does not necessarily conform to corresponding shapes of articulating surfaces of said acetabulum socket and said femoral head; by means of at least some articulation of the hip joint, causing the shape of said inter-surface articulating joint implant to become increasingly conformal to said corresponding shapes of said articulating surfaces of said acetabulum socket and said femoral head; and causing said inter-surface articulating joint implant to float in synovial fluid between said articulating surfaces of said acetabulum socket and said femoral head.

171. A method of implanting an inter-surface articulating joint implant, said method comprising the steps of: surgically inserting said inter-surface articulating joint implant between an acetabulum socket and a femoral head when said inter-surface articulating joint implant does not necessarily conform to corresponding shapes of articulating surfaces of said acetabulum socket and said femoral head; by means of at least some articulation of the hip joint, causing the shape of said inter-surface articulating joint implant to become increasingly conformal to said corresponding shapes of said articulating surfaces of said acetabulum socket and said femoral head; and causing said inter-surface articulating joint implant to partially float in synovial fluid between said articulating surfaces of said acetabulum socket and said femoral head and to partially contact at least one of said acetabulum socket and said femoral head.

172-173. (canceled)

174. A method of implanting an inter-surface articulating joint implant, said method comprising the steps of: surgically inserting said inter-surface articulating joint implant between a tibia and a femur head when said inter-surface articulating joint implant does not necessarily conform to corresponding shapes of articulating surfaces of said tibia and said femur head; by means of at least some articulation of the knee joint, causing the shape of said inter-surface articulating joint implant to become increasingly conformal to said corresponding shapes of said articulating surfaces of said tibia and said femur head; and causing said inter-surface articulating joint implant to float in synovial fluid between said articulating surfaces of said tibia and said femur head.

175. A method of implanting an inter-surface articulating joint implant, said method comprising the steps of: surgically inserting said inter-surface articulating joint implant between a tibia and a femur when said inter-surface articulating joint implant does not necessarily conform to corresponding shapes of articulating surfaces of said tibia and said femur; by means of at least some articulation of the knee joint, causing the shape of said inter-surface articulating joint implant to become increasingly conformal to said corresponding shapes of said articulating surfaces of said tibia and said femur; and causing said inter-surface articulating joint implant to partially float in synovial fluid between said articulating surfaces of said tibia and said femur and to partially contact at least one of said tibia and said femur.

176-177. (canceled)

178. A method of implanting a meniscus implant, said method comprising the steps of: surgically inserting said meniscus implant between a tibia and a femur when said meniscus implant does not necessarily conform to corresponding shapes of articulating surfaces of said tibia and said femur; by means of at least some articulation of the knee joint, causing the shape of said meniscus implant to become increasingly conformal to said corresponding shapes of said articulating surfaces of said tibia and said femur; and causing said meniscus implant to float in synovial fluid between said articulating surfaces of said tibia and said femur.

179. A method of implanting a meniscus implant, said method comprising the steps of: surgically inserting said meniscus implant between a tibia and a femur when said meniscus implant does not necessarily conform to corresponding shapes of articulating surfaces of said tibia and said femur; by means of at least some articulation of the knee joint, causing the shape of said meniscus implant to become increasingly conformal to said corresponding shapes of said articulating surfaces of said tibia and said femur; and causing said meniscus implant to partially float in synovial fluid between said articulating surfaces of said tibia and said femur and to partially contact at least one of said tibia and said femur.

180-181. (canceled)

182. A method of employing a shock-absorbing ball implant, said method comprising the steps of: surgically inserting said shock-absorbing ball implant and rigidly mounting it onto a stem; disposing said shock-absorbing ball implant with an articulating surface thereof in articulating engagement with a socket; and by walking or running, intermittently applying a force on said shock-absorbing ball implant via said socket, thereby producing deformation of said shock-absorbing ball implant and consequent temporary reshaping of said articulating surface, said deformation producing pumping of synovial fluid into an articulation region between said articulating surface and said socket.

183. (canceled)

184. A method of employing an individually in-situ sizable ball implant, said method comprising the steps of: surgically inserting said individually in-situ sizable ball implant and rigidly mounting it onto a stem; disposing said individually in-situ sizable ball implant with an articulating surface thereof in articulating engagement with a socket; and selectably filling at least a portion of said individually in-situ sizable ball implant to cause said articulating surface to have an individually preferred configuration adapted to said socket.

185. (canceled)

186. A method of surgically reaming a bone socket comprising the steps of: providing a reamer including: a bone anchor assembly including a generally spherical bone engagement surface and a central shaft extending along a longitudinal axis, said central shaft having a first threaded portion thereon; a rotational driving assembly, mounted for rotation about said central shaft and including a first handle fixedly associated with an elongate hollow shaft, which is sized to rotationally accommodate said central shaft, said hollow shaft terminating in a rotational driving plate; a rotational and axial driving element, coupled for rotation together with said rotational driving plate, said rotational and axial driving element including a second threaded portion, which threadably engages said first threaded portion, said rotational driving plate being formed with a plurality of axially extending portions which extend into corresponding axially extending sockets formed in said rotational and axial driving element and a plurality of first slidable knife support channels and said rotational and axial driving element being formed with a plurality of correspondingly positioned second slidable knife support channels, which extend generally at an angle with respect to said first slidable knife support channels; and a plurality of knives, each slidably seated in a pair of corresponding ones of said first and second slidable knife support channels and mounted on a resilient knife support which permits simultaneous radially outward and rotational displacement of said plurality of knives in response to simultaneous axial and rotational movement of said rotational and axial driving element in threaded engagement with said first threaded portion in response to rotation of said first handle; placing said reamer with said bone anchor assembly in a bone socket to be reamed; pushing said first handle axially, thereby causing said generally spherical bone engagement surface to engage a bone surface at said bone socket, thereby anchoring said bone anchor assembly against rotation with respect to said bone socket; producing engagement of radial bone engagement tooth portions of said bone anchor assembly with side surfaces of said bone socket; and rotating said first handle about said longitudinal axis, thereby causing rotation of said rotational and axial driving element about said longitudinal axis and consequent corresponding axially forward displacement of said rotational and axial driving element due to threaded engagement of said threaded portion said central shaft with said second threaded portion, said forward displacement driving said plurality of knives which are slidably seated in said first and second slidable knife support channels in a radially outward direction into cutting engagement with said bone socket and said rotation of said first handle about said longitudinal axis causing rotation of said rotational and axial driving element about said longitudinal axis and consequent corresponding axially forward displacement thereof, driving said plurality of knives into cutting engagement with said bone socket, thus producing a circumferential channel therein.

187. A method for assembly of a socket implant assembly, said method comprising the steps of: mounting a flexible central element on an assembly fixture in a manner preventing relative rotation between said flexible central element and said assembly fixture; mounting a bottom element onto said flexible central element; mounting an inner implant element onto said flexible central element and said bottom element; mounting an outer implant element over said inner implant element and in engagement with said flexible central element; and mounting outer elements onto said flexible central element.

188. A method for mounting a socket implant assembly onto an implanter comprising the steps of: providing said socket implant assembly comprising a flexible central element, a bottom element, an inner implant element, an outer implant element and outer elements; providing said implanter comprising: a central shaft extending along an axis and having an outer facing threaded portion and an end portion having a reduced radius and defining a shoulder with respect to the remainder of said central shaft, said end portion having an end surface formed with a bevel; a rotational driving assembly mounted onto said central shaft for driving rotation thereof, said rotational driving assembly including a handle fixed to said central shaft; and a sleeve disposed about said central shaft, said sleeve including a generally uniform inner surface, an inner facing threaded portion and an outer surface which is conditioned to provide a gripping surface for user engagement during implanting, said outer surface including a peripheral protrusion of uniform radius and spaced therefrom along said axis a beveled protrusion, which tapers inwardly and towards a bottom edge of said sleeve, a space being provided between said peripheral protrusion and said beveled protrusion and defining a recess; and providing relative axial displacement of said socket implant assembly and said implanter along said axis, producing snap-fit engagement therebetween.

189. A method for implanting a socket implant, said method comprising the steps of: reaming a bone socket of a patient in order to define a circumferential groove therein; axially inserting, a relatively resilient outer implant element into said bone socket and producing snap fit engagement of at least one circumferential protrusion of said relatively resilient outer implant element with said circumferential groove; and axially producing engagement of a relatively rigid inner implant element with said outer implant element, thereby to retain said outer implant element in engagement with said bone socket.

190-192. (canceled)

193. A method of implanting an orthopedic implant comprising: attaching an implant element to at least one bone, said implant element including at least one throughgoing bone growth region adapted to be located in bone growth communication with said at least one bone; and enhancing bone growth in said bone growth region such that said bone growth therein creates a grown bone suture binding said implant element to said at least one bone.

194. A method of implanting an orthopedic implant comprising: attaching a resilient implant element to at least one bone, said resilient implant element including at least one bone growth region adapted to be located in bone growth communication with said at least one bone; and allowing bone growth in said at least one bone growth region such that said bone growth therein creates a grown bone lock binding said resilient implant element to said at least one bone.

195. A method of implanting an orthopedic implant comprising: attaching an implant element to at least one bone, said implant element including at least one bone growth region adapted to be located in bone growth communication with said at least one bone and containing grown bone enhancing material; and allowing bone growth in said at least one bone growth region such that bone growth therein creates a grown bone lock binding said implant element to said at least one bone.

196-200. (canceled)

201. A method of implanting an orthopedic implant comprising: attaching a ligament attachment assembly to at least one bone and at least one ligament, said ligament attachment assembly including at least one ligament attachment element having at least one throughgoing bone growth region adapted to be located in bone growth communication with said at least one bone; and encouraging bone growth in said at least one bone growth region thereby to create a grown bone suture binding said at least one ligament attachment element to said at least one bone.

202. A method of implanting an orthopedic implant comprising: attaching a ligament attachment assembly to at least one bone, said ligament attachment assembly including at least one ligament attachment element having at least one bone growth region adapted to be located in bone growth communication with said at least one bone; and enhancing bone growth in said at least one bone growth region, thereby creating a grown bone lock binding said at least one ligament attachment element to said at least one bone.

203. A method of implanting an orthopedic implant comprising: attaching a ligament attachment assembly to at least one bone, said ligament attachment assembly including at least one ligament attachment element having at least one bone growth region adapted to be located in bone growth communication with said at least one bone and containing grown bone enhancing material; and allowing bone growth in said at least one bone growth region to create a grown bone lock binding said at least one ligament attachment element to said at least one bone.

204-205. (canceled)

206. A method of implanting an orthopedic implant comprising: inserting a conformal, resilient element between respective articulating joint surfaces of a patient, said conformal resilient element including at least one position maintaining engagement portion; and attaching said at least one position maintaining engagement portion to patient tissue in the vicinity of said articulating joint surfaces.

207-215. (canceled)

216. A method of implanting an orthopedic implant comprising: inserting a resilient, at least partially hollow, meniscus implant element between respective articulating knee joint surfaces of a knee joint of a patient, said resilient meniscus implant element including at least one position maintaining engagement portion; and attaching said at least one position maintaining engagement portion to patient tissue in the vicinity of said articulating knee joint surfaces.

217-224. (canceled)

225. A method of implanting an orthopedic implant comprising: mounting onto a stem, a shock-absorbing ball assembly including a stem mounting portion and a multi-layer shock-absorbing, ball-defining portion defining a generally ball-shaped articulating surface.

226-231. (canceled)
